(19) United States
(12) Patent Application Publication
(10) Pub. No.: US 2003/0108521 A1
(43) Pub. Date: Jun. 12, 2003

(54) Adenovirus protein IX, its domains involved in capsid assembly, transcriptional activity and nuclear reorganization

(76) Inventor: Manuel Rosa Calatrava, Strasbourg (FR)

Correspondence Address: BURNS DOANE SWECKER & MATHIS L L P, POST OFFICE BOX 1404, ALEXANDRIA, VA, 22313-1404, US

(21) Appl. No.: 10/156,824

(22) Filed: May 30, 2002

Related U.S. Application Data
(60) Provisional application No. 60/293,974, filed on May 30, 2001.

(51) Int. Cl. A61K 48/00; A61K 39/12; C07H 21/04; C12P 21/02; C12N 5/06; C07K 14/075; C12N 7/00; C12N 15/861

(52) U.S. Cl. 424/93.2; 424/186.1; 435/69.3; 435/325; 435/320.1; 435/235.1; 435/456; 530/350; 536/23.72

(57) ABSTRACT

Described are adenovirus pIX proteins which are modified by mutating one or more amino acids and/or by the inclusion of a binding moiety. Preferably, said modification is carried out in the N-terminal part or in the C-terminal leucine-repeat of the pIX protein. It is described that viruses or virus-like particles containing such a modified pIX protein show an improved gene delivery efficiency. Furthermore, described are corresponding adenoviral vectors, viruses or virus-like particles, host cells, complementation cell lines and methods for producing such viruses or virus-like particles. In addition, described are pharmaceutical compositions comprising an adenoviral vector, virus or virus-like particle, host cell or complementation cell line as mentioned above and therapeutical applications thereof.

The invention relates to new recombinant and mutant adenovirus pIX polypeptides and nucleic acid sequences encoding them, to new vectors comprising said nucleic acid sequence, and to virus or virus-like particles, in particular adenoviral or pseudo adenoviral particles comprising said pIX polypeptide or said nucleic acid sequence. Furthermore, the invention relates to a method for preparing an adenoviral or pseudo adenoviral particle; to cells and compositions comprising said polypeptide, nucleic acid sequence, virus, particle or pseudo particle; to compositions comprising said cells as well as to therapeutic or prophylactic uses. The invention is of very special interest with regard to prospects for gene therapy or vaccination using gene transfer, in particular in human.

Gene therapy, and more widely gene vaccination, can be defined as the transfer of genetic material into a cell or an organism to treat or prevent a genetic or acquired disease, or to circumvent molecular, cellular or organ disorders. The possibility of treating human diseases or disorders by gene therapy has changed in a few years from the stage of theoretical considerations to that of clinical applications. The first protocol applied to man was initiated in the USA in September 1990. It concerned a patient who was genetically immunodeficient as a result of a mutation affecting the gene encoding adenine deaminase (ADA). This first encouraging experiment has been followed by numerous new applications and promising clinical trials based on gene therapy which are currently ongoing (see for example clinical trials listed at http://cnetdb.nci.nih.gov/trialsrch.shtml or http ://www.wiley. co.uk/genetherapy/clinical/).

Successful gene therapy principally depends on the efficient delivery of a therapeutic gene of interest to make its expression possible in cells of a living organism. Therapeutic genes can be transferred into cells using a wide variety of vectors resulting in either transient expression or permanent transformation of the host genome. During the past decade, a large number of viral, as well as non-viral, vectors has been developed for gene transfer (see for example Robbins et al., 1998, Tibtech 16, 35-40 and Rolland, 1998, Therapeutic Drug Carrier Systems 15, 143-198 for reviews).

Most of the intracellular gene delivery mechanisms used to date relate to the first category, i.e. viral vectors, especially adeno- and retroviral vectors. Viruses have developed diverse and highly sophisticated mechanisms to achieve transport across the cellular membrane, escape from lysosomal degradation, delivery of their genome to the nucleus and, consequently, have been used in many gene delivery applications. Their structure, organization and biology are described in the literature available to a person skilled in the art.

One of the most widely used vectors for in vivo gene transfer is a replication-deficient adenoviral vector. Originally, adenoviruses have been detected in many animal species. They are non-integrative and not very pathogenic. They are able to infect a variety of cell types, dividing as well as quiescent cells. They have a natural tropism for airway epithelia. In addition, they have been used as live enteric vaccines for many years with an excellent safety profile. Finally, they can be grown easily and purified in large quantities. These features have made adenoviruses particularly appropriate for use as gene therapy vectors for therapeutic and vaccine purposes. A number of adenoviruses are now well characterized genetically and biochemically. This is, for example, the case with human adenovirus type 5 (Ad5), the sequence of which is disclosed in the Genbank data bank under reference M73260 (see FIG. 6).

The adenoviral genome consists of a linear double-standed DNA molecule of approximately 36kb carrying more than about thirty genes necessary to complete the viral cycle. The early genes are divided into 4 regions dispersed in the adenoviral genome (E1 to E4) which altogether contain 6 transcription units directed by their own promoters. The E1, E2 and E4 regions are essential for viral replication whereas the E3 region, which is believed to modulate the anti-viral host immune response, is dispensable for viral growth in vitro. The late genes (L1 to L5) encode in their majority the structural proteins constituting the viral capsid. They overlap at least in part with the early transcription units and are transcribed from a unique promoter (MLP, for Major Late Promoter). In addition, the adenoviral genome carries at both extremities cis-acting regions essential for DNA replication. These are the 5′ and 3′ ITR (Inverted Terminal Repeat) and a packaging sequence following 5′ ITR.

The E1 early region is located at the 5′ end of the adenoviral genome, and contains 2 viral transcription units, E1A and E1B, respectively. This region codes for proteins which participate very early in the viral cycle and are essential to the expression of almost all the other genes of the adenovirus. In particular, the E1A transcription unit codes for a protein which transactivates the transcription of the other viral genes, in particular by inducing transcription from the promoters of the E1B, E2A, E2B and E4 regions.

The products of the E2 region, which also comprises two transcription units, E2A and E2B, are directly involved in the replication of the viral DNA. This region governs, in particular, the synthesis of a 72 kDa protein, which displays a strong affinity for single-stranded DNA, and of a DNA polymerase.

The E3 region is not essential to the replication of the virus. It codes for at least six proteins which appear to be responsible for the inhibition of the host's immune response to an adenovirus infection. In particular, the gpl9 kDa glycoprotein appears to prevent the CTL response which is responsible for the cytolysis of infected cells by the host's cytotoxic T cells.

The E4 region is believed to be involved in viral DNA replication, late MRNA synthesis, viral assembly and the shut off of host protein synthesis. It is a complex transcription unit which encodes a variety of polypeptides. Those encoded by the open reading frames (ORFs) 6 and 7 are assumed to compete with the cellular RB protein for binding to the E2F transcription factor, thereby conferring a transactivating function. The expression product of ORF4 is able to bind and regulate the cellular phosphatase 2A to modulate the activity of viral (E1A) and cellular transcription factors. The polypeptides encoded by ORFs 3 and 6 are essential to viral growth because of their capability to maturate the primary 28 kb transcript derived from the adenoviral genome and to promote its export into the cytoplasm. Their absence may be complemented in trans to allow viral growth. In addition, the ORF6 polypeptide interacts with the E1B encoded 55K polypeptide to form a complex that facilitates the cytoplasmic accumulation of late messengers at the expense of cellular MRNA.

The adenoviral vectors presently used in gene therapy protocols lack most of the E1 region in order to avoid their dissemination in the environment and the host body. Additional deletions in the E3 region allow to increase the cloning capacity. The gene of interest is introduced into the viral DNA in place of a deleted region. The feasibility of gene transfer using these vectors designated “first generation” has been demonstrated in a number of cases.

Further constructs (“second generation vectors”) have been made that retain the cis regions necessary for viral replication (ITRs and packaging sequences) and contain substantial genetic modifications with the aim to abolish residual synthesis of viral antigens. The antigens have been postulated to be responsible for the stimulation of inflammatory responses (see for example the international application W094/28152 or U.S. Pat. No. 5,670,488 which discloses adenoviral vectors having E4 sequences partially deleted with the exception of ORF3 or ORF6/7 that do not need E4 complementation). A minimal vector deficient in all adenoviral functions can also be considered. For a general revue, one can refer to Lusky et al. J. Virol 72 (1998) 2022-2032).

Finally, the infectious cycle of the adenovirus takes place in 2 steps:

the early phase which precedes initiation of replication of the adenoviral genome, and which permits production of the regulatory proteins participating in the replication and transcription of the viral DNA, and

once replication of the viral DNA has been initiated, transcription of the late genes begins.

The late genes occupy the majority of the adenoviral genome and partially overlap the transcription units of the early genes. However, they are transcribed from different promoters and according to an alternative mode of splicing, so that the same sequences are used for different purposes. Most of the late genes are transcribed from the major late promoter (MLP). This promoter permits the synthesis of a long primary transcript, which is then matured into about twenty messenger RNAs (mRNAs) from which the capsid proteins of the virion are produced. The gene coding for structural protein IX (pIX) of which the capsid is composed is located at the 5′ end of the adenoviral genome and overlaps the E1B region at its 3′ end. The protein IX transcription unit utilizes the same transcription termination signal as the E1B transcription unit.

pIX is a small polypeptide of 140 amino acid residues (14.3 kDa) that is incorporated into the viral particle or pseudo particle, or capsid. More specifically, said pIX polypeptide is associated with hexon proteins to form group-of-nine hexons (GON) that make up the central region of each facet of the icosahedron (Boulanger et al., 1979, J. General Virology, 44, 783-800 ; Burnett, 1985, J. Molecular Biology, 185, 125-143 ; Burnett et al., 1985, J. Molecular Biology, 185, 105-123). Precise determination of the stochiometry of this assembly has revealed that there are 12 molecules of pIX, organized as four trimers per GON, and therefore 240 molecules per capsid (Steward et al., 1991, Cell, 67, 145-154). The pIX polypeptide acts as a capsidic cement and thereby enhances thermal stability of the viral particles (Colby et al, 1981, J. Virol., 39, 977-980 ; Furcinitti et al., 1989, EMBO J., 8, 3563-3570). However, while it is well established that pIX is essential for building the adenoviral capsid, no result is yet available permitting to precisely delineate the “minimal” pIX sequence which is actually necessary for obtaining correct encapsidation of the adenoviral genome, or at least for the formation of particles (whether or not containing the adenoviral genome ).

Additionally, observations have strongly suggested that pIX is furthermore involved in the infectious cycle (Lutz et al., 1997, J. of Virology, 71, 5102-5109) : (i) the gene encoding pIX is the only gene encoding a structural protein which is not expressed under the control of the adenoviral major late promoter (MLP), (ii) its expression pattern follows a time course different from that of the other structural proteins and begins at intermediate times post-infection (p.i.), and actually much earlier than that of all the other structural proteins; (iii) finally, pIX accumulates in the nuclei of infected cells with a speckled distribution. In agreement with this nuclear localization, Lutz et al. (1997, Journal of Virology 71, 5102-9) have previously shown that pIX is a transcriptional activator of several viral and cellular TATA-containing promoters, among which are the genes controlled by the E1a, E4 and MLP promoters.

The design of viral gene therapy vectors which are capable to deliver therapeutic genes to a specific cell represents one of the main interest and challenge in today's gene therapy research. The use of targeting vectors would limit the vector spread, thus increasing therapeutic efficacy for the desired target cells and minimizing potential side effects.

The broad tissue tropism of adenoviruses may turn disadvantageous when genes encoding potentially harmful proteins (e.g. cytokines, cytotoxic proteins, suicide gene products) are expressed in surrounding normal tissues. Moreover, the overall in vivo efficiency of gene delivery might be reduced by a significant dilution of the virus in the organism due to the transduction of non-target cells. The development of adenovirus vectors with targeted infectivity capacities would therefore greatly improve the safety and efficacy of some current gene therapy strategies. Thus, targeting adenoviral vectors may improve gene therapy procedures by providing augmented infectivity of poorly transduced cells (e.g. tumor cells) and decreased toxicity to normal tissues.

The specificity of infection of the adenoviruses is determined by the attachment of the virions to cellular receptors present at the surface of permissive cells. In this regard, the fiber present at the surface of the viral capsid plays a critical role in cellular attachment (Defer et al. J. Virol. 64 (1990) 3661-3673) and penton-base promotes internalization through the binding to the cellular integrins (Mathias et al. J. Virol. 68 (1994) 6811-6814). Recent studies have presumed the use of the coxsackie virus receptor (CAR) by type 2 and 5 adenoviruses (Bergelson et al; Science 275 (1997) 1320-1323). In particular, the initial attachment of the adenovirus particle to the cell surface is mediated by the binding of the knob region of the viral fiber protein to the ubiquitous CAR (Bergelson et al., 1997, Science 275, 1320-1323; Tomko et al., 1997, Proc. Natl. Acad. Sci. USA 94, 3352-3356) CAR was identified as the primary receptor for adenovirus serotype C fibers (e.g. Ad5) and the cell-surface heparan sulfate glycosaminoglycans (HSG) which was recently shown to interact with AdS fiber and facilitate virus binding to cells (Dechecchi et al., 2000, Virology 268, 382-390 ; Dechecchi et al., 2001, J. Virol. 75, 8772-8780). However, other surface proteins may also be involved in fiber attachment, for example, the alpha2 domain of the class I histocompatibility antigens as identified by Hong et al. (EMBO J. 16 (1997) 2294-2306). The fiber is composed of 3 regions (Chroboczek et al. Current Top. Microbiol. Immunol. 199 (1995) 165-200): the tail at the N-terminus of the protein which interacts with penton base and ensures the anchorage in the capsid, the shaft composed of a number of beta-sheet repeats and the knob which contains the trimerization signals (Hong et al. J. Virol. 70 (1996) 7071-7078) and the receptor binding moiety (Henri et al. J. Virol. 68 (1994) 5239-5246 ; Louis et al. J. Virol. 68 (1994) 4104-4106).

The almost ubiquitous distribution of the CAR cellular receptor is thought to be primarily responsible for the broad cell tropism of the human serotype C adenoviruses. Consistent with this notion, the absence or reduced expression of this receptor has been shown to correlate with the poor sensitivity of certain cell types (e.g. lymphocytes, smooth muscle cells) to adenovirus transduction (Leon et al., 1998, Proc. Natl. Acad. Sci. USA 95, 13159-13164 ; March et al., 1995, Hum. Gene Ther. 6, 41-63). Moreover, numerous studies have now reported that primary tumor cells express only low levels of CAR (Li et al., 1999, Cancer Res. 59, 325-330 ; Miller et al., 1998, Cancer Res, 58, 5738-5748).

The inability of adenovirus to efficiently transduce specific cell populations, together with its lack of strict tissue specificity, have thus stimulated increasing efforts to redirect the adenovirus tropism from its natural receptors to specific cell surface molecules.

Recently, specific fiber mutations which eliminate the interaction with CAR were identified, demonstrating that the CAR binding site of the fiber knob domain can be mutated without adversely affecting the quaternary structure and overall conformation of the purified recombinant protein (WO98/44121, WO01/16344 and WO01/38361). For example, fiber proteins carrying amino acid substitutions in the AB loop (involving Ser408 and Pro4O9), in the DG loop (e.g. involving Tyr 477) and in beta-strand F (e.g. involving Leu 485) or having two consecutive amino acids deleted in the DG loop were shown to alter CAR binding (Bewley et al., 1999, Science 286, 1579-1583; Kirby et al., 1999, J. Virol 73, 9508-9514 Kirby et al., 2000, J. Virol. 74, 2804-2813). These viruses are structurally identical to native viruses and therefore constitute appropriate substrates for the insertion of targeting ligands (binding moieties) in the mutated fibers. In this respect, several groups have reported that insertion of stretches of lysine residues at the C-terminal end of the knob could lead to the generation of high titer viruses that were characterized by a 10 to 300 fold increase in their efficiency of infection of CAR-deficient cells, such as macrophages, endothelial cells, smooth muscle cells or T lymphocytes (Wickham et al., 1997, J. Virol 71, 8221-8229; Yoshida et al., 1998, Hum. Gene Ther. 9, 2503-2515; Wickham et al., 1996, Nature Biotechnology 14, 1570-1573; Bouri et al., 1999, Hum. Gene Ther.10, 1633-1640). Furthermore, the introduction of a peptide ligand binding the transferrin receptor in the fiber HI loop was shown to facilitate gene transfer to cells which over-express this receptor (Xia et al., 2000, J. Virol. 74, 11359-11366). Similarly, a HUVEC cell-binding peptide allowed a significant increase of the transduction efficiency of the retargeted vector towards these cells which are normally refractory to transduction (Nicklin et al., 2000, Circulation 102, 231-237).

Further scientific teams addressed the adenoviral targetting issue by modifying the adenoviral fiber protein. For example, U.S. Pat. No. 5,885,808 describes an adenovirus, or adenovirus-like particle, having a penton fibre comprising a modified binding specificity conferred by a binding moiety which is heterologous to the adenovirus and is incorporated as a fusion protein with the fibre protein allowing the adenovirus or adenovirus-like particle to bind to a target cell which is not the natural host cell of the virus, characterized in that the said penton fibre is modified by the insertion or deletion or substitution of amino acid residues that disrupt the host-cell binding function so that the adenovirus or adenovirus-like particle is substantially incapable of binding the natural host cell.

Similarly, U.S. Pat. No. 6,057,155 provides a chimeric adenovirus fiber protein, which differs from the wild-type coat protein by the introduction of a non-native amino acid sequence in a conformationally-restrained manner. A vector comprising such a chimeric fiber protein is able to direct entry into cells more efficiently than a corresponding vector that is identical except for comprising a wild-type adenovirus fiber protein. The non-native amino acid sequences may represent a peptide motif that comprises an epitope for an antibody or a ligand for a cell surface receptor, that can be employed in cell targeting.

U.S. Pat. No. 5,756,086 discloses an adenovirus, wherein the adenovirus fiber protein includes a ligand which is specific for a receptor located on the surface of a desired cell type. The adenovirus may have at least a portion of the adenovirus fiber protein removed and replaced with a ligand which is specific for a receptor of a desired cell type, or the adenovirus may include a fusion protein of the adenovirus fiber protein and the ligand. Such an adenovirus may also include a gene(s) encoding a therapeutic agent(s) and may be “targeted” in order to deliver such gene(s) to a desired cell type.

However, it is to be noted that the prior art does not teach to construct targeted adenoviral vectors comprising a targeting ligand in the pIX protein.

Nevertheless, there is still an ongoing need for further optimized vectors based on adenoviruses in order to improve gene therapeutic applications.

Thus, the technical problem underlying the present invention is the provision of alternative means and methods for producing adenovirus-based viruses and virus-like particles suitable for efficient gene delivery. Preferably, viruses or virus-like particles show an improved gene delivery efficiency as compared to prior art viruses or virus-like particles, in particular with regard to targeting capacity and/or reduction of undesired side effects.

The solution to this technical problem is achieved by providing the embodiments characterized in the claims.

Accordingly, the present invention relates to adenovirus pIX proteins which are modified by mutation of one or more amino acids of said pIX protein as compared to the corresponding wild-type pIX protein and/or so as to comprise a binding moiety, wherein the presence of said modified pIX protein in a virus or virus-like particle results in an improved gene delivery efficiency of said virus or virus-like particle in a target cell as compared to the gene delivery efficiency of a corresponding virus or virus-like particle containing said corresponding wild-type pIX protein.

The invention is based on the experimental data presented in the appended Examples. The experiments were conducted in order to precisely delineate the functional domains of piX that are responsible for the structural and transcriptional properties of the protein, in particular by performing an extensive mutational analysis of the pIX coding sequence. More precisely, it has been shown that the highly conserved N-terminal part of the protein is essential for the capsidic structural properties, whereas the C-terminal leucine-repeat (putative coiled-coil domain) is critical for the transactivating function and can be modified without altering the structural function of pIX.

It is one of the objects in the design of adenoviral gene therapy vectors to delete unnecessary parts of the viral genome. In the experiments underlying the present invention it has been shown that parts of the pIX protein can be deleted or otherwise modified without impairing for example the viability or stability of the end-product, e.g. an adenoviral particle. Therefore, the present invention proposes to modify the adenoviral pIX protein by inserting a ligand moiety in order to modify adenovirus specificity (e.g. enhanced transduction of poorly infected cells or restriction of infection to specific cells or categories of cells). In order to preserve the functionality of the pIX protein during the adenovirus life cycle, specific locations in the pIX protein have been identified for incorporation of the targeting binding moiety, especially within or at the C-terminal part of the pIX protein. Moreover, the present invention also provides specific mutations of the pIX protein, especially in the C-terminal leucine-repeat domain, which may enhance the presentation of such a binding moiety at the surface of the virus or virus-like particle.

In the context of the present invention, the term “adenovirus pIX protein” refers to a pIX protein encoded by an adenoviral genome which is known to be integrated into the capsid of virus or virus-like particles. The present invention encompasses the full length adenoviral pIX which is encoded by the complete coding sequence (i.e. from the initiator ATG codon to the stop codon). However, it is possible to employ a fragment thereof generated by internal deletion, or truncation having the properties as described herein. For illustrative purpose, the pIX-encoding sequence can be isolated from an adenoviral genome by conventional recombinant techniques. The pIX gene is present at the left end of the adenoviral genome positioned between E1B and E2 regions, e.g. from nucleotides (nt) 3609 to 4031 in the Ad5 genome (see FIG. 6).

The modified adenoviral pIX protein of the invention may originate (i.e. the source sequence for constructing the pIX protein of the invention may be obtained) from an adenovirus of human or animal (e.g. canine, avian, bovine, murine, ovine, porcine, simien and the like) or may be a hybrid comprising fragments of diverse origins. For instance, the adenovirus can be of subgroup A (e.g. serotypes 12, 18, 31), subgroup B (e.g. serotypes 3, 7, 11, 14, 16, 21, 34, 35), subgroup C (e.g. serotypes 1, 2, 5, 6), subgroup D (e.g. serotypes 8, 9, 10, 13, 15, 17, 19, 20, 22-30, 32, 33, 36-39, 42-47), subgroup E (serotype 4), subgroup F (serotype 40, 41), or any other adenoviral serotype. Preferably, however, the modified pIX of the invention originates from an adenovirus of subgroup C, with a special preference for Ad2 or Ad5 serotype.

Although the pIX protein may vary between the different human and animal adenovirus strains, it can be identified on the basis of nucleotide and amino acid sequences available from different sources (e.g. databases such as GenBank and literature publications) or by homology with the well characterized Ad5 sequences (disclosed in Genbank under accession number M73260 or in Chroboczek et al., 1992, Virology 186, 280-285). By way of illustration, the pIX protein of Ad5 includes 140 amino acid residues including the initiator Met residue (as shown in SEQ ID NO: 1). Moreover, FIG. 1 indicates the amino acid sequences of pIX proteins of a number of human and animal adenovirus strains.

The term “modified by mutation of one or more amino acids” refers to one or more deletions, substitutions or insertions of one or more residues as compared to the wild type pIX protein, or any combination of these possibilities. When several mutations are contemplated, they can concern consecutive residues and/or non consecutive residues at any location of the pIX sequence. Mutation may be made in a number of ways known to those skilled in the art using recombinant techniques, including for instance by enzymatically cleaving from the pIX-encoding nucleotide sequence followed by modification and ligation of the fragment obtained, by site-directed mutagenesis (e.g. the “QuickChange site-directed mutagenesis” system of Stratagene) or by PCR techniques. Deletion mutations can comprise from about 1 to 30 amino acid residues, preferably not exceeding 15 amino acids.

According to one embodiment, the modification results in the insertion of a binding moiety into the pIX sequence, within or to the N-terminal part or within or to the C-terminal part of the pIX protein (as defined hereinafter), with a special preference for the latter. In this respect, the binding moiety can be inserted at the C-terminus or within about the 30 and more preferably, about the 20 residues preceding the C-terminus. The insertion of the binding moiety can be made between two pIX residues or by replacing one or more pIX residues.

According to another embodiment, the modification results in the substitution of at least one amino acid residue as compared to the wild type pIX protein. Preferably, such a mutation is located within the C-terminal leucine repeat of the pIX protein.

In another specific embodiment, the modification results in the mutation of the stop codon of the pIX sequence, in order to suppress its stop activity (e.g. by mutating the stop codon in a amino acid encoding codon). In this embodiment, translation will continue beyond the native stop codon and the the polyA sequence naturally present downstream of the pIX coding sequence will be translated into a stretch of polylysine which could be use as a binding moiety connected to the C-terminus of the pIX protein.

Within the meaning of the invention, the term “improved gene delivery efficiency” refers to the property of a virus or virus-like particle harbouring a modified pIX protein of the invention to infect a target cell and/or to deliver a gene of interest to a target cell (1.) more specifically (i.e. showing an increased ratio of infection and/or gene delivery between target and non-target cell) and/or (2.) more efficiently (i.e. infection and/or gene delivery is enhanced in absolute terms) as compared to a corresponding virus-like particle that does not harbour the modified pIX protein. The improved gene delivery efficiency can be easily determined by comparing, using the techniques of the art, the infection property or the propensy to deliver a given gene of interest (e.g. a reporter gene) of the virus or virus-like particle harboring the modified pIX as compared to a related virus or virus-like particle harbouring a non modified (wild-type) pIX protein to target cells and non target cells, either in vitro (e.g. in cultured cells) or in vivo (e.g. in animal models) and under the same experimental conditions. Suitable techniques include cell infectivity studies with appropriate cell lines, evaluation of cell attachment for example using labeled viruses (e.g. labeled with ³H thymidine, as described in Roelvin et al., 1996, J. Virol. 70, 7614-7621). For instance when the pIX protein is modified so as to comprise a binding moiety, such assays involve exposition of the viruses to a target cell (e.g. displaying at its surface the anti-ligand molecule recognized by the binding moiety) under standard conditions of infection. As a result, a virus or virus-like particle harboring a modified pIx protein of the present invention shows a propensity to infect the target cell with a better efficiency than a virus or virus-like particle harboring a non modified (wild-type) pIX protein, which means that the virus or virus-like particle harboring a modified pIX protein infects more efficiently or more rapidely said target cells than non target cells (that do not display at their surface such an anti-ligand molecule), whereas a virus or virus-like particle harboring a wild-type pIX protein will infect said target cells with a lower or a similar efficiency compared to non-target cells. Alternatively, the improved gene delivery efficiency provided by the modified pIX protein of the invention can also be evaluated by measuring the level of gene transfer (e.g. using a reporter gene). Such a measurement can be done using any techniques in the art including Western blotting, ELISA, immunodetection, enzymatic detection, biological activities and the like.

Advantageously, the modified pIX protein of the present invention improves gene delivery efficiency when the infection or delivery of a gene of interest to a target cell measured with a virus or virus-like particle harbouring such a modified pIX is substantially increased by at least a factor of two as compared to that observed with a virus or virus-like particle harbouring a wild-type adenoviral pIX. Preferably, it is increase by at least about five, more preferably by at least about one order of magnitude, even more preferably by at least about two orders of magnitude as compared to that observed with the corresponding wild-type virus or virus-like particle.

A “target cell” as used herein is a cell where infection of a virus or virus-like particle harboring the modified pIX protein of the invention is expected. <<Target cell>> refers to a single entity, or can be part of a larger collection of cells. Such a larger collection of cells can comprise, for instance, a cell culture (either mixed or pure), a tissue (e.g., epithelial or other tissue), an organ (e.g., heart, lung, liver, urinary bladder, muscle or other organ), an organ system (e.g., circulatory system, respiratory system, gastrointestinal system, urinary system, nervous system, integumentary system or other organ system), or an organism (e.g., a mammal, particularly a human, or the like). In the context of the present invention, the target cell is preferably a tumoral cell. When the pIX protein is modified so as to comprise a binding moiety, the “target cell” may designate a unique type of cell or a group of different types of cells having as a common feature on their surface anti-ligand molecule(s) recognized by the binding moiety(s) comprised in said pIX protein.

The term “and/or” whereever used herein includes the meaning of “and”, “or” and “all or any other combination of the elements connected by said term”.

The term “about” or “approximately” as used herein means within 20%, preferably within 10%, and more preferably within 5% of a given value or range.

The results disclosed in the present application on pIX functions and mapping of said functions on the adenoviral genome allow it to produce new virus and virus-like particles at least some of which can bind the target cell with high specificity and may deliver genetic material to the target cell; at least some of the viruses and virus-like particles may bind and deliver genetic material to the target cell, preferably without substantially binding to the natural host cell of the virus.

The term “binding moiety” means a molecule that is exposed on the surface of the virus or virus-like particle which is able to bind to a molecule on the target cell. The “binding moiety” may be a molecule on the virus or virus-like particle that is modified in such a way that its binding specificity is changed, or it may be a molecule added to, and exposed on the surface of, the virus or virus-like particle to provide a new binding specificity.

It is further preferred that the binding moiety is joined or fused to the virus or virus-like particles directly or indirectly by a spacer group.

In the context of the present invention, the term “adenovirus pIX protein comprising a binding moiety” means that the modified pIX protein of the invention is covalently bound to a binding moiety. Preferably, the covalent bond to the binding moiety is located within the N-terminal part or the C-terminal part, preferably the C-terminal leucine-repeat, of the pIX protein as defined further-below.

In a preferred embodiment, the binding moiety is fused to the amino acid sequence of the protein, preferably within or to the N-terminal part or the C-terminal part, preferably the C-terminal leucine-repeat, of the pIX protein. In other words, this preferred embodiment means that the mutation of one or more amino acids of the pIX protein as mentioned above results in the presence of a binding moiety in the pIX protein.

It is particularly preferred that the binding moiety comprised by the pIX protein of the invention is capable to bind a target cell as it is described in detail further below.

Any cell-binding protein or peptide or carbohydrate such as an oligosaccharide or lipid may be useful as a binding moiety, preferably for targeting the virus or virus-like particle to the cell, whereby polypeptides are preferred. For example, short linear stretches of amino acids, such as those constituting a peptide hormone may be useful, as may be domains of polypeptides that can fold independently into a structure that can bind to the target cell.

In one preferred embodiment, the binding moiety may be a monoclonal antibody or binding fragment thereof, an ScFv (single chain Fv fragment), a dAb (single domain antibody) or a minimal recognition unit of an antibody. The binding site on the target cell may be a target cell-specific antigen.

The binding moiety may be a monoclonal antibody. Monoclonal antibodies which will bind to many of these antigens are already known but in any case, with today's techniques in relation to monoclonal antibody technology, antibodies can be prepared to most antigens. The binding moiety may be a part of an antibody (for example a Fab fragment) or a synthetic antibody fragment (for example, ScFv). Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: Techniques and Applications”, J. G. R. Hurrell (CRC Press, 1982). Suitably prepared non-human antibodies can be “humanized” in known ways, for example by inserting the CDR regions of mouse antibodies into the framework of human antibodies. The variable heavy (V.sub.H) and variable light (V.sub.L) domains of the antibody are involved in antigen recognition, a fact first recognised by early protease digestion experiments. Further confirmation was found by “humanization” of rodent antibodies.

That antigenic specificity is conferred by variable domains and is independent of the constant domains is known from experiments involving the bacterial expression of antibody fragments, all of which containing one or more variable domains. These molecules include Fab-like molecules (Better et al (1988) Science 240, 1041); Fv molecules (Skerra et al (1988) Science 240, 1038); ScFv molecules where the V_{H} and V_{L} partner domains are linked via a flexible oligopeptide (Bird et al (1988) Science 242, 423; Huston et al (1988) Proc. Natl. Acad. Sci. USA 85, 5879) and dAbs comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The term “ScFv molecules” refers to molecules wherein the VH and VL partner domains are linked via a flexible oligopeptide.

It may be advantageous to use antibody fragments, rather than whole antibodies. Effector functions of whole antibodies, such as complement binding, may be removed in such fragments. ScFv and dAb antibody fragments can be expressed as fusions with other polypeptides.

Minimal recognition units may be derived from the sequence of one or more of the complementary-determining regions (CDR) of a Fv fragment. Whole antibodies, and F(ab′)₂ fragments are “bivalent”. By “bivalent”, it is meant that said antibodies and F(ab′)₂ fragments have two antigen combining sites. In contrast, Fab, Fv, ScFv, dAb fragments and minimal recognition units are monovalent, having only one antigen combining sites.

In a further preferred embodiment, the binding moiety is at least part of a ligand of a target cell-specific cell-surface receptor.

A particular cell-surface receptor can be present on a narrow class of cell types or a broader group encompassing several cell types. The present invention also encompasses the use of a binding moiety targeting cells within any organ or system, including for example, respiratory system (trachea, upper airways, lower airways, alveoli), nervous system and sensitory organs (e.g. skin, ear, nasal, tongue, eye), digestive system (e.g. oral epithelium, salivary glands, stomach, small intestines, duodenum, colon, gall bladder, pancreas, rectum), muscular system (e.g. cardiac muscle, skeletal muscle, smooth muscle, connective tissue, tendons, etc), immune system (e.g. bone marrow, stem cells, spleen, thymus, lymphatic system, etc), circulatory system (e.g. muscles connective tissue, endothelia of the arteries, veins, capillaries, etc), reproductive sytem (e.g. testis, prostate, cervix, ovaries), urinary system (e.g. bladder, kidney, urethra), endocrine or exocrine glands (e.g. breast, adrenal glands, pituitary glands), etc.

For example, binding moieties suitable for targeting liver cells include but are not limited to those derived from ApoB (apolipoprotein) able to bind to the LDL receptor, alpha-2-macroglobulin able to bind to the LPR receptor, alpha-i acid glycoprotein able to bind to the asialoglycoprotein receptor and transferrin able to bind to the transferrin receptor. A binding moiety for targeting activated endothelial cells may be derived from the sialyl-Lewis-X antigen (able to bind to ELAM-1), from VLA-4 (able to bind to the VCAM-1 receptor) or from LFA-1 (able to bind to the ICAM-1 receptor). A binding moiety derived from CD34 is useful to target the hematopoïetic progenitor cells through binding to the CD34 receptor. A binding moiety derived from ICAM-1 is more intended to target lymphocytes through binding to the LFA-1 receptor. The targeting of T-helper cells may use a binding moiety derived from HIV gp-120 or a class II MHC antigen capable of binding to the CD4 receptor. The targeting of neuronal, glial, or endothelial cells can be performed through the use of binding moieties directed for example to tissue-factor receptor (e.g. FLT-1, CD31, CD36, Cd34, CD105, CD13, ICAM-1 ; McCormick et al., 1998, J. Biol. Chem. 273, 26323-26329), thrombomodulin receptor (Lupus et al., 1998, Suppl. 2 S120), VEGFR-3 (Lymboussaki et al., 1998, Am. J. Pathol. 153, 395-403), VCAM-1 (Schwarzacher et al., 1996, Artherosclerosis 122, 59-67) or other receptors. The targeting of blood clots can be made via fibrinogen or aIIbb3 peptide. Finally, inflamed tissues can be targeted through selectins, VCAM-1, ICAM-1, etc.

Moreover, suitable binding moieties also include linear stretches of amino acids, such as polylysine, polyarginine and the like recognized by integrins. Also, a binding moiety can comprise a commonly employed tag peptide (e.g. short amino acids sequences known to be recognized by available antisera), such as sequences from glutathione-S-transferase (GST) from Shistosoma manosi, thioredoxin beta galactosidase, or maltose binding protein (MPB) from E. coli, human alkaline phosphatase, the FLAG octapeptide or hemagluttinin (HA).

It will be appreciated by those skilled in the art that binding moieties which are polypeptides may be conveniently made using recombinant DNA techniques. The binding moiety may be fused to the pIX protein of the virus or virus-like particle or they may be synthesised independently of the virus or virus-like particle, by expression from a suitable vector in a suitable host and then joined to the virus or virus-like particle.

Nucleic acid sequences encoding many of potentially useful targeting binding moieties are known, for example those for peptide hormones, growth factors, cytokines and the like and may be readily found by reference to publicly accessible nucleotide sequence databases such as EMBL and GenBank. Once the nucleotide sequence is known it is obvious to the person skilled in the art how to make DNA encoding the chosen binding moiety using, for example, chemical DNA synthetic techniques or by using the polymerase chain reaction to amplify the required DNA from genomic DNA or from tissue-specific cDNA.

Many cDNAs encoding peptide hormones, growth factors, cytokines and the like, all of which may be useful as binding moieties, are generally available from, for example British Biotechnology Ltd, Oxford, UK.

It is preferred that, when a virus or virus-like particle comprising the pIX protein of the invention binds to its target cell, it delivers its nucleic acid to said target cell, that is the target cell is infected by the virus or virus-like particle. Target cells, especially cancer cells, that are infected in this manner by the virus or virus-like particle may express viral molecules on their surface and may be recognised by the immune system and destroyed. Of course, other cytotoxic functions of the virus may also kill the cell.

Targeting can be achieved by first identifying a suitable address at the cellular surface and then constructing a virus or virus-like particle which comprises a pIX protein comprising a binding moiety that they can recognize this address. For the identification of suitable target cell-specific addresses and molecules capable of binding these addresses suitable methods are described in the art.

It has for instance been shown that a cell type or a disease-affected cell expresses unique cell surface markers. For example, endothelial cells in rapidely growing tumors express cell surface proteins not present in quiescent endothelium, .e.g. αv integrins (Brooks et al. Science 264 (1994) 569) and receptors for certain angiogenic growth factors (Hanahan Science 277 (1997) 48). Phage display library selection methods can be employed to select peptide sequences that interact with these particular cell surface markers (see for example U.S. Pat. Nos. 5,622,699 5,223,409 and 5,403,484). In this system, a random peptide is expressed on the phage surface by fusion of the corresponding coding sequence to a gene encoding one of the phage surface proteins. The desired phages are selected on the basis of their binding to the target such as isolated organ fragments (ex vivo procedure) or cultured cells (in vitro procedure). Identification of targeting peptides can also be done by an in vivo procedure that involves injecting phage libraries into mice and subsequently rescuing the bound phages from the targeted organs. Selected peptides are identified by sequencing the region of the phage genome encoding the displayed peptide. In vivo organ screening was successfully applied to isolate peptide sequences that conferred selective phage homing to the brain and kidney (Pasqualini et al., Nature 380 (1996) 364-366), to the vasculature of lung, skin and pancreas (Rajotte et al., J. Clin. Invest. 102 (1998) 430-437) and to several tumor types (Pasqualini et al., Nature Biotechnology 15 (1997) 542-546).

Furthermore, tumors could be targeted not only via their vasculature but also via the extracellular matrix (ECM) or the tumor cells themselves. Since blood vessels are constantly modified in tumors, the endothelium is locally disrupted allowing gene therapy vectors to extravasate and interact with the ECM and tumor cells. Peptides which interact with the ECM or tumor-associated cell surface markers could also be selected using the phage display technique (Christiano et al. Cancer Gene Therapy 3 (1996) 4-10 ; Croce et al. Anticancer Res. 17 (1997) 4287-4292; Gottschalk et al. Gene Ther. 1 (1994) 185-191; Park et al. Adv Pharmacol. 40 (1997) 399-435). As an example, a HWGF motif was identified as a ligand of the matrix metalloproteinases involved in tumor growth, angiogenesis and metastasis. Administration of a HWGF-comprising peptide to a tumour-bearing animal model prevents tumor growth and invasion and prolongs animal survival (Koivunen et al. Nature Biotechnology 17 (1999) 768-774).

Recently, Romanczuk et al. (Human Gene Therapy 10 (1999) 2615-2626) reported the isolation of peptides targeting the differentiated, cilliated airway epithelial cells. Coupling of the best binding peptide to the surface of a recombinant adenovirus with bifunctional polyethylene glycol (PEG) resulted in a vector able to transduce the target cells via an alternative pathway dependent on the incorporated peptide.

In cases where the binding moiety is not part of a fusion protein with the pIX protein, the binding moiety and the pIX protein may be linked together by any of the conventional ways of cross-linking polypeptides, such as those generally described in O'Sullivan et al (Anal. Biochem. (1979) 100, 100-108). For example, the binding moiety may be enriched with thiol groups and the molecule on the surface of the virus or virus-like particle, i.e. the pIX protein, may be reacted with a bifunctional agent capable of reacting with those thiol groups, for example with the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

Other chemical procedures may be useful in joining oligosaccharide and lipids to polypeptides.

Covalent coupling between the binding moiety and the pIX protein may also be performed using a polymer such as polyethylene glycol (PEG) or its derivatives (see for example WO99/40214; Bioconjugate Techniques, 1996, 606-618 ; ed G Hermanson Academic Press and Frisch et al., 1996, Bioconjugate Chem. 7, 180-186). The binding moiety and the pIX protein may also be non-covalently coupled, for example via electrostatic interactions or through the use of affinity components such as protein A, biotin/avidin, which are able to associate both partners. Immunological coupling can also be used in the context of the present invention, for example using antibodies to conjugate the selected binding moiety to the pIX protein. For example, it is possible to use biotinylated antibodies directed to a surface-exposed pIX epitope and streptavidin-labelled antibodies directed against the selected binding moiety according to the technique disclosed by Roux et al. (1989, Proc. Natl. Acad Sci USA 86, 9079). Bifunctional antibodies directed against each of the coupling partners are also suitable for this purpose.

It is preferred that the binding moiety is a polypeptide. Preferably, the binding moiety is joined to the pIX protein in that both polypeptides are produced as a fusion by techniques of genetic engineering. The use of genetic engineering allows for the precise control over the fusion of such polypeptides. Thus, in a preferred embodiment, the modified pIX protein of the invention is a fusion between a binding moiety and the unmodified pIX protein, advantageously, the binding moiety is fused to the N-terminus of the pIX protein. Even more preferably, the binding moiety is fused within or to the C-terminal part of the pIX protein. Generally, the fusion site is selected in such a way to lead to maximal presentation of the binding moiety to its corresponding cell-surface partner, and/or to not disturb the interaction with the other capsid viral proteins that are known to interact with pIX (e.g. fiber, penton base and/or hexon).

More precisely, the binding moiety can be fused to the C-terminus or between two residues located within the C-terminal part of the pIX protein, or still in replacement of one or more residues located within the C-terminal part of the pIX protein. The first embodiment can be illustrated by the fusion of the binding moiety sequence just upstream of the stop codon. Another alternative could be the mutation of the stop codon in an amino acid encoding codon, so that the pIX polyA sequence that follows the stop codon will be translated in a stretch of several lysines (a binding moiety able to recognize cell-surface integrins). The second embodiment includes insertion of a binding moiety-encoding sequence between two codons of the pIX protein, eventually through insertion of a restriction site (preferably a 6 nt length restriction site) in the pIX sequence so that the restriction site encodes one or more codons located between two residues of the wild type pIX. Such a restriction site can be used conveniently to fuse the binding moiety-encoding sequence. A convenient illustration of such a second embodiment can be provided by the insertion of a BamHI site between the codons encoding the leucine residue in position 131 and the lysine residue in position 132 of the pIX protein. An illustration of the third embodiment is the insertion of the binding moiety in replacement of the residues 128 to 140 of the wild-type pIX protein. This results in a truncated pIX protein connected at residue 127 with the binding moiety.

Preferably, the fusion is made in frame and does not disrupt the pIX open reading frame. Eventually, the binding moiety and the pIX protein can be connected through the use of one or more spacers, e.g. a first spacer at the N-terminus of the binding moiety and optionally a second spacer at the C-terminus of the binding moiety. The spacer is preferably made up of amino acid residues with high degrees of freedom of rotation, which permit the binding moiety to adopt a conformation that is recognized by its corresponding cell-surface partner. Preferred amino acid residues for the spacer are alanine, glycine, proline and/or serine. In a specific embodiment, the spacer is a peptide comprising the sequence Ser-Ala, Gly-Ser, Pro-Ser-Ala or Pro-Gly-Ser or a repetition thereof. By way of illustration, the sequence Gly-Ser-(Ser-Ala)4-Ser is suitable for such a use.

Accordingly, the invention relates to a nucleotide sequence encoding this fusion of the binding moiety and the pIX protein of the virus or virus-like particle.

The nucleotide sequence encoding the fusion of the binding moiety and the pIX protein of the virus or virus-like particle is preferably made by an alteration of the viral genome.

The nucleotide sequence may be synthesised de novo using solid phase phosphoramidite chemistry, but it is more usual for the nucleotide sequence to be constructed from two parts, the first encoding the binding moiety and the second the pIX protein of the virus or virus-like particle. The two parts may be derived from their respective genes by restriction endonuclease digestion or by other methods known by those skilled in the art such as by polymerase chain reaction.

A variety of methods have been developed to operatively link two nucleotide sequences via complementary cohesive termini. For instance, synthetic linkers containing one or more restriction sites provide a method of joining the two DNA segment together. Each DNA segment, generated by endonuclease restriction digestion, may be treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments arc then cleaved with the appropriate restriction enzyme and lifted to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA. A desirable way to generate the DNA encoding the fusion polypeptide of the invention is to use the polymerase chain reaction as disclosed by Saiki et al (1988) Science 239, 487-491.

In this method each of the DNA molecules encoding the two polypeptides to be fused are enzymatically amplified using two specific oligonucleotide primers which themselves become incorporated into the amplified DNA. The said specific primers may contain restriction endonuclease recognition sites which may then be used to join the said two DNA molecules using T4 DNA ligase as disclosed.

In a preferred embodiment, the invention concerns an adenovirus pIX protein modified by mutation of one or more amino acids of said pIX protein as compared to the wild-type pIX protein, wherein said amino acids are selected in the N-terminal part of the protein or in the C-terminal part, preferably the C-terminal leucine-repeat of the protein. Preferably, the adenovirus pIX protein of the invention comprises a binding moiety at the C-terminal part of the protein, preferably at the C-terminal leucine -repeat. In an advantageous embodiment, said binding moiety is able to bind to a target cell.

The term “N-terminal part” as used herein refers to the portion of the pIX protein extending from about amino acid residue in position 1 to about amino acid residue in position 39. With respect to Ad5 pIX protein, it refers more precisely to the portion of the pIX protein extending from about the methionine residue in position 1 to about the valine residue in position 39, as shown in SEQ ID NO: 1.

The term “C-terminal part” as used herein refers to the portion of the pIX protein extending from about the first amino acid residue of the leucine repeat domain to about the last amino acid residue of the pIX protein. With respect to Ad5 pIX protein, it refers more precisely to the portion of the pIX protein extending from about the leucine residue in position 100 to about the valine residue in position 140, as shown in SEQ ID NO: 1.

In another preferred embodiment, the invention concerns an adenovirus pIX protein modified by mutation of one or more amino acids of said pIX protein as compared to the wild-type pIX protein, wherein said amino acids are selected in the C-terminal leucine-repeat of the protein. Preferably, said modified pIX protein does not have a rigid helix structure.

The term “C-terminal leucine-repeat” as used herein refers to the portion of the pIX protein rich in hydrophobic amino acid residues (e.g. leucine and/or valine). Preferably, the C-terminal leucine-repeat contains at least the sequence “(LXXLXXXLXX)n” where X is any amino acid residue and n is between 1 to 4. With respect to Ad5 pIX protein, it refers more precisely to the portion of the pIX protein extending from about the leucine residue in position 100 to about the leucine residue in position 121, as shown in SEQ ID NO: 1.

Preferably, the mutation within the C-terminal leucine-repeat is aimed to destabilize the helix structure provided by said C-terminal leucine-repeat in the wild-type pIX protein. Such a mutation can affect one or more residue(s) involved in the helix structure, with a special preference for the hydrophobic residues such as leucine and/or valine. More precisely, such a mutation can be a deletion of all or part of the Leucine repeat domain or a mutation affecting one or more residues selected from the group consisting of the leucine in position 100, the leucine in position 103, the leucine in position 104, the glutamine in position 106, the leucine in position 107, the leucine in position 110, the glutamic acid in position 113, the leucine in position 114, the valine in position 117, the leucine in position 121, of the wild type Ad5 pIX protein (SEQ ID NO: 1). Even more preferably, the mutation is a substitution mutation of one or more residues corresponding to residues 100, 103, 104, 106, 107, 110, 113, 114, 117 or 121 of the wild type Ad5 pIX protein (SEQ ID NO: 1). Preferred mutations involve the substitution of the aforementioned residues with a proline or a charged residue and most preferably:

the substitution of the leucine in position 114 by a proline (L114P),

the substitution of the valine in position 117 by an aspartic acid (V117D), or

the substitution of the leucine in position 114 by a proline and the substitution of the valine in position 117 by an aspartic acid (L114P-V117D or L-V).

According to a preferred embodiment, such a mutated pIX protein is also modified so as to comprise a binding moiety. As described above, the binding moiety is preferably inserted within the C-terminal part of the mutated pIX protein, preferably after the leucine repeat (e.g. at the C-terminus, between residues 131 and 132 or after residue 127).

In a further aspect, the invention relates to nucleic acid molecules comprising a nucleotide sequence encoding the adenovirus pIX protein of the invention as it is described herein above.

Within the context of the present invention, the term “nucleic acid molecule” defines a polymeric form of any length of deoxyribonucleotides (DNA) or ribonucleotides (RNA). The nucleic acid molecule can be single or double-stranded, linear or circular. It is preferably a double-stranded DNA molecule. It may also comprise modified nucleotides, such as methylated nucleotides or nucleotide analogs (see U.S. Pat. Nos. 5,525,71 1, 4,711,955 or EPA 302 175 as examples of modifications). If present, modifications to the nucleotide structure may be imparted before or after assembly of the polymer (such as by conjugation with a labeling component). The sequence of nucleotides may also be interrupted by non-nucleotide elements. The nucleic acid molecule of the present invention can code for a full length modified pIX protein or for a fragment thereof (e.g. restriction endonuclease-generated and PCR-generated fragments that can be obtained therefrom). The present invention also encompasses synthetic fragments (e.g. produced by oligonucleotide synthesis).

The nucleic acid molecule of the present invention is preferably a vector for cloning or expressing such modified pIX protein. Any type of vector can be used in the context of the present invention, whether of plasmid or viral, integrating or nonintegrating origin. Such vectors are commercially available or described in the literature. Similarly, those skilled in the art are capable of adjusting the regulatory elements required for the expression of the DNA fragment of the invention. Preferably, said vector is an adenoviral vector capable of producing under suitable culturing conditions, virus or virus-like particles bearing at their surface a modified pIX protein according to the present invention (as described hereinafter).

In a preferred embodiment, the nucleotide sequences comprised by the nucleic acid molecule of the invention encodes a fusion of the binding moiety with the native pIX protein or with a mutated pIX protein as described above.

In a further aspect, the present invention relates to an adenoviral vector which comprises the nucleic acid molecule of the invention. The terms “adenoviral genome” and “adenoviral vector” are synonyms and generally refer to the genetic material contained in a virus or virus-like particle, preferably an adenovirus. More specifically, these terms designate a nucleic acid sequence of adenoviral origin comprising at least adenoviral ITR 5′, ITR 3′ and encapsidation (psi) or “packaging” sequence able to promote packaging of said adenoviral genome into an adenoviral particle in order to produce an adenovirus (or virion). Said genome or vector can further comprise all or part of the E1a, E1b, E2a, E2b, E3, E4 adenoviral regions. An adenoviral vector according to the present invention is derived from the genome of a natural or wild-type adenovirus, advantageously a canine, avian or human adenovirus, preferably a human adenovirus type 2, 3, 4, 5 or 7 and, as an absolute preference, a human adenovirus type 5 (Ad5). In this latter case, the deletions of the adenoviral vector according to the invention are indicated by reference to the position of the nucleotides of the Ad5 genome which is specified in the GenBank data bank under the reference M73260 (see FIG. 6 and SEQ ID NO:1).

An adenoviral vector according to the invention is preferably defective for replication, but capable of being replicated and encapsidated in a complementation cell which provides the vector in trans with the product(s) for which it is defective so as to generate an adenoviral particle comprising an adenoviral genome (also termed defective adenovirus) which is incapable of autonomous replication in a host cell but nevertheless infectious, since it has the capacity to deliver the vector to a target cell. However, an adenovirus vector according to the invention can further relate to replication competent vectors (i.e. not defective for replication; Alemany et al., 2000, Nature Biotechnology, 18, 723-727), as well as to conditionally replicative adenoviral vectors (CRAd ; Heise and Kim, 2000, J. of Clinical Investigation, 105, 847-851).

Preferably, the nucleic acid molecule of the invention is placed in the adenoviral genome vector in replacement of the wild-type pIX-encoding gene, using the native pIX promoter to drive expression of said nucleic acid molecule. It is also possible to inactivate the wild-type pIX-encoding gene (e.g. by deletion or mutation) and to insert the nucleic acid molecule of the invention at another (non-native) location in the adenoviral region, either under the control of the native pIX promoter or under the control of an heterologous promoter (e.g. an inducible or constitutive promoter) to make said nucleic acid molecule expressed when desired (e.g. in an appropriate cell during the process of preparation of a virus or virus-like particle, such as the 293 or PER-C6 cell line). The promoters described in connection with the gene of interest (or exogenous sequence) are suitable in this context.

Furthermore, the adenoviral vector of the present invention can be further modified especially to reduce or abolish interaction with the cellular receptors which normally mediate virus attachment and/or entry in the target cells (e.g. interaction between the fiber and the CAR receptor, between penton base and integrins and the like) In this respect, it is known that the host specificity of Ad2 and Ad5 is different from that of Ad3 and Ad7 with respect to CAR-mediated pathway. Thus, it would be advantageous to replace one or more residues of an AdS or Ad2 fiber involved in CAR-binding with equivalent residues originating from an equivalent region of an Ad3 or Ad7 fiber, so as to decrease the ability of said fiber to bind the CAR receptor. By way of illustration, suitable CAR-ablating mutations include those described in WO98/44121, WO01/16344, WO/0138361 and WO00/15823 as well as in Kirby et al. (2000, J. Virol. 74, 2804-2813) and Leissner et al. (2001, Gene Ther. 8, 49-57), with a special preference for the substitution of the serine in position 408 of the Ad5 fiber by a glutamic residue.

The adenoviral fiber can be further modified for example in the shaft region to provide a <<short shafted >> fiber, for example as described in U.S. Pat. No. 5,962,311. By “short-shafted fiber” a fiber is meant whose shaft is shorter than that which is present in a given naturally occurring, i.e., wild-type, adenovirus. For example, a shaft is shorter than that which is present in Ad2 or Ad5. The shaft can be shortened by replacement of a longer fiber with a shorter fiber, which may be of a different serotype. In this regard, the fiber shaft and knob can be of the same serotype or the shaft can be of one serotype and the knob can be of another serotype. For instance, an Ad9 fiber shaft can be used with an Ad2 or Ad5 knob. Preferably, however, the shaft is shortened by deletion of a portion of the shaft, preferably a complete repeat distal to the tail. Preferably, the shaft comprises at least about six repeats, more preferably from about six to about twelve repeats.

In the context of the present invention, an adenoviral vector has as its objective the transfer of an exogenous nucleotide sequence (or gene of insterest) to a target cell and its expression therein. “Exogenous nucleotide sequence” is understood to mean a nucleic acid which comprises at least one coding sequence and, preferably also, regulatory sequences permitting the expression of said coding sequence(s). The exogenous nucleotide sequence and preferably the coding sequence(s) comprised by it are sequences which are normally not present in the genome of an adenovirus. The exogenous nucleotide sequence may be introduced into an adenoviral vector according to the invention by standard techniques of genetic engineering (see e.g. Sambrook and Russell (2001), Molecular Cloning, CSH Press, NY, USA), preferably by homologous recombination as disclosed in EP 742 834, preferentially between the encapsidation region and the 3′ ITR. Such an adenoviral vector comprising an exogenous nucleotide sequence is called “recombinant” as opposed to the “wild type” adenoviral vector or corresponding adenoviral vectors which are modified as described above, but do not contain an exogenous nucleotide sequence.

The term “gene” refers to a nucleic acid comprising a coding sequence that may contain introns, or a fragment thereof, or a cDNA,or a fragment thereof.

Preferably, the exogenous nucleotide sequence comprises a gene suitable for gene therapy, i.e. is therapeutically useful.

Among genes of interest which are preferably usable in the context of the present invention, there may be mentioned:

the genes coding for cytokines such as interferon alpha, interferon gamma, beta-interferon, interleukins;

the genes coding for membrane receptors such as the receptors recognized by pathogenic organisms (viruses, bacteria or parasites), preferably by the HIV virus (human immunodeficiency virus);

the genes coding for coagulation factors such as factor VIII and factor IX;

the gene coding for dystrophin;

the gene coding for insulin;

the genes coding for proteins participating directly or indirectly in cellular ion channels, such as the CFTR (cystic fibrosis transmembrane conductance regulator) protein;

the genes coding for antisense RNAs or proteins capable of inhibiting the activity of a protein produced by a pathogenic gene, present in the genome of a pathogenic organism, or by a cellular gene, the expression of which is deregulated, for example an oncogene;

the genes coding for a protein inhibiting an enzyme activity, such as alpha1—antitripsin or a viral protease inhibitor;

the genes coding for variants of pathogenic proteins which have been mutated so as to impair their biological function, such as, for example, trans-dominant variants of the TAT protein of the HIV virus which are capable of competing with the natural protein for binding to the target sequence, thereby preventing the activation of HIV;

the genes coding for antigenic epitopes in order to increase the host cell's immunity;

the genes coding for major histocompatibility complex classes I and II proteins, as well as the genes coding for the proteins which are inducers of these genes;

the genes coding for cellular enzymes or those produced by pathogenic organisms; and

suicide genes. The TK-HSV-1 suicide gene may be mentioned more especially. The viral TK enzyme displays markedly greater affinity compared to the cellular TK enzyme for certain nucleoside analogues (such as acyclovir or gancyclovir). It converts them to monophosphated molecules, which can themselves be converted by the cellular enzymes to nucleotide precursors, which are toxic. These nucleotide analogues can be incorporated in DNA molecules undergoing synthesis, hence chiefly in the DNA of cells in a state of replication. This incorporation enables dividing cells such as cancer cells to be destroyed specifically.

This list is not restrictive, and other genes of interest may be used in the context of the present invention.

In one embodiment, the gene is a suicide gene encoding a molecule having a directly or indirectly cytotoxic function. By “directly or indirectly” cytotoxic, it is meant that the molecule encoded by the gene may itself be toxic (for example ricin; tumour necrosis factor; interleukin-2; interferon-gamma; ribonuclease; deoxyribonuclease; Pseudomonas exotoxin A) or it may be metabolised to form a toxic product, or it may act on something else to form a toxic product. The sequence of ricin cDNA is disclosed in Lamb et al (1985) Eur. J. Biochem. 148, 265-270 incorporated herein by reference.

For example, it may be desirable to target an exogenous DNA sequence encoding an enzyme using the adenoviral vector of the invention or a virus or virus-like particle containing it, the enzyme being one that converts a relatively non-toxic prodrug to a toxic drug. The enzyme cytosine deaminase converts 5-fluorocytosine (5FC) to 5-fluorouracil (5FU) (Mullen et al (1922) PNAS 89, 33); the herpes simplex enzyme thymidine kinase sensitises cells to treatment with the antiviral agent ganciclovir (GCV) or aciclovir (Moolten (1986) Cancer Res. 46, 5276; Ezzedine et al (1991) New Biol 3, 608). The cytosine deaminase of any organism, for example E. coli (EP 402 108) or Saccharomyces cerevisiae (Erbs et al., 1997, Curr. Gennet. 31, 1-6; W093/01281), maybe used.

Thus, in a preferred embodiment of the invention, the gene encodes a cytosine deaminase. In accordance with this embodiment, the patient may be concomitantly given 5FC and a virus or virus-like particle expressing cytokine deaminase. By “concomitantly”, it is meant that 5FC is administered at such a time, in relation to the transformation of the target cells, such as tumour cells, that 5FC is converted into 5FU in the target cells by the cytosine deaminase expressed from the said gene. A dosage of approximately 0.001 to 100.0 mg 5C/kg body weight/day, preferably 0.1 to 10.0 mg/kg/day is suitable.

The use of a combination of suicide gene products, e.g. cytosine deaminase and uracil phosphoribosyl transferase activity can also be envisaged in the context of the invention. Suitable genes encoding uracil phosphoribosyl transferase include those from E. coli (Anderson et al., 1992, Eur. J. Biochem. 204, 51-56) and Saccharomyces cerevisiae (Kern et al., 1990, Gene 88, 149-157). Advantageously, the exogenous DNA sequence in use in the present invention encodes a polypeptide having both cytosine deaminase and uracil phosphoribosyl transferase activities. Cytosine deaminase deaminates the 5-FC analog, therby forming 5-fluorouracil (5-FU), which is highly cytotoxic when it is converted into 5-fluoro-UMP by uracil phosphoribosyl transferase action. Such a polypeptide is described for example in WO96/16183 and WO99/54481.

Components, such as 5FC, which are converted from a relatively non-toxic form into a cytotoxic form by the action of an enzyme are termed “pro-drugs”.

Other examples of pro-drug/enzyme combinations include those disclosed by Bagshawe et al (WO88/07378), namely various alkylating agents and the Pseudomonas spp. CPG2 enzyme, and those disclosed by Epenetos & Rowlinson-Busza (WO 91/11201), namely cyanogenic pro-drugs (for example amygdalin) and plant-derived beta-glucosidases.

Enzymes that are useful in this embodiment of the invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (see, e.g. R. J. Massey, Nature, 328, pp. 457-458 (1987)).

Similarly, the prodrugs include, but are not limited to, the above-listed prodrugs, e.g., phosphate-containing prodrugs, thiophosphate-containing prodrugs, sulfate-containing prodrugs,peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted by the enzyme from the conjugate into the more active, cytotoxic free drug. Examples of cytotoxic drugs that can be derivatized into a prodrug form for use in this invention include, but are not limited to, etoposide, teniposide, adriamycin, daunomycin, carminomycin, aminopterin, dactinomycin, mitomycins, cis-platinum and cis-platinum analogues, bleomycins, esperamicins (see U.S. Pat. No. 4,675,187), 5-fluorouracil, melphalan and other related nitrogen mustards.

In a further embodiment, the gene delivered to the target cell encodes a ribozyme capable of cleaving targeted RNA or DNA. The targeted RNA or DNA to be cleaved may be RNA or DNA which is essential to the function of the cell and cleavage thereof results in cell death or the RNA or DNA to be cleaved may be RNA or DNA which encodes an undesirable protein, for example an oncogene product, and cleavage of this RNA or DNA may prevent the cell from becoming cancerous.

In a still further embodiment, the gene delivered to the target cell encodes an antisense RNA.

The term “antisense RNA” means an RNA molecule which hybridises to, and interferes with the expression of an mRNA molecule encoding a protein or to another RNA molecule within the cell such as pre-mRNA or tRNA or rRNA, or hybridises to, and interferes with the expression of a gene.

Furthermore, a gene delivered to the target cell may also encode other species of RNA molecules that are capable of influencing gene expression of the cell such as RNA molecules that may exert an RNA interference (RNAi) or a co-suppression effect.

Conveniently, a gene expressing an antisense RNA may be constructed by inserting a coding sequence encoding a protein adjacent to a promoter in the appropriate orientation such that the transcribed RNA is complementary to the target mRNA. Suitably, the antisense RNA blocks expression of undesirable polypeptides such as oncogenes, for example ras, bcl, src or tumour suppressor genes such as p53 and Rb.

It will be further appreciated that DNA sequences suitable for being expressed as an antisense RNA may be readily derived from publicly accessible databases such as GenBank and EMBL.

In another embodiment of the invention, the gene may replace the function of a defective gene in the target cell. There are several thousand inherited genetic diseases of mammals, including humans, that are caused by defective genes. Examples of such genetic diseases include cystic fibrosis, which is known to be caused by a mutation in the CFTR gene; Duchenne muscular dystrophy, which is known to be caused by a mutation in the dystrophin gene; sickle cell disease, which is known to be caused by a mutation in the HbA gene. Many types of cancer are caused by defective genes, especially protooncogenes, and tumour-suppressor genes that have undergone mutation.

Thus, it is preferred that an adenoviral vector of the invention or a virus or virus-like particle containing it, which may be useful in the treatment of cystic fibrosis, contains a functional CFTR gene to replace the function of the defective CFTR gene. Similarly, it is preferred that an adenoviral vector of the invention, which may be useful in the treatment of cancer, contains a functional protooncogene or tumour-suppressor gene to replace the function of the defective protooncogene or tumour-suppressor gene.

Examples of protooncogenes are ras, src, bcl and so on; examples of tumour-suppressor genes are p53 and Rb.

It will be appreciated that the gene will be introduced into a convenient place within the adenoviral vector and will contain a promoter and/or enhancer element to drive its expression.

It is preferred that the promoter and/or enhancer element is selective for the cells to be targeted. Some examples of tissue or tumour specific promoters are given below but new ones are being discovered all of the time which will be useful in this embodiment of the invention.

For example, the mucin gene, MUC1, contains 5′ flanking sequences which are able to direct expression selectively to breast and pancreatic cell lines, but not to non-epithelial cell lines as it is taught in WO 91/09867.

In the context of the present invention, an exogenous nucleotide sequence can consist of one or more gene(s) of interest, and preferably is of therapeutic interest. In the context of the present invention, a gene of interest can for example code for an antisense RNA, or for an MRNA which will then be translated into a protein of interest. A gene of interest can be of genomic type, of complementary DNA (cDNA) type or of mixed type (e.g. a minigene, in which at least one intron is deleted). It can code for a mature protein, a precursor of a mature protein, in particular a precursor intended to be secreted and accordingly comprises a corresponding signal peptide, a chimeric protein originating from the fusion of sequences of diverse origins, or a mutant of a natural protein displaying improved or modified biological properties. Such a mutant may be obtained by mutation, deletion, substitution and/or addition of one or more nucleotide(s) of the gene coding for the natural protein. Other genes of interest may for instance encode a ribozyme or an RNA interference (RNAi) construct, capable of inhibiting gene expression of an endogenous gene in the transfected cell. Means and methods for constructing ribozymes and RNAi constructs are known to the person skilled in the art and are well documented in the pertinent literature.

A gene of interest may be placed under the control of elements suitable for its expression the target cell. “Suitable elements” are understood to mean the set of elements needed for the transcription of the gene of interest into RNA (e.g. antisense RNA or mRNA) and for the translation of an mRNA into protein. Among the elements needed for transcription, the promoter assumes special importance. It can be a constitutive promoter or a regulatable promoter, and can be isolated from any gene of eukaryotic or viral origin, and even adenoviral origin. Alternatively, it can be the natural promoter of the gene of interest in question. Generally speaking, a promoter used in the present invention may be modified so as to contain regulatory sequences. As examples, a gene of interest in use in the present invention may be placed under the control of the promoter of one of the immunoglobulin genes when it is desired to target its transfer to lymphocytic host cells. There may also be mentioned the TK-HSV-1 (herpesvirus, type 1 thymidine kinase) gene promoter or alternatively the adenoviral MLP promoter, in particular from human adenovirus type 2, permitting expression in a large number of cell types.

In a preferred embodiment, the transcriptional control sequence comprises a promoter element. Preferably, one would use a high expression promoter. Said promoter may be for example selected from the group consisting of viral promoters and tissue- or cell type-specific promoters such as muscle specific promoters, or a combination thereof. Examples of such viral promoters are the SV40 early and late promoters, the adenovirus major late promoter, the Rous Sarcoma Virus (RSV) promoter, the Cytomegalovirus (CMV) immediate-early promoter, the herpes simplex virus (HSV) promoter, the MPSV promoter, the 7.5 k promoter, the vaccinia promoter and the Major-intermediate-early (MIE) promoter. Examples of muscle specific promoters are the smooth muscle 22 (SM22) promoter, the myosin light chain promoter, the myosin heavy chain promoter, the skeletal alpha-actin promoter and the dystrophin promoter. The Cytomegalovirus (CMV) immediate-early promoter is however preferred. The natural promoter of the beta-interferon encoding sequence may also be used (U.S. Pat. No. 4,738,931). The polynucleotide sequence of the promoter can be a naturally occurring promoter sequence isolated from biological nucleic acid material or chemically synthesized. The promoter sequence can also be artificially constructed by assembling elements previously screened for transcriptional activity leading to potencies which can exceed those of naturally occurring ones (Li et al., 1999, Nature Biotech., 17, 241-245). The expression cassette (comprising coding sequence and promoter) can be constructed using routine cloning techniques known to persons skilled in the art (for example, see Sambrook et al., 2001, supra). In still another aspect of the invention, the transcriptional control sequence further comprises at least one enhancer element. The term “enhancer” refers to a regulatory element which activates transcription in a position and orientation independent way. Several enhancer elements have been identified to date in many genes. For example, the enhancer element may be a myosin light chain enhancer. More preferably, the enhancer used in the expression cassette of the present invention is of vertebrate origin, more preferably of mammalian origin. The rat myosin light chain 1/3 enhancer (Donoghue et al., 1988, Gene & Dev., 2, 1779-1790) is especially useful. The enhancer element operably linked to the promoter may be localized either upstream or downstream of said promoter and may be used in either orientation. According to a preferred embodiment, the transcriptional control sequence comprises several enhancer sequences, the sequences of which are identical or selected independently of one another. Preferably, the transcriptional control sequence further comprises at least one sequence ensuring the polyadenylation of the transcribed RNA molecules. Such a sequence may be selected from the group consisting of the bGH (bovine growth hormone) polyadenylation signal (EP 173552), the SV40 polyadenylation signal and the globine polyadenylation signal, and is generally located at the 3′-end of the sequence encoding the protein, e.g. beta-interferon or RNA to be expressed.

Moreover, according to another embodiment of the invention, an adenoviral vector according to the invention can, preferably in addition to the aforementioned genes, comprise a non-therapeutic gene coding for a protein which trans-activates non-adenoviral transcription. Naturally, the gene(s) of the E1A region coding for a trans-activating protein, the expression of which would run the risk of rendering the adenovirus non-defective, will be avoided.

In addition, the present invention relates to a method for producing a virus or virus-like particle comprising the steps of

(a) transforming a suitable host cell with an adenoviral vector of the invention as described above;

(b) culturing the transformed cell line under conditions suitable to allow formation of a virus or virus-like particle from said adenoviral vector; and

(c) recovering the virus or virus-like particle formed in step (b) from the culture.

The adenoviral vector of the invention can be transformed into the cell in accordance with known techniques, such as microinjection into the cell nucleus (Capechi et al., 1980, Cell 22, 479-488), transfection for example with CaPO₄ (Chen and Okayama, 1987, Mol. Cell Biol. 7, 2745-2752), electroporation (Chu et al., 1987, Nucleic Acid Res. 15, 1311-1326), lipofection/liposome fusion (Felgner et al., 1987, Proc. Natl. Acad. Sci. U.S. Pat. No.A 84, 7413-7417), particle bombardment (Yang et al., 1990, Proc. Natl. Acad. Sci. U.S. Pat. No.A 87, 9568-9572), gene guns, infection (e.g. with a virus containing said adenoviral vector), direct DNA injection (Acsadi et al., 1991, Nature 352, 815-818), microprojectile bombardment (Williams et al., 1991, Proc. Natl. Acad. Sci. U.S. Pat. No. 88, 2726-2730), or the like.

With respect to cell line, both prokaryotic and eukaryotic cells may be employed, which include bacteria yeast, plants and animals, including human cells. Preferably, the adenoviral vector is replication-defective and said suitable host cell complements at least one defective function of said adenoviral vector, eventually in combination with a helper virus. The cell lines 293 (Graham et al., 1977, J. Gen. Virol. 36, 59-72) and PERC6 (Fallaux et al., 1998, Human Gene Therapy 9, 1909-1917) are commonly used to complement the E1 function. Other cell lines have been engineered to complement doubly defective vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033 EP919627 and W097/04119).

According to one embodiment, the genome of the adenoviral vector lacks all or part of the sequence encoding a pIX (either wild type or modified), and the method of the invention employs preferably a host cell engineered to express a modified adenoviral pIX protein of the invention, and preferably a pIX protein which has been genetically modified to express a binding moiety within its C-terminal part. Such a cell line comprises either in a form integrated into the genome or in episome form a nucleic acid molecule encoding the modified adenoviral pIX protein of the invention. Of course, the nucleic acid molecule is placed under the control of appropriate translational and/or transcriptional regulatory elements to allow production of the modified pIX protein in said cell line. Preferably, this cell line is further capable of complementing one or more adenoviral functions selected from the group consisting of the functions encoded by the E1, E2, E4, LI, L2, L3, L4, L5 regions or any combination thereof. It is preferably produced from the 293 cell line or from the PER-C6 cell line complementing the E1 function by transfecting an expression vector encoding the sequence encoding the modified pIX protein.

The virus or virus-like particle can be recovered from the culture supernatant but also from the cells after conventional lysis techniques (mechanical, enzymatic, freeze and thawing cycles) and optionally further purified according to standard techniques (e.g. ultracentrifugation, chromatography as described in WO96/27677, WO98/00524 WO98/26048 and WO00/50573).

In a further embodiment, the invention relates to a method for producing a virus or virus-like particle comprising the steps of

(a) transforming a suitable host cell with an adenoviral vector which encodes a wild-type pIX protein;

(b) modifying the coding sequence for the pIX protein in the adenoviral vector so that the encoded pIX protein is one according to the present invention;

(c) culturing the host cell under conditions suitable to allow formation of a virus or virus-like particle from the adenoviral vector of step (b); and

(d) recovering the virus or virus-like particle formed in step (c) from the culture.

Preferably, this method includes the production in cell culture of a virus or virus-like particle which has been genetically modified to express a binding moiety on its surface, whereby this binding moiety is comprised by the modified pIX protein of the invention. The virus or virus-like particle is grown in its host prior to modification, but once the modification that alters the binding specificity is made, the virus or virus-like particle is grown in the target cell. Thus, for example in the case where the binding moiety recognises a breast tumour cell antigen, the modified virus or virus-like particle is grown in breast tumour cell culture expressing that antigen.

In a further embodiment, the present invention relates to viruses or virus-like particles comprising the above-described adenovirus pIX protein, the nucleic acid molecule or the adenoviral vector of the invention or are obtainable by a method for producing a virus or virus-like particle as mentioned hereinabove.

Advantageously, the virus or virus-like particle of the invention is furthermore substantially incapable of binding its host cell, i.e. incapable of binding the natural host cell of the wild-type virus or virus-like particle from which said virus or virus-like particle is derived. The term “derived” means in this context that the wild-type virus or virus-like particle structurally corresponds to the virus or virus-like particle of this embodiment of the invention, except for modifications rendering it suitable for gene delivery, for instance by deleting non-essential parts of the viral genome and/or inserting a heterologous sequence to be expressed, and for the modification of the pIX protein and its coding sequence as described herein. By “a virus or virus-like particle substantially incapable of binding its host cell”, a modified virus is meant that has no more than 20%, preferably no more than 10%, more preferably no more than 5%, and even more preferably no more than 1% of the binding affinity of the unmodified virus for the host cell. This lack of binding can be obtained for example by modifying the fiber protein as discussed above and disclosed in the prior art (see for example U.S. Pat. Nos. 5,756,086, 5,885,808, EP 991 763, PCT FR00/01559 or PCT FR00/03263).

In accordance with a preferred embodiment of the present invention, the virus or virus-like particle comprises a modified binding specificity conferred by a pIX moiety modified in the sense that it comprises a binding moiety in order to allow the virus or virus-like particle to bind to a target cell. Preferably, said virus or virus-like particle is furthermore substantially incapable of binding its host cell.

In order to differentiate the cells for which the virus or virus-like particle of the invention shows specificity in respect to the capacity to infect, a distinction is made herein between “host cell” and “target cell”. However, further below, for instance in those parts of the description where the function of an exogenous nucleotide sequence in the target cell is described, this distinction may not apply. Accordingly, in contrast to “target cell”, the term “host cell” refers to the cells to which an unmodified, naive virus can bind by using its receptor-like molecule and the cognate receptor-like molecule on the surface of the cell. The term “target cell” refers to cells to which the modified virus can bind by using its binding moiety. In some circumstances, in the context of this aspect of the invention, such as when the binding moiety recognises an entity on the host cell which is not the cognate receptor-like molecule, then the host cell may be the target cell.

In a preferred embodiment of the invention, the target cell is a eukaryotic, especially mammalian cell, and it is expected that the invention will find uses in the areas of gene therapy and cancer treatment.

It is also preferred that the virus or virus-like particle is “replication-defective”. By “replication defective”, a virus is meant whose genetic material has been manipulated so that it cannot divide or proliferate in the cell it infects.

The binding moiety of the virus or virus-like particle of the invention provides the target cell binding specificity.

In one preferred embodiment, the virus or virus-like particle is an adenovirus. Preferentially, in such a virus or virus-like particle, the E1B gene is substantially deleted or modified so that its gene product no longer interacts with the E1A protein. E1A protein stimulates apoptosis but normally its action is inhibited by E1B. Conveniently, the E1B gene is inactivated by insertion; preferably a cytotoxic gene, as defined below, is inserted at or near the E1B gene.

E1, E3 and a site upstream of E4 may be used as sites for insertion of foreign DNA sequences in the generation of recombinant adenoviruses (for example see Berkner and Sharp (1984) Nucl. Acids Res. 12, 1925-1941; Chanda et al (1990) Virology 175, 535-547; Haj-Ahmad and Graham (1986) J. Virol. 57, 267-274; Saito et al (1985) J. Virol. 54, 711-719; all incorporated herein by reference). Since the upper size limit for DNA molecules that can be packaged into adenovirus particles is approximately 105% of the wild-type genome, only about 2 kb of extra DNA can be inserted without compensating deletions of viral DNA. Although E1 is essential for virus replication in cell culture, foreign DNA can be substituted for E1 sequences when the virus is grown in 293 or PERC6 cells which are transformed by Ad5 DNA and constitutively express E1 (Graham et al (1977) J. Gen. Virol. 36, 59-72 and U.S. Pat. No. 6,033,908, incorporated herein by reference). Several vectors having 1.9 kb deleted from E3 of Ad5 have been constructed without interfering with virus replication in cell culture (reviewed by Graham and Prevec (1992) in “Vaccines: New Approaches to Immunological Problems” R. W. Ellis (Ed.), Butterworth-Heinemann, Boston, Mass., pages 364-390, incorporated herein by reference). Such vectors allow for insertion of up to 4 kb of foreign DNA. Recombinant adenoviruses containing inserts in E3 replicate in all Ad-permissive cell lines and a number of adenovirus vectors containing E3 inserts have been shown to express foreign genes efficiently both in vitro and in vivo. As mentioned above, the adenovirus or adenovirus-like particle can also be modified (e.g. in the fiber, penton base and/or hexon), so as to substantially reduce or abolish the binding to the cellular receptors which normally mediate attachement and entry of a wild-type adenovirus and adenovirus-like particles to its host cell.

Substantially replication-defective adenoviruses may be made by creating a deficiency of the E1A protein. Suitably this is achieved by deleting the E1A gene or by making mutations within the E1A gene that prevent expression of the E1A protein. Examples of suitable mutations are deletions within the E1A coding region; nonsense mutations; and frame shift mutations.

The viruses or virus-like particles of the invention can be propagated in a complementation cell line providing in trans the missing function which is essential to viral replication (encoded by E1, E2 and/or E4 regions). Widely used complementation line are the embryonic kidney line 293 (Graham et al.,1977, J. Gen. Virol., 36, 59-72), 911 cells (deposited under no 95062101 at the ECACC—Fallaux et al. (1996) Hum. Gene Ther. 7, 215-222) or the embryonic retinal line PERC6 (ECACC N°. 96022940) cells. PER.C6 cells is the abbreviation of PGK-E1 Retinoblasts. C6 is the clone number. PGK-E1 reflects that the adenovirus type 5 E1A region in the construct is driven by the human PGK promoter. PER cells are Human Embryo Retinoblasts transformed with E1 sequences (nt 459-3510) of human adenovirus type 5 (WO 97/00326).

Cells useful for propagating the virus or virus-like particle of the invention may additionally be derivatives of existing cell lines, e.g., from 293 or PER.C6 cell lines. Such derivative cells should express the genes necessary to complement in trans deletions in an adenoviral genome or should support replication of an otherwise defective adenoviral vector. Such cells may for example express the E1, E2, E4, and/or late functions (see for example EP 919 627, U.S. Pat. Nos. 6,040,174, 6,133,028, 6,033,908 or U.S. Pat. No. 5,994,128).

For the purposes of the present invention, the term “deletion” or “lacking” refers to the elimination of at least one nucleotide in the target region, and the deletion can naturally be continuous or discontinuous. Generally speaking, such a deletion results in a measurable change : for example, the deletion results in impairing or improving the function of the product encoded by the genetic material bearing the deletion compared with the corresponding genetic material not bearing the deletion. For example, a deletion in the E1 region results in lack of adenoviral replication. These terms are widely used by those skilled in the art. “All or part” of the regions of the adenoviral genome may be deleted which means that either the whole or only a portion of the region in question is removed. Deletions are preferred which prevent the production of at least one expression product encoded by the said region. Hence they may lie in a coding region or a regulatory region such as the promoter region, and may affect at least one nucleotide so as to destroy the reading frame of a gene or render a promoter region non-functional. The deletions in question may also comprise partial deletions of one or more genes of the said region or of the whole of the region.

The term “virus or virus-like particle” as used herein is synonymous with “adenoviral capsid” which, in turn, is a general term designating both “adenoviral particle” and “adenoviral pseudo particle”. “Adenoviral particle” concerns an “adenoviral genome” (recombinant or wild type) associated with viral polypeptides forming what is usually called an adenovirus, or forming a complex where the nucleic acid, while being associated with the viral polypeptides, is not included into a viral element such as a viral capsid (see U.S. Pat. No. 5,928,944 and WO 9521259). On the opposite, “non-naked” means that said nucleic acid may be associated (i) with viral polypeptides forming what is usually called a virus (adenovirus, retrovirus, poxvirus, etc . . . ) or forming a complex where the nucleic acid while being associated with viral polypeptides is not included into a viral element such as a viral capsid (see U.S. Pat. No. 5,928,944 and WO 9521259). An adenoviral particle according to the invention may be prepared by passage in any complementation line providing in trans the functions for which an adenoviral vector according to the invention is defective, for example line 293 of the prior art. These preparation techniques are known to a person skilled in the art (Graham and Prevec, 1991, Methods in Molecular Biology, vol. 7, 109-128, Ed: E. J. Murey, The Human Press Inc.).

Furthermore, the virus or virus-like particle of the invention can be of use for transferring non-viral macromolecules into a target cell. There are two means by which such transfer can be effected. First, the adenoviral vector be employed to transfer non-viral macromolecules packaged within the adenoviral vector either in place of, or in addition to, normal adenoviral components (Berkner, K. L., BioTechniques, 6, 606-629 (1988)). For example, the genome of the adenovirus can be modified to incorporate an exogenous nucleotide sequence. The recombinant adenovirus is then packaged to constitute an infectious virus capable of entering cells and transferring the exogenous nucleotide sequence to the nucleus (Rosenfeld et al., Science, 252, 431-434 (1991); Rosenfeld et al., Cell, 68, 143-155 (1992); Quantin et al., Proc. Natl. Acad. Sci., 89, 2581-2584 (1992); Berkner, K. L., BioTechniques, 6, 606-629 (1988)).

Second, the virus or virus-like particle can be employed to mediate the transfer of non-viral macromolecules either linked to the surface of the adenoviral vector or, in a “bystander” process where the macromolecule is cointemalized, taken along as cargo in the adenoviral receptor-endosome complex (Otero et al., Virology, 160, 75-80 (1987); FitzGerald et al., Cell, 32, 607-617 (1983); Seth et al., Mol. Cell Biol., 4, 1528-1533 (1984); Yoshimura, Cell Struct. Funct., 10, 391-404 (1985); Defer et al., J. Virol., 64, 3661-3673 (1990)).

The mechanism by which an adenovirus may augment internalization of non-viral biologic material is believed to be by increasing the permeability of the target cell plasma membrane (Otero et al., Virology, 160, 75-80 (1987)) or, more likely, by cointernalization of the exogenous biologic material as an “innocent bystander” when the adenovirus-receptor complexes cluster on the membrane and are internalized (FitzGerald et al., Cell, 32, 607-617 (1983); Seth et al., Mol. Cell Biol., 4, 1528-1533 (1984); Yoshimura, Cell Struct. Funct., 10, 391-404 (1985); Otero et al., Virology, 160, 75-80 (1987); Defer et al., J. Virol., 64, 3661-3673 (1990)). These processes are not adenovirus-specific, as similar phenomena have been observed with other non-enveloped viruses such as picornavirus (Femandez-Puentes et al., Cell, 20, 769-775 (1980); Otero et al., Virology, 160, 75-80 (1987); Carrasco, Virology, 113, 623-629 (1981)), as well as enveloped viruses including paramyxovirus, rhabdovirus, poxvirus, and togavirus (Fernandez-Puentes et al., Cell, 20, 769-775 (1980); Otero et al., Virology, 160, 75-80 (1987); Yamaizumi et al., Virology, 95, 216-221 (1979); Carrasco et al., Virology, 117, 62-69 (1982)).

Most of the research attention on virus-mediated cointemalization of macromolecules has been focused on cointernalization of proteins, including toxins and various reporter proteins (Ferna-Puentes et al., Cell, 20, 769-775 (1980); FitzGerald et al., Cell, 32, 607-617 (1983); Seth et al., Mol. Cell Biol., 4, 1528-1533 (1984); Otero et al., Virology, 160, 75-80 (1987); Defer et al., J. Virol., 64, 3661-3673 (1990); Carrasco, Virology, 113, 623-629 (1981); Yamaizumi et al., Virology, 95, 216-221 (1979); Carrasco et al., Virology, 117, 62-69 (1982)). The concept that cointemalization might be employed for adenovirus-mediated transfer of nucleic acids was suggested, but not evaluated, by Otero and Carrasco (Otero et al., Virology, 160, 75-80 (1987)). In fact, the more recent approaches with respect to the transfer of nucleic acids using an adenovirus have centered on nucleic acid transfer by attachment of the nucleic acid to molecules capable of effecting its entry into the cell. For instance, in one approach, the nucleic acid may be part of a polylysine-glycoprotein carrier complex capable of binding a particular cell surface receptor, or is complexed with a nonspecific ligand such as a charged polypeptide (Rosenfeld et al., Science, 252, 431-434 (1991); Curiel et al., Proc. Natl. Acad. Sci., 88, 8850-8854 (1991); Rosenfeld et al., Cell, 68, 143-155 (1992); Quantin et al., Proc. Natl. Acad. Sci., 89, 2581-2584 (1992); Curiel et al., Hum. Gene Therapy, 3, 147-154 (1992)); Cotten et al., Proc. Natl. Acad. Sci., 89, 6094-098 (1992); Cotten et al., J. Virology, 67, 3777-3785 (1993)). In a more recent approach, the nucleic acid may be attached to the outside of an adenoviral capsid by means of conjugation of the nucleic acid through a polylysine residue to an antibody having affinity to an adenoviral capsid protein (Curiel et al., Human Gene Ther., 3, 147-154 (1992)). Thus, despite this early suggestion by Otero et al., researchers have clearly perceived a lack of feasibility of using adenovirus-driven RME for the transfer of nucleic acids.

Moreover, the uses of the virus or virus-like particle for transferring nucleic acids are limited by the specific receptor to the ligand employed, i.e. the specific receptor must be present on the cell surface for transfection to be accomplished. Additionally, it was discovered recently that better transfection results are obtained when the DNA is not physically attached to any molecule upon introduction into the cell (Wolff et al., Science, 247, 1465 (1990); Acsadi et al., Nature, 352, 815 (1991)). This finding underscores the restrictive nature of current approaches of adenovirus-mediated transfer of DNA to the cell, which require the attachment of DNA to one or more other moieties for cell transfection.

The invention also relates to a eukaryotic host cell comprising an adenovirus pIX protein, nucleic acid molecule or adenoviral vector according to the invention or being infected with a virus or virus-like particle of the invention. Said cell is advantageously a mammalian cell, and preferably a human cell, and can comprise said vector in integrated form in the genome, or preferably in non-integrated (episome) form.

For the purpose of the invention, the term “eukaryotic host cell” should be understood broadly without any limitation concerning particular organization in tissue, organ, etc or isolated cells of a mammalian (preferably a human). Such cells may be unique type of cells or a group of different types of cells and encompass cultured cell lines, primary cells and proliferative cells from mammalian origin, with a special preference for human origin. Suitable host cells include but are not limited to hematopoïetic cells (totipotent, stem cells, leukocytes, lymphocytes, monocytes, macrophages, APC, dendritic cells , non-human cells and the like), pulmonary cells, tracheal cells, hepatic cells, epithelial cells, endothelial cells, muscle cells (e.g. skeletal muscle, cardiac muscle or smooth muscle), fibroblasts. The present invention also relates to a complementation cell line suitable for producing the virus or virus-like particle of the invention or for applying the method for producing such a virus or virus-like particle as described above.

Such a complementation line may contain a complementation element comprising, in particular, a portion of the E1 region of the genome of an adenovirus with the exception of the 5′ ITR; said complementation element being capable of complementing in trans a defective adenoviral vector and being integrated in the genome of said complementation line or inserted into an expression vector. In particular, the eukaryotic host cell of the invention is a cell of a complementation line, in particular when the adenoviral vector contained in the host cell is defective.

In the context of the present invention, the term “complementation line” refers to a eukaryotic cell capable of providing in trans the function(s) for which an adenoviral vector is defective. In other words, it is capable of producing the protein or proteins needed for the replication and encapsidation of said adenoviral vector, early and/or late proteins which it cannot itself produce and which are needed for building a viral particle. Naturally, said portion may be modified by mutation, deletion and/or addition of nucleotides, as long as these modifications do not impair its capacity for complementation. Thus, for example an adenoviral vector which is defective for the E1 function will have to be propagated in a complementation line for E1 (i.e. capable of providing in trans the protein or set of proteins encoded by the E1 region which the vector cannot produce), a vector which is defective for the E1 and E4 functions will be propagated in a complementation line for E1 and E4 (providing the necessary proteins encoded by the E1 and E4 regions), and lastly a vector which is defective for the E1, E2 and E4 functions will be propagated in a complementation line for the three functions. As mentioned in the introduction, the E3 region is non-essential, and does not need to be specifically complemented.

A complementation line according to the invention may be derived either from an immortalized cell line capable of dividing indefinitely, or from a primary line. In accordance with the objectives pursued by the present invention, a complementation line according to the invention is useful for the encapsidation of any defective adenoviral vector, and especially a defective adenoviral vector according to the invention.

The present invention further relates to a pharmaceutical composition comprising as therapeutic and/or prophylactic agent an adenoviral vector, a virus or virus-like particle, a eukaryotic host cell or a complementation cell line according to the invention, in combination with a vehicle or carrier which is acceptable from a pharmaceutical standpoint. It is preferred that said agent is capable of expressing a therapeutically useful gene such as those enumerated above.

The pharmaceutical composition of the invention can be administered by any suitable route. Administration into vertebrate target tissues, and more specifically into the muscle, can be performed by different delivery routes (systemic delivery and targeted delivery). According to the present invention, the pharmaceutical composition is preferably administered into skeletal muscle, however administration can also occur in other tissues of the vertebrate body for instance including those of non skeletal muscle, skin, brain, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor. Similarly, in order to direct expression of the therapeutically useful gene to the intended site of action, the nucleic acid can be associated with targeting molecules which are capable to direct its uptake into targeted cells. Gene therapy literature provides many mechanisms for efficient and targeted delivery and expression of genetic information within the cells of a living organism. Administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol routes. Injection, and specifically intratumoral, intravenous or intramuscular injection, is preferred.

The virus or virus-like particles of the invention may be administered in any suitable way, usually parenterally, for example intravenously, intraperitoneally or intravesically, in standard sterile, non-pyrogenic formulations of diluents and carriers, for example isotonic saline (when administered intravenously).

The pharmaceutical composition can be designed or used for repeated administrations to the patient without major risk of the administered pharmaceutical composition to induce a significant immune reaction. Administration may be by single or repeated dose, once or several times after a certain period of time. Repeated administration allows a reduction in the dose of the pharmaceutical composition administered at a single time. The route of administration and the appropriate dose vary in function of several parameters, for example the individual patient, the side effects of the disease, or the albumin level before treatment.

The administered volume preferably varies from about 10 μl to 500 ml, most preferably from about 1001 μl to 100 ml. The administered volume can be adapted depending on the administration route, the treated patient and the patient's weight.

The pharmaceutical composition according to the invention is intended especially for the preventive or curative treatment of disorders such as: genetic disorders such as hemophilia, cystic fibrosis or Duchene's and Becker type myopathies, cancers such as those induced by oncogenes or viruses, retroviral diseases such as AIDS (acquired immunodeficiency syndrome resulting from HIV infection), and recurrent viral diseases such as herpesvirus-induced infections. The composition of the present invention is particularly intended for the preventive or curative treatment of disorders, conditions or diseases associated with cancer. The term “cancer” encompasses any cancerous conditions including diffuse or localized tumors, metastasis, cancerous polyps and preneoplastic lesions (e.g. dysplasies) as well as diseases which result from unwanted cell proliferation. A variety of tumors may be selected for treatment in accordance with the composition of the invention. In general, solid tumors are preferred, although leukemias and lymphomas may also be treated especially if they have developed a solid mass, or if suitable tumor associated markers exist such that the tumor cells can be physically separated from nonpathogenic normal cells. For example, acute lymphocytic leukemia cells may be sorted from other lymphocytes with the leukemia specific marker “CALLA”. Cancers which are contemplated in the context of the invention include without limitation glioblastoma, sarcoma, melanomas, mastocytoma, carcinomas (e.g. colorectal and renal cell carcinomas) as well as breast, prostate, testicular, ovarian, cervix (in particular, those induced by a papilloma virus), lung (e.g. lung carcinomas including large cell, small cell, squamous and adeno-carcinomas), kidney, bladder, liver, colon, rectum, pancreas, stomach, esophagus, larynx, brain, throat, skin, central nervous system, blood (lymphomas, leukemia, etc.), bone, etc cancers.

A pharmaceutical composition according to the invention may be manufactured in a conventional manner. In particular, a therapeutically effective amount of a therapeutic or prophylactic agent is combined with a vehicle such as a diluent. A composition according to the invention may be administered by aerosol or via any conventional route in use in the field of the art, especially via the oral, subcutaneous, intramuscular, intravenous, intraperitoneal, intrapulmonary or intratracheal route. The administration may take place in a single dose or a dose repeated one or more times after a certain time interval. The appropriate administration route and dosage vary in accordance with various parameters, for example with the individual being treated or the disorder to be treated, or alternatively with the gene(s) of interest to be transferred. Generally speaking, a pharmaceutical composition according to the invention comprises a dose of adenovirus according to the invention of between 10⁴ and 10¹⁴, advantageously 10⁵ and 10¹³ and preferably 10⁶ and 10¹¹. A pharmaceutical composition, especially one used for prophylactic purposes, can comprise, in addition, an adjuvant which is acceptable from a pharmaceutical standpoint.

The invention also encompasses a method of treatment, according to which a therapeutically effective amount of an adenoviral vector, a virus or virus-like particle, a eukaryotic host cell or a complementation cell line according to the invention is administered to a patient requiring such treatment. Preferably, said treatment is by gene therapy which may be in vivo or ex vivo gene therapy. Accordingly, the invention relates to the use of an adenoviral vector, a virus or virus-like particle, a eukaryotic host cell or a complementation cell line according to the invention for the preparation of a pharmaceutical composition for prophylaxis, treatment and/or vaccination of a patient in need of such prophylaxis, treatment and/or vaccination. Preferably, said adenoviral vector, virus, or virus-like particle, host cell or complementation cell line is capable of expressing a therapeutically useful gene.

In a preferred embodiment, the method according to the present invention involves administration into a fluid vessel, such as for example a blood vessel or a lymph vessel, and can be advantageously improved by combining injection in an afferent and/or efferent fluid vessel with increases of permeability of said vessel. In a particularly preferred embodiment, said increases is obtained by increasing hydrostatic pressure (i.e. by obstructing outflow and/or inflow), osmotic pressure (with hypertonic solution) and/or by introducing a biologically active molecule (e.g. histamine into administered composition) (see, e.g., WO 98/58542).

In addition to the prophylactic and/or therapeutical agents, pharmaceutical compositions of the invention may contain a pharmaceutically acceptable carrier. “Pharmaceutically acceptable carrier” allows use of the pharmaceutical composition in a method for the therapeutic treatment of humans or animals. In this particular case, the carrier can be a pharmaceutically suitable injectable carrier or diluent (for examples, see Remington's Pharmaceutical Sciences, 16^{th} ed. 1980, Mack Publishing Co). Such carrier or diluent is pharmaceutically acceptable, i.e. is non toxic to a recipient at the dosage and concentration employed. It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers or adjuvants. The pH of the pharmaceutical preparation is suitably adjusted and buffered in order to be useful in in vivo applications. It may be prepared either as a liquid solution or as a solid form (e.g.lyophilized) which can be suspended in a solution prior to administration. Representative examples of carriers or diluents for injectable formulation include water, isotonic saline solutions which are preferably buffered at a physiological pH (such as phosphate buffered saline or Tris buffered saline), mannitol, dextrose, glycerol and ethanol, as well as polypeptides or protein such as human serum albumin. For example, such formulations comprise the pharmaceutical composition prepared according to the use of the present invention in 10 mg/ml mannitol, 1 mg/ml HSA, 20 mM Tris pH 7.2 and 150 mM NaCl. Another preferred formulation comprises 1 M sucrose, 150 mM NaCl, 1 mM MgCl₂, 54 mg/l Tween 80, 10 mM Tris pH 8.5.

In accordance with these therapeutical aspects of the invention, a preferred embodiment relates to a method of delivery of the virus or virus-like particle of the invention, preferably, which contains a gene encoding a molecule having an indirectly cytotoxic function.

Suitably, the indirectly cytotoxic function refers to an enzyme that converts a prodrug to a toxic drug. With such a virus or virus-like particle, once the virus or virus-like particle has bound to the target cells, delivered its nucleic acid to the cells, and expressed the indirectly cytotoxic functions, which typically takes a day or so, the pro-drug is administered. The timing between administration of the virus or virus-like particle and the pro-drug may be optimised in a non-inventive way.

The dosage of the pro-drug will be chosen by the physician according to the usual criteria. The dosage of the virus or virus-like particle will similarly be chosen according to normal criteria, and in the case of tumour treatment, particularly with reference to the type, stage and location of tumour and the weight of the patient.

The duration of treatment will depend in part upon the rapidity and extent of any immune reaction to the virus or virus-like particle.

Some of the viruses or virus-like particles either in themselves, or together with an appropriate pro-drug, are in principle suitable for the destruction of cells in any tumour or other defined class of cells selectively exhibiting a recognisable (surface) entity. Examples of types of cancer that may be treated using the viruses or virus-like particles are those cited above, and especially cancers of the breast, prostate, colon, rectum, ovary, testicle and brain. The compounds are principally intended for human use but could be used for treating other mammals including dogs, cats, cattle, horses, pigs and sheep.

In a further preferred embodiment, the invention pertains to a pharmaceutical composition which comprises, in addition to the compounds mentioned above such as an adenoviral vector expressing a therapeutically useful gene as for example beta-interferon, at least one adjuvant capable of improving the transfection capacity or gene expression in the cell. Such an adjuvant can be selected from the group consisting of chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile or derivatives. The composition may also advantageously comprise a source of a cytokine which is incorporated in the form of a polypeptide or as a polynucleotide encoding the cytokine. Preferably, said cytokine is interleukin 10 (IL-10)(EP-A-967 289). The therapeutic composition can further comprise a nuclease inhibitor such as actine G, or specific magnesium or lithium concentrations.

Moreover according to a specific embodiment, the pharmaceutical composition comprises transformed a host cell which preferably may be a human muscular cell which is further encapsulated. Cell encapsulation methodology has been previously described which allows transplantation of encapsulated cells in treatment of Parkinson's disease (Tresco et al., 1992, ASAIO J., 38, 17-23) or amyotrophic lateral sclerosis (Aebischer et al., 1996, Hum. Gene Ther. , 7, 851-860). According to said specific embodiment, transformed cells are encapsulated by compounds which form a microporous membrane, and said encapsulated cells can further be implanted in vivo. Capsules, for example approximately 1 cm in length containing the cells of interest may be prepared employing a hollow microporous membrane fabricated from poly-ether-sulfone (PES) (Akzo Nobel Faser AG, Wuppertal, Germany; Déglon et al, 1996, Hum. Gene Ther., 7, 2135-2146). This membrane has a molecular weight cutoff greater than 1,000,000 Da, which permits the free passage of proteins and nutrients between the capsule interior and exterior, while preventing the contact of transplanted cells with host cells. The entrapped cells may be implanted by intradermal, subdermal, intravenous, intramuscular, intranasal, intracerebral, intratracheal, intraarterial, intraperitoneal, intravesical, intrapleural, intracoronary or intratumoral ways. In case where said transformed host cell is a myoblast, it can migrate from the site of injection to muscles where expression of the gene of interest (e.g. beta-interferon) can occur.

While the present invention has been described with reference to preferred embodiments and specific examples, one of the ordinary skill after reading the foregoing specification will be able to effect various changes, substitutions of equivalents, and other alterations to the processes and produced cells set forth herein. It is therefore intended that the protection claimed hereon be limited only by the definition contained in the appended claims and equivalents thereof.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nlm.nih.gov, http://www.infobiogen.fr, http://www.fni.ch/biology/research_tools.html, http://www.tigr.org, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced different from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### BRIEF DESCRIPTION OF THE DRAWINGS

FIGURE LEGENDS

FIG. 1 Conserved sequence elements in pIX. Aminoacid sequence alignments (Clustal X) of pIX from several human (top 8 sequences) and animal (bovine -b-, porcine -p- and canine -c-) Ad serotypes, as indicated on the left, were performed. Accession numbers are: Ad2 (p03282), Ad5 (p03285), Ad3 (J01962), Ad7 (03283); Ad9 (q9yl97); Ad12 (03484); Ad40 (p48312); Ad41 (p32539); Ad2b (q65377); Ad3p (q9w9x3); Ad1c (q65944); Ad2c (p14268). Dots correspond to gaps inserted by the program to optimise alignments. Conserved sequence elements are boxed. “*” and “ˆ ” (bottom) denote identical and related amino acid residues, respectively. Numbers in parentheses (top) refer to coordinates of amino acids in human Ad2 and Ad5 pIX, relative to the starting methionine.

FIG. 2 Schematic representation of specific subdomains of pIX. (A) The conserved pIX sequence domains, including the central human-specific poly-alanine stretch are represented as boxes, with relevant peptidic elements and coordinates from the Ad2 or Ad5 wt sequence. Point mutations or small deletions are indicated above, while larger deletions as mentioned in the text are depicted below. (B) The predicted helical-wheel representation of the C-terminal leucine-repeat of human Ad2 or Ad5 pIX, from residues 100 [first L at position (a)] to 134 [S at position (g)] is shown next to a symetrically positioned wheel of the same region. The potential hydrophobic interactions [(a)/(d′) and (d)/(a′)] between the 2 helices is suggested by the alignments of the residues at positions (a) and (d) with (d′) and (a′), respectively, in a putative coiled-coil structure. The charged residues (“+” or “−”) which presumably stabilise these interactions are indicated. Residues (with corresponding coordinates) that are altered by site-directed mutagenesis are bold-faced.

FIG. 3 The integrity of the conserved N-terminal domain of Ad2 pIX is required for capsidic incorporation. CsCl-purified Ad5 E1° virions expressing wt pIX, no pIX (E1°IX°) or specific pIX variants (as indicated on the top; “L-V” means L114P+V117D) were disrupted by boiling in SDS sample buffer, fractionated by 10%-SDS PAGE and analysed by immunoblotting using polyclonal anti-pIX antibody (even lanes named “v”). Extracts were prepared (44) from 293 cells that had been infected by the same viruses (MOI of 20 PFU per cell) and collected 36 h pi. Aliquots were analysed by immunoblotting (odd lanes named “e”), next to the corresponding virions. The expected position of pIX is indicated.

FIG. 4 The leucine-repeat and central hinge region are crucial for Ad2 pIX transcriptional activity. (A) Structure of the chimeric pE1a-CAT reporter plasmid in which the promoter region of the Ad5 E1 a transcription unit was fused to the CAT gene. (B) A549 cells were transfected with 1 μg of the pE1 a-CAT reporter plasmid, either alone (column 1) or together with plasmids expressing the wild type or mutated pIX as F-tagged fusion proteins: F/IX derivatives (0.1 μg; columns 2 and 3-14, respectively) or IX/F derivatives (0.5 μg; columns 15 and 16, respectively). Cells were collected 36 h later, and extracts were prepared. Relative CAT activities (means from 3 independent experiments) are represented with corresponding standard deviations.

FIG. 5 Self-interaction of pIX. A549 cells were transfected with vectors expressing the wt pIX, together with vectors expressing N-terminally F-tagged wt or mutated pIX proteins, as indicated. Cell extracts were prepared and aliquots were precipitated (IP) with monoclonal antibodies directed against the F epitope. The immunoprecipitates were submitted to Western-blot analysis (WB), probing first with the anti-F monoclonal antibody. After exposure, the same blot was washed and reprobed with polyclonal antibodies against pIX. The position of bands corresponding to the wt and F-tagged pIX are indicated. The bands marked by the asterisk likely correspond to proteolytic breakdown products of the F-tagged derivatives. The position of the immunoglobulin heavy subunit [IgG(H)] is indicated to show that equal amounts of antibody were used in the IP reaction.

FIG. 6 Sequence of the Adenoviral genome 5 as disclosed in the GenBank data bank under the reference M73260.

FIG. 7 illustrates that pIX protein actively induces specific nuclear inclusions. (A) Ad-2 infected A549 cells at intermediate stage of nuclear transformation (14-16h pi) were processed for immunogold labelling with anti-pIX polyclonal antibody on Lowicryl sections of formaldehyde-fixed cells ; fibrillo-granular network (fg), c.a. inclusions (star), viral single strand DNA (a), cytoplasm (c). Bar 0.5 μm. (B) Late stage of nuclear transformation (24-30 h pi) ; c.a. inclusions (star), electron-translucent region (e), perinuclear layer of host chromatin (ch), viral region (vr), virus (v), cytoplasm (c). Bar 0.5 μm. (C) Overexpression of recombinant pIX protein induces tha accumulation of the protein within newly formed c.a. inclusions; c.a. inclusions (star), perinuclear layer of condensed chromatin (ch), nucleolus (nu), Bar 0.5 μm.

### EXAMPLES

The examples which follow illustrate only one embodiment of the present invention.

The constructions described below are carried out according to the general techniques of genetic engineering and molecular cloning as detailed in Maniatis et al., (1989, Laboratory Manual, Cold Spring Harbor, Laboratory Press, Cold Spring Harbor, N.Y.).

The collective steps of cloning employing bacterial plasmids is carried out by passage in Escherichia coli (E. coli) strain 5K or BJ, whereas those employing vectors derived from phage M13 are carried out by passage in E. coli NM522. As regards the steps of PCR amplification, the protocol as described in PCR Protocols—A guide to methods and applications (1990, edited by Innis, Gelfand, Sninsky and White, Academic Press Inc.) is applied.

The fragments inserted into the different constructions described below are indicated precisely according to their position in the nucleotide sequence of:

the Ad5 genome, as disclosed in the GenBank data bank under the reference M73260,

the adenovirus type-2 (Ad2) genome, as disclosed in the GenBank data bank under the reference J01949, the SV40 virus genome, as disclosed in the GenBank data bank under the reference J02400.

The experiments described in the Examples can be summarized as follows: The product of adenovirus (Ad) type 5 gene IX (pIX) is known to actively participate in the stability of the viral icosahedron, acting as a capsidic cement. It has previously been demonstrated that pIX is also a transcriptional activator of several viral and cellular TATA-containing promoters, likely contributing to the transactivation of the Ad expression program. As it is described in the following, by extensive mutagenesis, the functional domains could be delinated that are involved in each of the pIX properties: residues 22 to 26 of the highly conserved N-terminal domain are crucial for capsidic incorporation of the protein; the C-terminal leucine-repeat is responsible for pIX interactions with itself and possibly other proteins, a property that is critical for pIX transcriptional activity. It could also be shown that pIX takes part in the viral-induced nuclear reorganization of late infected cells: through self-assembly, the protein induces the formation of specific nuclear structures which appear as dispersed nuclear globules by immunofluorescence staining and as clear amorphous spherical inclusions by electron microscopy. The integrity of the leucine-repeat is also essential for the formation of these inclusions. Together, the results demonstrate the multifunctional nature of pIX and provide new insights into Ad biology.

Introduction

Replication-deficient adenoviruses (Ad) efficiently transfer and express candidate therapeutic genes into a variety of dividing and post-mitotic cell types. For these reasons such viruses constitute effective vectors for direct in vivo gene therapy. However, several drawbacks such as toxicity, host inflammatory responses or transient in vivo transgene expression impair the full success of Ad vectors in human gene therapy protocols. Multiple factors are involved, among which some viral proteins whose functions are often not fully understood.

In the present invention, the attention focused on the the product of gene IX (pIX) from Ad serotypes 2 and 5 (Ad2 and Ad5). pIX is a small polypeptide of 140 residues (14.3 kDa), that is incorporated into the mature viral capsid. It is associated with hexon proteins to form group-of-nine hexons (GON) that make up the central region of each facet of the icosahedron. Precise determination of the stoichiometry of this assembly has revealed that there are 12 molecules of pIX, organized as four trimers per GON, and therefore 240 molecules per virion. The protein acts as a capsidic cement and thereby enhances the thermal stability of virions. It is essential for packaging 100% and more of the full length Ad DNA. By themselves, these properties of pIX appear important enough to be taken into consideration during the design of Ad vectors.

Additional observations strongly suggest that pIX is more than a capsidic protein and may serve additional functions during the infectious cycle: (i) gene IX is the only structural protein coding gene which is uncoupled from the Ad major late promoter (MLP); (ii) its expression pattern follows a different time course and begins at an intermediate time post-infection (pi), much earlier than that of all the other structural proteins; (iii) finally, pIX accumulates in the infected cell nuclei with a speckled distribution. In agreement with this nuclear localisation, it has previously been shown that pIX is a transcriptional activator of several viral and cellular TATA-containing promoters, among which the Ad E1a, E4 and MLP promoters. This finding led to the hypothesis that pIX could be involved in the transactivation of Ad genome expression.

To precisely delineate the finctional domains of pIX responsible for the structural and transcriptional properties, an extensive mutational analysis of the pIX coding sequence has been performed. The highly conserved N-terminal part of the protein could be shown to be essential for the capsidic structural properties, whereas the C-terminal leucine-repeat (putative coiled-coil domain) is critical for the transactivating function. Accumulation of pIx results in the formation of specific nuclear structures (the clear amorphous inclusions), the function of which is presently unknown. The present results suggest that formation of these structures involves self-assembly of pIX through its coiled-coil domain.

Materials and Methods

Cells and viruses

Monolayer human A549 cells were grown in Dulbecco's medium supplemented with 10% fetal calf serum (FCS). 293 cells were grown in Dulbecco's modified Eagle medium with 2% FCS. A549 cells (at 80% confluence) were infected with wild type (wt) Ad2 or Ad5 at a multiplicity of infection (MOI) of 50 plaque forming units (PFU) per cell. Mutant viral genomes were constructed as infectious plasmids by homologous recombination in Escherichia coli, as described. [Chartier et al., 1996, J. Virol. 70, 4805-4810] All vectors contain, in addition to alterations of gene IX (see below), a deletion in E1 (between nucleotides 459 and 3331) and in E3 (between nucleotides 28592 and 30470) (Ad E1° E3°). Nucleotide numbering throughout this paper conforms to that of Chroboczek et al. Mutant viruses were amplified in 293 cells. Viral growth, titration, and storage were previously described. [Lusky et al., J. Virol. 72, 2022-2032]

Recombinant Eukaryotic Expression Vectors

The sequence encoding wild type (wt) pIX was derived from the Ad5 genome by PCR amplification as previously described [Lutz et al., 1997, J. Virol. 71, 5102-5109], and inserted into 3 types of expression vectors: (i) the pAT4 vector (gift from M. Vigneron), in a site located 3′ to the sequences encoding the F domain of the human estrogen receptor (hER), generating a wt pIX fusion protein tagged at its N-terminus (F/IX); (ii) the pXJ41 vector, which yields an untagged protein; (iii) the pXX vector, a pSG5-derived vector, where the sequence encoding wt pIX was inserted in a site located 5′ to the sequences encoding the F domain of the human estrogen receptor, generating a pIX fusion protein tagged at its C-terminus (IX/F). In these vectors, the expression of the wt or mutated pIX sequences was directed by the cytomegalovirus (CMV) enhancer and herpes simplex virus type-1 (HSV-1) thymidine kinase gene promoter (pAT4 and pXJ41), or the SV40 promoter (pSG5-derivative pXX).

The Ad E1a promoter sequence positions +100 to +560, numbering to Chroboczek et al.) was subcloned in front of the chloramphenicol acetyl transferase (CAT) reporter gene of the promoterless pBLCAT6 vector, as previously described. [Lutz et al., 1997, J. Virol. 71, 5102-5109

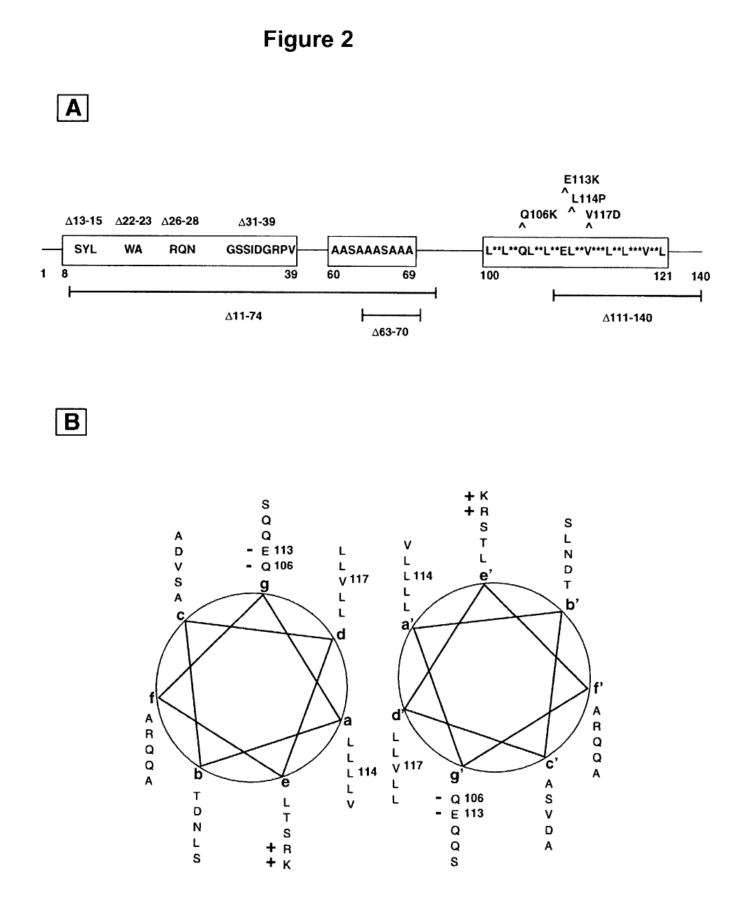

Point mutations and deletions in the pIX coding sequence (as indicated in FIG. 2A) were generated by following the protocol of the “QuickChange site-directed mutagenesis” system (Stratagene catalog#200518). All plasmids were verified by sequencing.

Oligonucleotides sequences

(SEQ ID NO:2)

| | |
|---|---|
| Deletion WA: | 5′ GACAACGCGCATGCCCCCA*GGGGTGCGTCAGAATGTG 3′ |
| and | |

(SEQ ID NO:3)

3′ CTGTTGCGCGTACGGGGGT*CCCCACGCAGTCTTACAC 5′

* is deletion of 6 bases 5′ TGGGCC 3′ (3672-3677, with reference to Ad5)
and 3′ ACCCGG 5′

(SEQ ID NO:4)

| | |
|---|---|
| Deletion SYL: | 5′ GATGGAAGCATTGTGAGC*ACAACGCGCATGCCCCCATGGGCCGGGG 3′ |
| and | |

(SEQ ID NO:5)

3′ CTACCTTCGTAACACTCG*TGTTGCGCGTACGGGGGTACCCGGCCCC 5′

* is deletion of 9 bases 5′ TCATATTTG 3′ (3645-3653) and 3′ AGTATAAAC 5′

(SEQ ID NO:6)

| | |
|---|---|
| mutation RQN: | 5′ CCCCCATGGGCCGGGGTGCTCGACGTGATGGGCTCCAGC 3′ |
| and | |

(SEQ ID NO:7)

3′ GGGGGTACCCGGCCCCACGAGCTGGTGATGGGCTCCAGC 5′

(SEQ ID NO:8)

| | |
|---|---|
| mutation GSSIDGR: | 5′ GGGCCGGGGTGCGTCAGAATGTGATGCATATGCCCGTCCTGCCCGC 3′ |
| and | |

(SEQ ID NO:9)

3′ CCCGGCCCCACGCAGTCTTACACTACGTATACGGGCAGGACGGGCG 5′

(SEQ ID NO:10)

| | |
|---|---|
| mutation L114P: | 5′ GGATTCTTTGACCCGGGAGCCCAATGTCGTTTCTCAGC 3′ |
| and | |

(SEQ ID NO:11)

3′ CCTAAGAAACTGGGCCTCGGGTTACAGCAAAGAGTCG 5′

(SEQ ID NO:12)

| | |
|---|---|
| mutation V117D: | 5′ GGGAACTTAATGTCGACTCTCAGCAGCTGTTGG 3′ |
| and | |

(SEQ ID NO:13)

3′ CCCTTGAATTACAGCTGAGAGTCGTCGACAACC 5′

(SEQ ID NO:14)

| | |
|---|---|
| mutation L114P/V117D: | 5′ GGATTCTTTGACCCGGGAGCCCAATGTCGACTCTCAGCAGCTGTTGG 3′ |
| and | |

(SEQ ID NO:15)

3′ CCTAAGAAACTGGGCCTCGGGTTACAGCTGAGAGTCGTCGACAACC 5′

(SEQ ID NO:16)

| | |
|---|---|
| mutation poly ala: | 5′ CGCCGTTGGAGACTCCATGGACCGCCCGCGGG 3′ |
| and | |

(SEQ ID NO:17)

3′ GCGGCAACCTCTGAGGTACCTGGCGGGCGCCC 5′

(SEQ ID NO:18)

| | |
|---|---|
| mutation E113K: | 5′ GGATTCTTTGACCCGGAAACTTAATGTCGTTTCTCAGC 3′ |
| and | |

(SEQ ID NO:19)

3′ CCTAAGAAACTGGGCCTTTGAATTACAGCAAAGAGTCG 5′

(SEQ ID NO:20)

| | |
|---|---|
| mutation Q1016K: | 5′ GACGGCTCTTTTGGCAAAGCTTGATTCTTTGACCCGGG 3′ |
| and | |

(SEQ ID NO:21)

3′ CTGCCGAGAAAACCGTTTCGTTCTAAGAAACTGGGCCC 3′

Transfections, Cell Extracts and Western-blotting

A549 cells were transfected by calcium phosphate coprecipitation. For CAT assays, the cells were harvested 36 h after transfection, extracts were prepared and aliquots, normalised by protein concentration, were assayed for CAT activity as described. [Bocco et al., 1993, Oncogene 8, 2977-2986] CAT activities were determined from at least 3 independent experiments and quantitated with a Bioimaging analyzer (Fuji Photo Film Co.).

For immunoprecipitations, the cells were harvested 36 h after transfection, by 3 cycles of freeze-thaw in buffer A (50 mM Tris-HCl, pH 7.9; 20% glycerol; 1 mM DTT; and 0.1% NP40) containing 0.4 M KCl. The expression of recombinant proteins was verified by Western blotting. After an additional clearing step on protein G-sepharose to adsorb non-specific binding proteins, cell extracts were incubated for 2 h with 1 μg of the anti-F antibody, after which 30 μl of protein G-sepharose beads were added and incubation was continued for an additional 2 h. The beads were then washed 3 times at room temperature with buffer A containing 200 mM KCl and 0.5% NP40 (mild-salt conditions). The resin was then dissociated by boiling for 5-10 min in SDS sample buffer. The bound proteins were detected on Western blots with specific antibodies using the ECL system (Amersham), as previously described. [Bocco et al., 1993, Oncogene 8, 2977-2986] Anti-pIX rabbit polyclonal antibodies were raised against purified recombinant GST-IX fusion protein (anti-pIX). Monoclonal antibodies against the F domain of the hER (Mab3A6) have been described. [Lutz et al., 1997, J. Virol. 71, 5102-5109]

Electron Microscopy

A549 cells near confluence were infected at a MOI of 5-10 PFUs of Ad5 per cell for 30 min. Then, monolayers were rinsed with PBS, fresh medium was added and the cells were reincubated for 16 and 30 h before fixation. A549 cells were transfected with the vector generating the untagged wt pIX and cultured for 36 h. The cells were fixed with 4% formaldehyde (Merck) in 0.1 M Sorensen's phosphate buffer, pH 7.2, at 4-8° C., for 1 h. During the fixation step, the cells were scraped from their plastic substrate and centrifuged. The resulting pellets were dehydrated in increasing concentrations of methanol and embedded in Lowicryl K4M (Polysciences Europe GmbH, Germany). Polymerisation was performed at-30° C., for 5 days, under long wavelength UV light (Philips fluorescence tubes TL 6W) and subsequently at room temperature for 1 day. Ultrathin sections were collected on Formvar-carbon coated copper grids, mesh 200.

For identifying structures containing the pIX viral protein, grids bearing Lowicryl sections were floated for 2 min over drops of Aurion BSA-C (purchased from Biovalley, France) (0.01% in PBS) in order to prevent background, prior incubation (30 min at room temperature) in the presence of anti-pIX polyclonal antibody diluted 1/50 in PBS. After rapid washing in PBS, grids were incubated at room temperature, for 30 min in the presence of goat anti-rabbit IgG conjugated to gold particles, 10 nm in diameter (British Biocell International LTD, Cardiff, UK) and stained with uranyl acetate prior to observation with a Philips 400 transmission electron microscope, at 80 kV and 6 000 to 22 000-fold magnification. To make sure that secondary antibody did not bind non-specifically to biological material, it was verified that no labelling occurred when primary antibody was omitted.

Immunofluorescence

Immunofluorescence staining experiments were carried out as previously described. [Lutz et al., 1997, J. Virol. 71, 5102-5109] A549 cells were fixed with formaldehyde (2% vol/vol in PBS) and permeabilised with 0.1% Triton X-100 in PBS. The primary antibodies were diluted in PBS containing 0.1% Triton X-100. The anti-pIX rabbit polyclonal antibody was used as described [Lutz et al., 1997, J. Virol. 71, 5102-5109] and the Mab3A6 anti-F antibody was used at 1/5000 dilution in PBS containing 0.1% Triton X-100. After incubation for 1 h, the coverslips were washed several times in PBS-0.1% Triton X-100 and then incubated with goat Texas-Red-conjugated anti-rabbit IgG and/or donkey FITC-labelled anti-mouse IgG (Sigma), at concentrations recommended by the suppliers. Nuclei were counter-stained with Hoechst 33258. After staining, the coverslips were mounted and analysed using a confocal laser scanning microscope (Leica). Image enhancement software was used to balance signal strength and 8-fold scanning was used to separate signal from noise.

### Example 1

### Peptide Sequence and Functional Domains of pIX

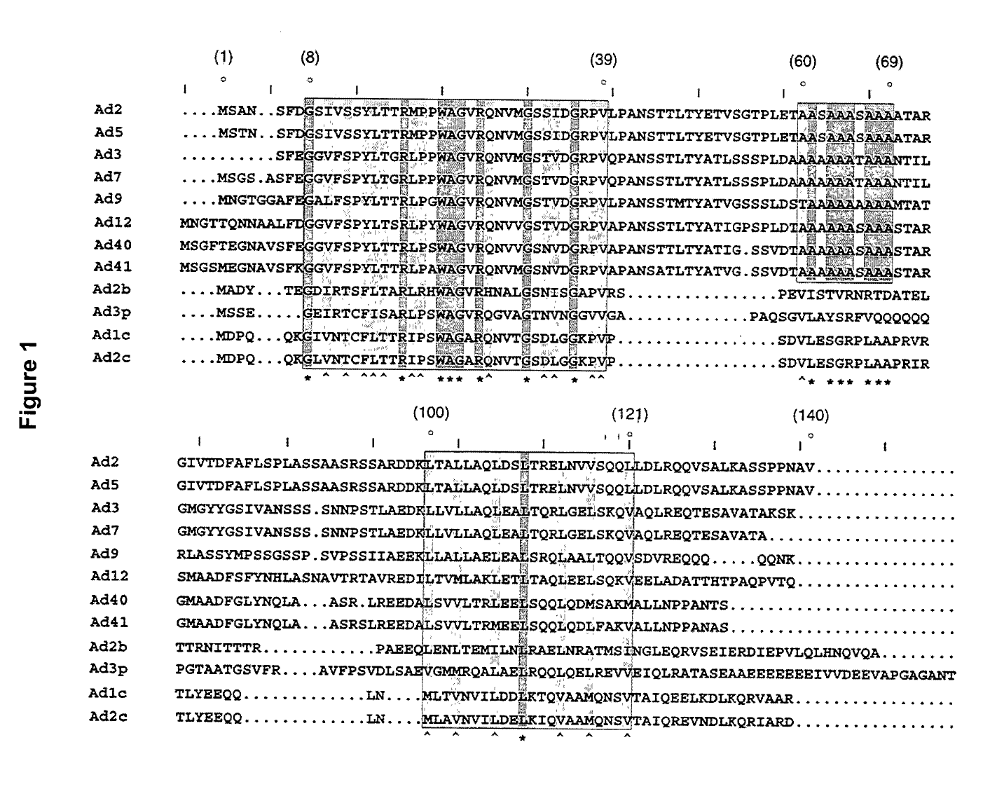

Multiple sequence alignments between pIX proteins from human and animal Ad serotypes (FIG. 1) revealed a high degree of identity (95%) over the entire length of pIX from serotypes belonging to the same subgenus. Although the extent of homology between serotypes from different species was lower, two conserved domains could be identified when comparing human and animal serotypes: referring to the coordinates of pIX residues (Aa) from the human Ad2 serotype, these domains locate at the N-terminal (Aa8-39) and C-terminal (Aa100-121) ends of pIX, respectively. An additional, alanine-rich domain, specific to the human serotypes, could be delineated (Aa60-69).

No particular structural motif could be identified within the N-terminal domain. The alanine-rich stretch which is unlikely to adopt any particular structure and may serve as a flexible link between the two halves of the pIX molecule. By contrast, the C-terminal domain clearly revealed features of a leucine-repeat (or coiled-coil domain), as suggested by the helical wheel representation (FIG. 2B): ten non polar amino acid residues (leucine and valine), spaced every 3 and 4 residues, align at positions (a) and (d) on one side of the wheel; these residues presumably provide a hydrophobic interface to interact with similar residues, symmetrically positioned (a′) and (d′) on a second monomer (FIG. 2B), thereby potentially adopting a coiled-coil conformation (34, 35, 39, 40). Moreover, in the case of Ad2 and Ad5, ionised residues of opposite charge, located on either side of the helical wheel at positions (e) and (g), may further stabilise protein assembly by symmetrically interacting with corresponding residues at positions (e′) and (g′).

To determine the functional significance of these domains, the effects of a series of deletions or point mutations altering the conserved sequence elements were examined (FIG. 2A). Within the N-terminal half of pIX, amino acid stretches that are most highly conserved between all (Aa13-15, Aa22-23, Aa26-28, Aa3l-39) or only human Ad serotypes (Aa63-70) were deleted (delta). The C-terminal leucine-repeat was disrupted by changing, either separately or simultaneously, leucine 114 and valine 117 to proline and aspartate, respectively, to generate mutants L114P, V117D or L114P-V117D (L-V). Interruption of the apolar series at positions (a) and (d) by these residues were indeed expected to disturb the correct alignment (proline) or hydrophobic bonding (aspartate). Two mutated forms of pIx were also constructed in which the net charge at position (e) was inverted by exchanging Aa106 or Aa113 with a lysine residue (mutants Q106K and E113K, respectively), thereby triggering electrostatic repulsion between protein monomers.

### Example 2

### The N-terminal Part of pIX Critically Contributes to its Capsidic Incorporation

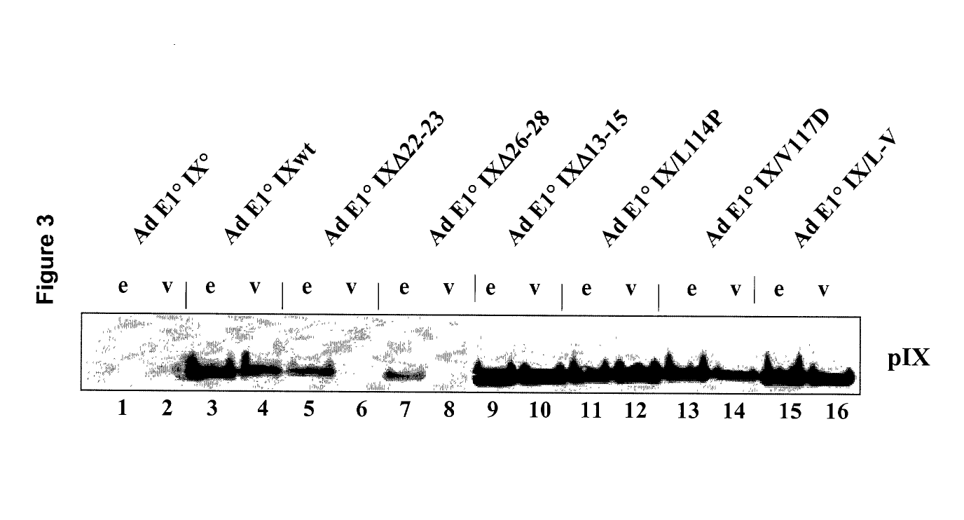

Earlier immunoelectron microscopy studies with purified Ad virions and pIX-specific antisera revealed that only the C-terminal portion of pIX was accessible to the antibodies. The authors concluded that the C-terminal part of the protein was exposed at the surface of the virions, while its N-terminal domain was hidden inside the viral capsid. To further define the pIX elements involved in the capsidic assembly, the mutations described above were introduced into the E1-deleted viral genome by homologous recombination to generate viruses expressing the pIX variants during viral production. Because all viruses are E1-defective, they were produced on the 293 cells which constituvely express the E1a and E1b Ad5 genes. Each pIX mutant virus was grown on 293 cells and viral particles were easily purified by density gradient centrifugation. After titration, 2.10¹⁰ particules virions (v) were submitted to SDS-PAGE, in parallel with aliquots of the corresponding infected crude extracts (e) (FIG. 3). The presence of pIX was then examined by Western-blot analysis, using anti-pIX polyclonal antibodies. As a positive control, the presence of the wt pIX protein was verified both in infected-cell extracts and purified Ad E1° virions (FIG. 3, lanes 3 and 4), whereas it was absent in both fractions from cells infected with a Ad E1° lacking gene IX (FIG. 3, lanes 1 and 2).

Mutations altering pIX within its C-terminal part (L114P, V117D and L-V) did not prevent incorporation of the mutant protein into the capsid (FIG. 3, lanes 12, 14 and 16), indicating that the integrity of the leucine-repeat is not required for this function. Moreover, capsid stability appeared not to be altered (data not shown). By contrast, deletions within the conserved N-terminal domain of pIX, like in mutants delta22-23 and delta 26-28, completely abolished incorporation of pIX into the capsid (FIG. 3, lanes 6 and 8), despite nearly normal levels of pIX synthesis (FIG. 3 lanes 5 and 7). Mutation (delta 13-15) did not impair virion insertion of pIX but the resulting capsid was less stable, as indicated by thermolability measurements (data not shown).

Together, these results define the N-terminal region spanning Aa22-28 as crucial for the correct and stable recruitment of pIX into the viral capsid, whereas the central (data not shown) and C-terminal regions are not involved at all.

### Example 3

### The Integrity of the C-terminal Leucine-repeat and Central Alanine-stretch of pIX are Essential for its Transcriptional Activity

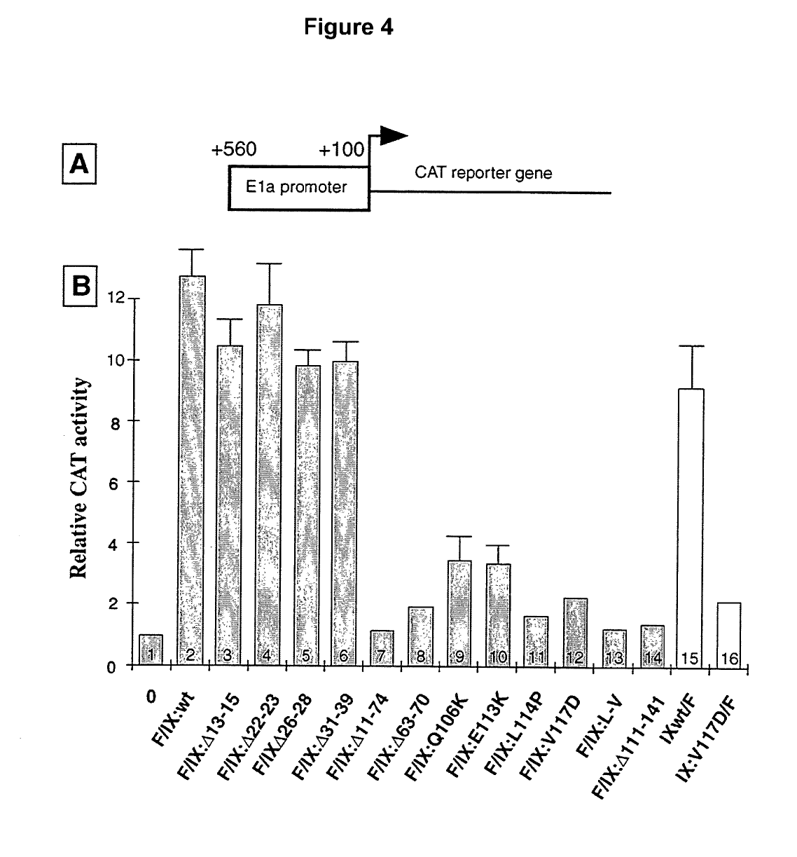

It has previously been shown that pIX exhibits transcriptional properties. [Lutz et al., 1997, J. Virol. 71, 5102-5109] Recombinant pIX efficiently stimulated, in a dose-dependent manner, the activity of several viral and cellular TATA-containing promoters. To precisely delineate the transactivating domain of pIX, the effect of the complete set of pIX mutations on E1a promoter activation was examined (FIG. 4). To this end, vectors expressing wild type or mutated piX sequences as proteins fused to the F epitope tag (F/IX) were transfected together with a CAT reporter gene driven by the E1a promoter. After having verified that equal levels of pIX were expressed, as revealed by immunoblotting with antibodies against the F epitope (data not shown), CAT activities were measured. Under these conditions, relative CAT activities will reflect the intrinsic transcriptional activating capacity of each recombinant protein.

In agreement with the earlier structural analysis, truncated versions of pIX lacking half of the leucine-repeat (F/Ixdelta 111-140) or most of the N-terminal half (F/IXdelta11-74) of the protein, lost their transactivating property (compare in FIG. 4B, columns 2 and 14 or 7 respectively)

Point mutations within the sequence encoding the leucine-repeat in the C-terminal end of pIX severely reduced reporter stimulation: single (L1 4P, V1 7D) or double alterations (L-V) had effects similar to complete deletion (deltalll-140) of the C-terminal part of the protein (FIG. 4B, compare column 2 with 11-14). Point mutations Q106K and E113K also reduced transactivation (FIG. 4B, compare columns 2 and 9-10), stressing the contribution of electrostatic interactions in functional assembly of pIX monomers. The transactivation function of pIX also depends on the integrity of the central domain, since deletion of the corresponding poly-alanine stretch (delta63-70) led to a strong reduction of reporter stimulation (FIG. 4B, compare columns 2 and 8). By contrast, deletions within the N-terminal part of pIX had no detectable effect on its intrinsic stimulatory activity, since very similar levels of transactivation were obtained with F/IX: delta 13-15, delta 22-23, delta 26-28, delta 31-39 pIX variants and the wild type protein (FIG. 4B, compare columns 2 and 3-6).

Together, the results described herein suggest that the pIX transactivating function is dependent on the integrity of the C-terminal leucine-repeat, as well as on the central alanine-rich element. Interestingly, the N-terminal region, critically involved in capsidic integration of pIX, is not involved in this function.

### Example 4

### The integrity of the C-terminal Leucine-repeat and Central Alanine-stretch of pIX are Essential for its Self-interaction

The presence of a leucine-repeat type of structure at the C-terminal end of pIX suggests that the protein may dimerise (or multimerise) by interacting through this element. To test this possibility, a vector expressing the non-tagged wt pIX was co-transfected into A549 cells with vectors expressing F epitope-tagged wild type or mutant pIX proteins (F/IX). As revealed by Western-blot analysis of cell extracts with monoclonal anti-F or polyclonal anti-pIX antibodies, the transfected vectors were expressed to very similar levels (data not shown).

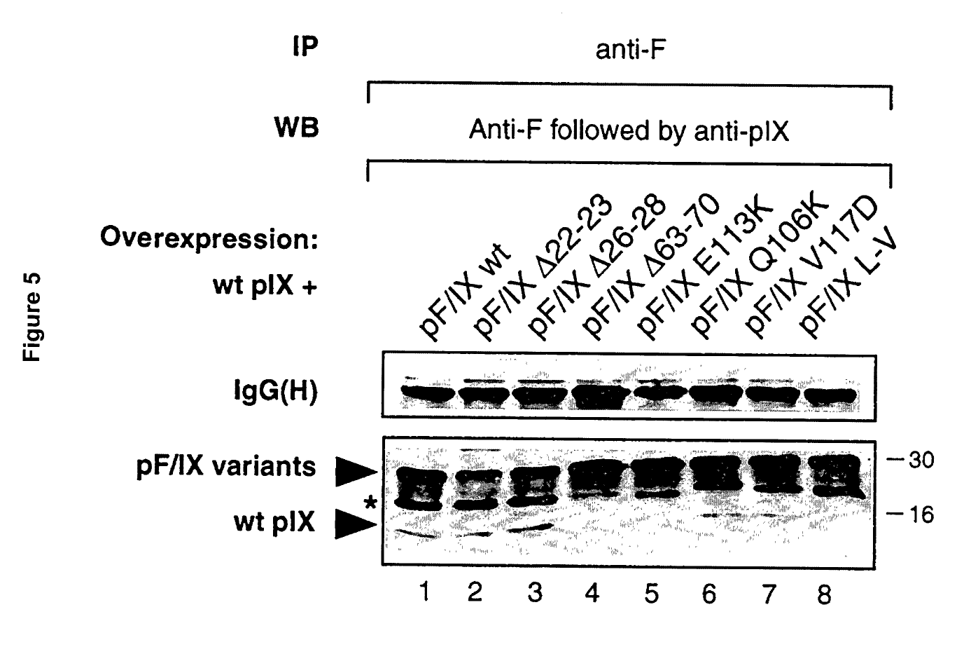

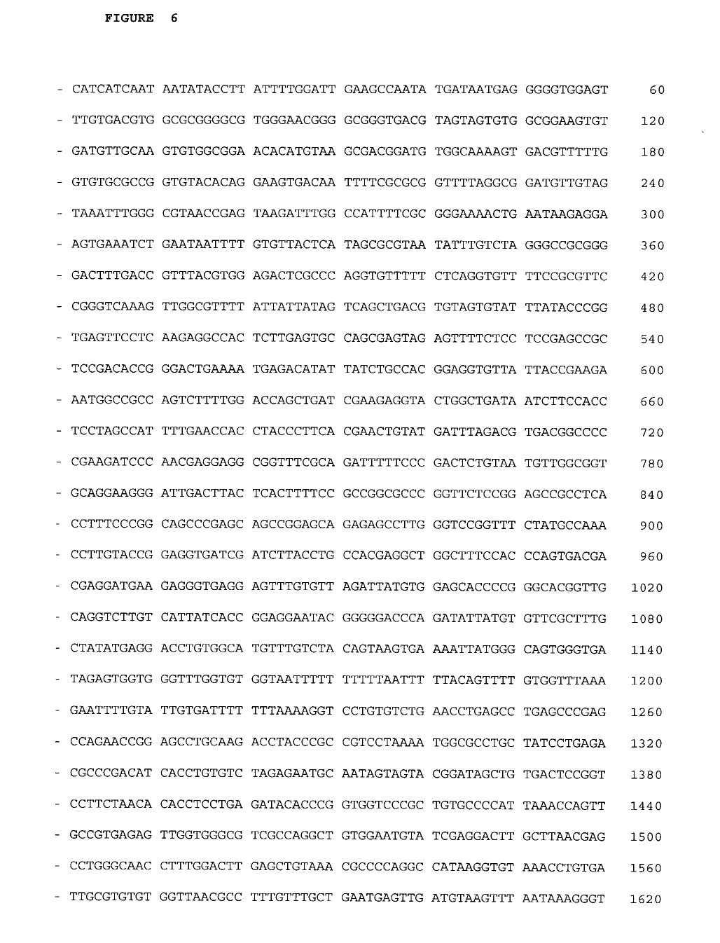

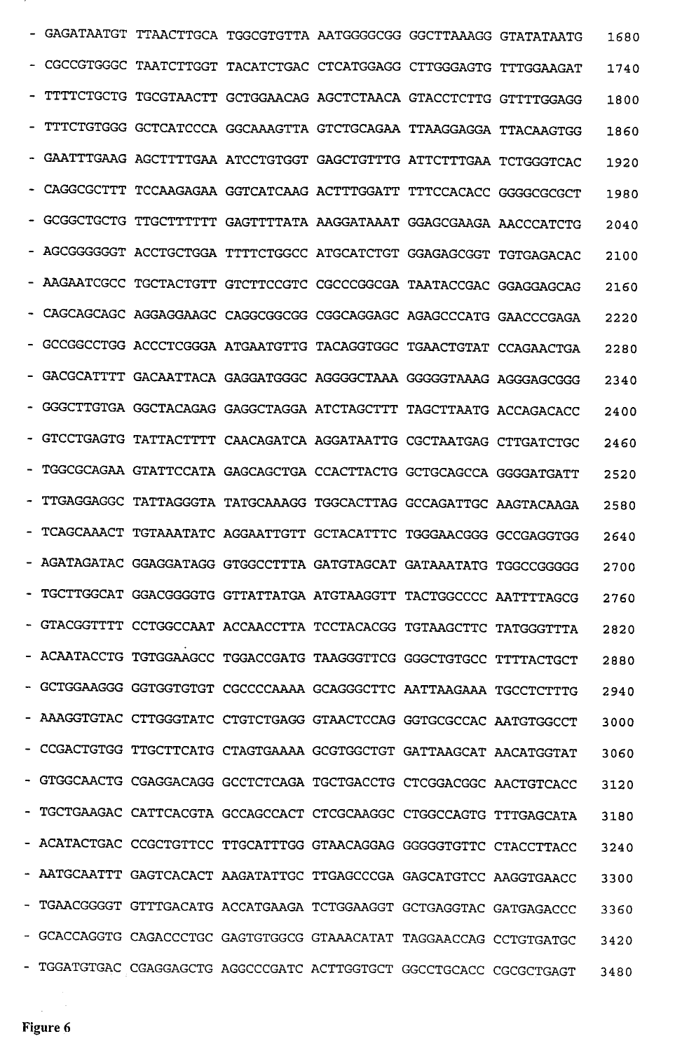

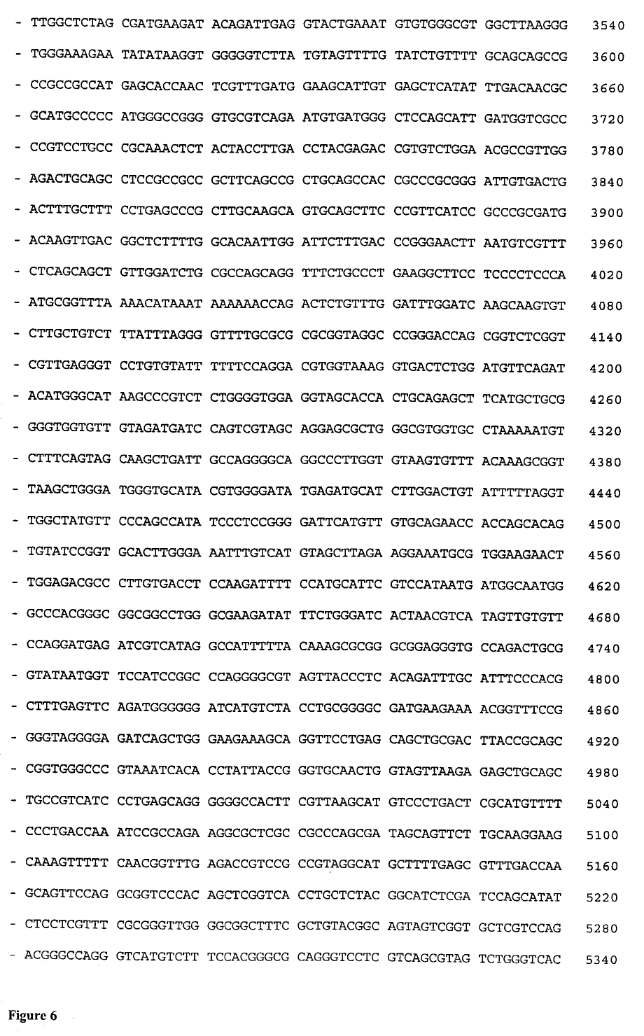

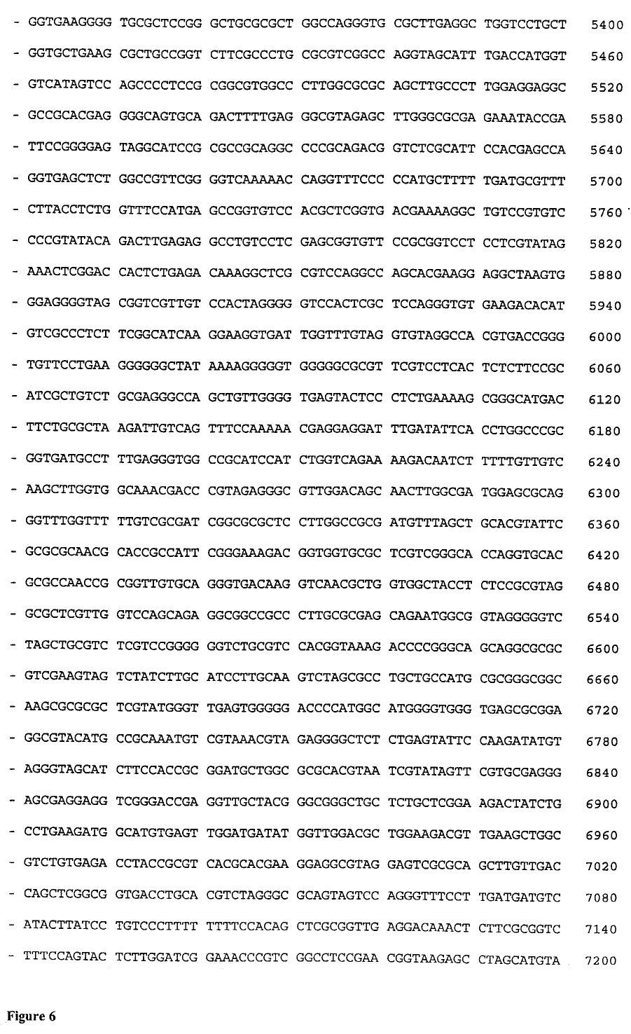

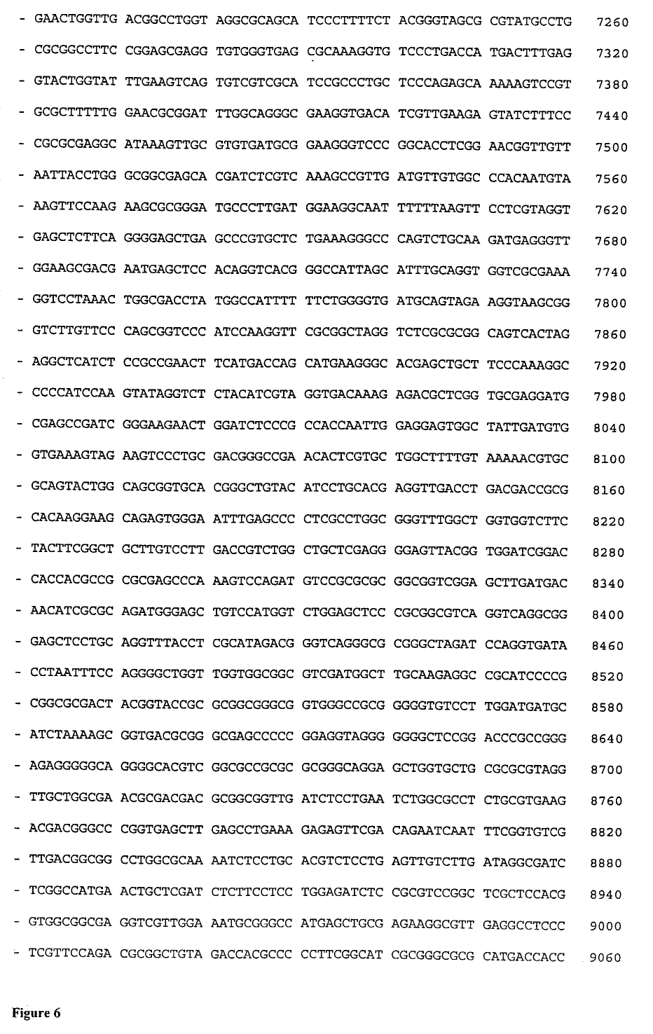

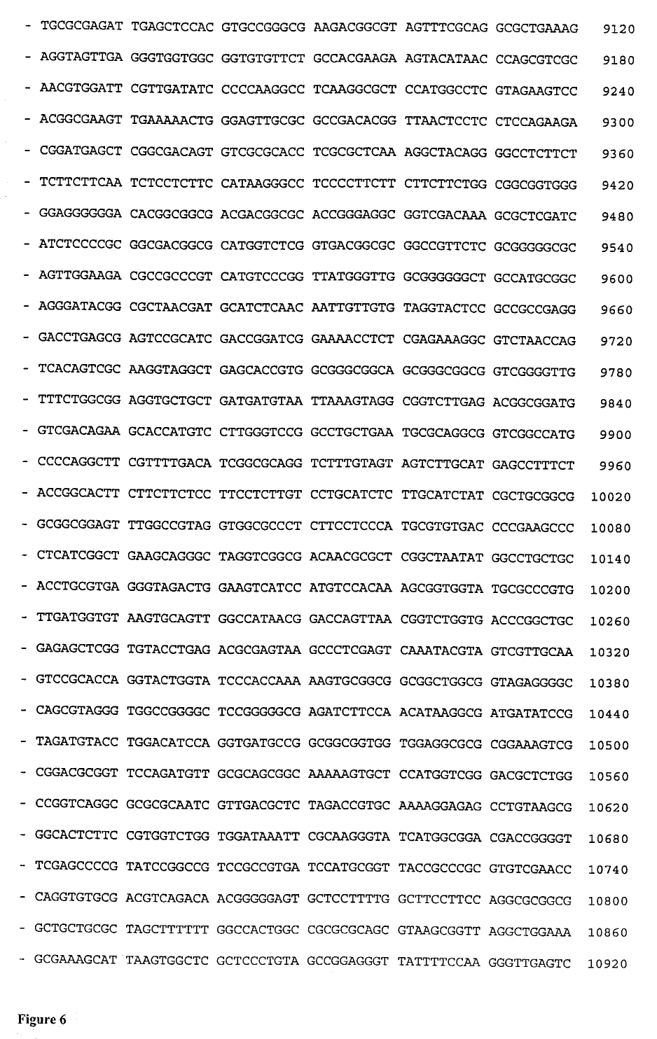

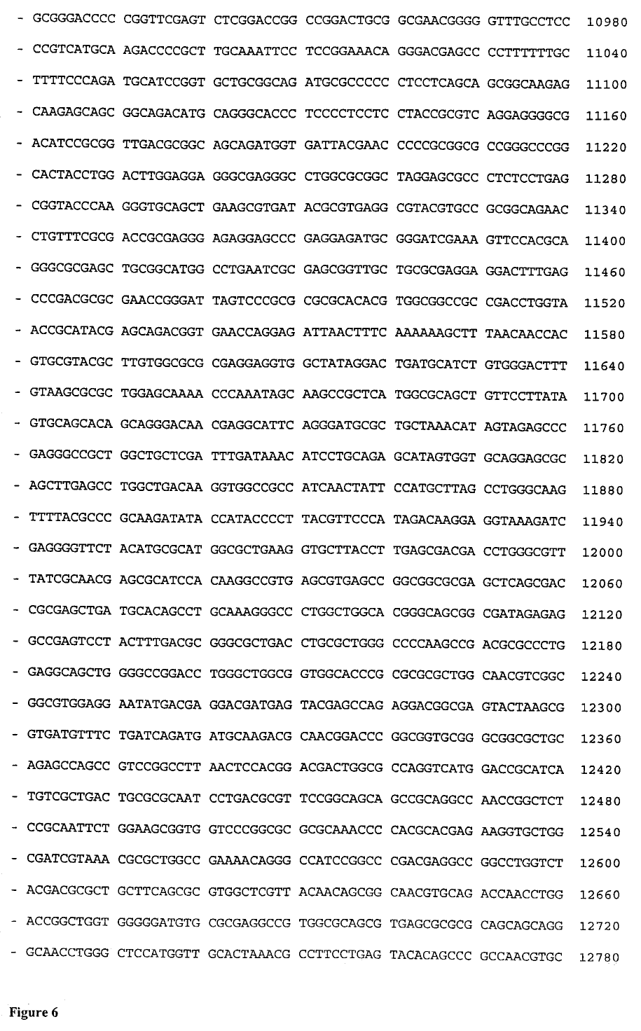

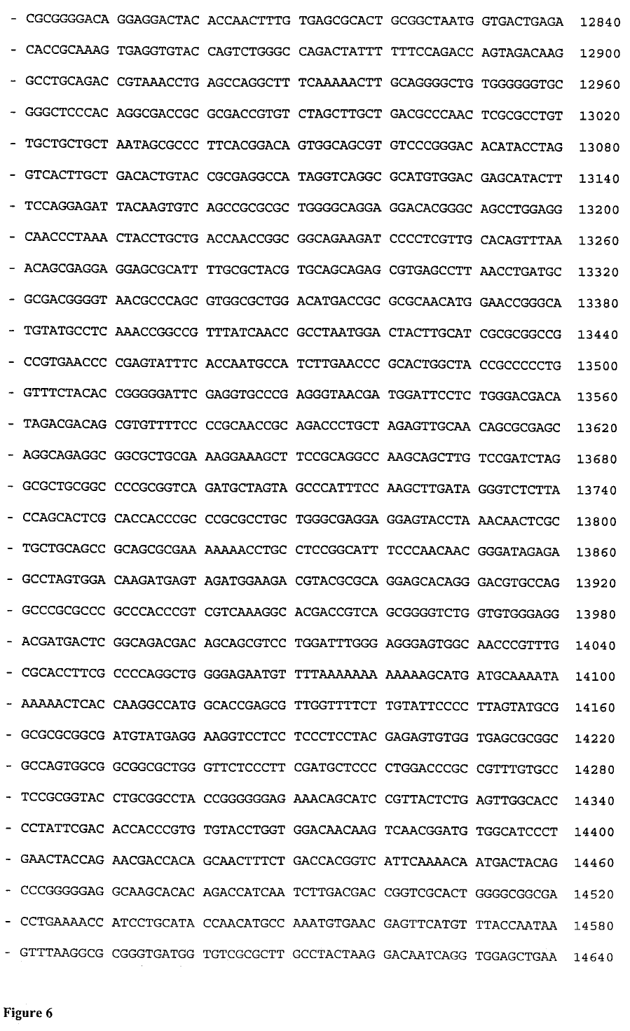

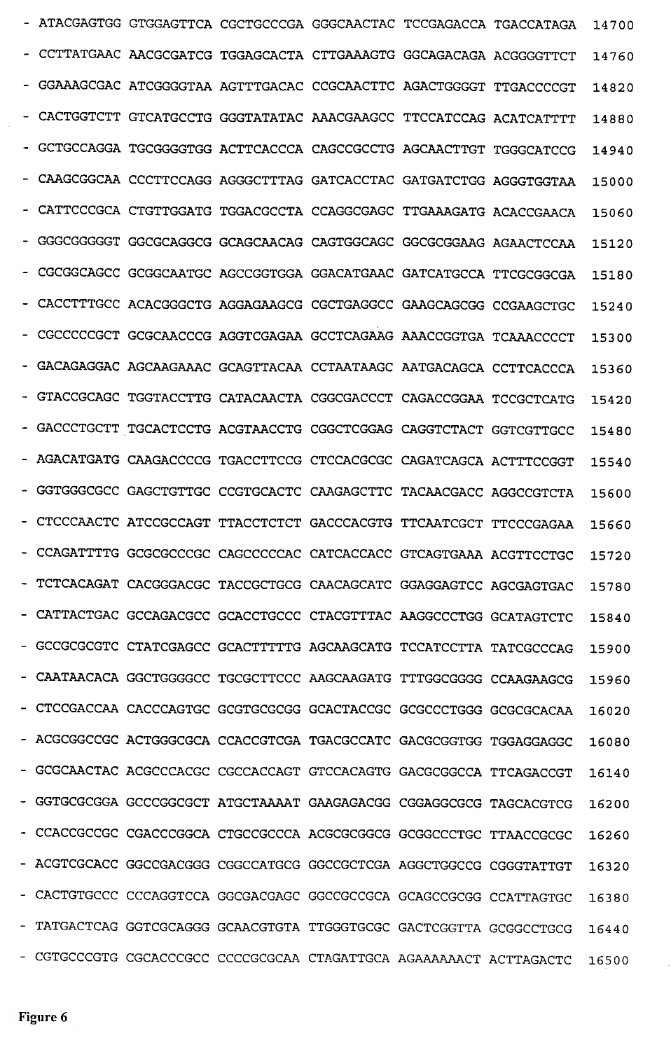

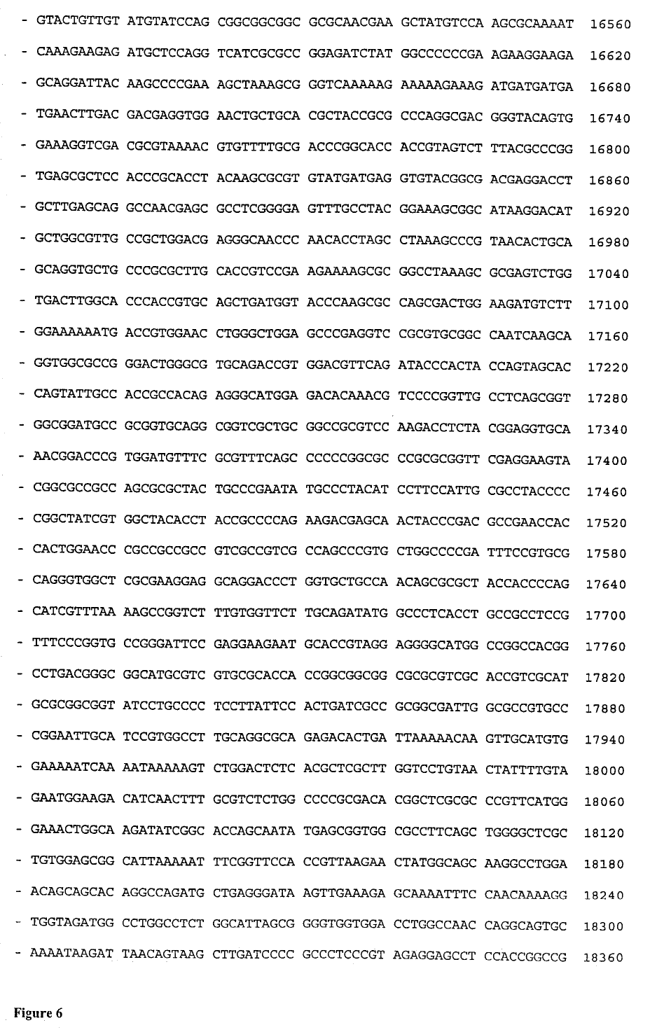

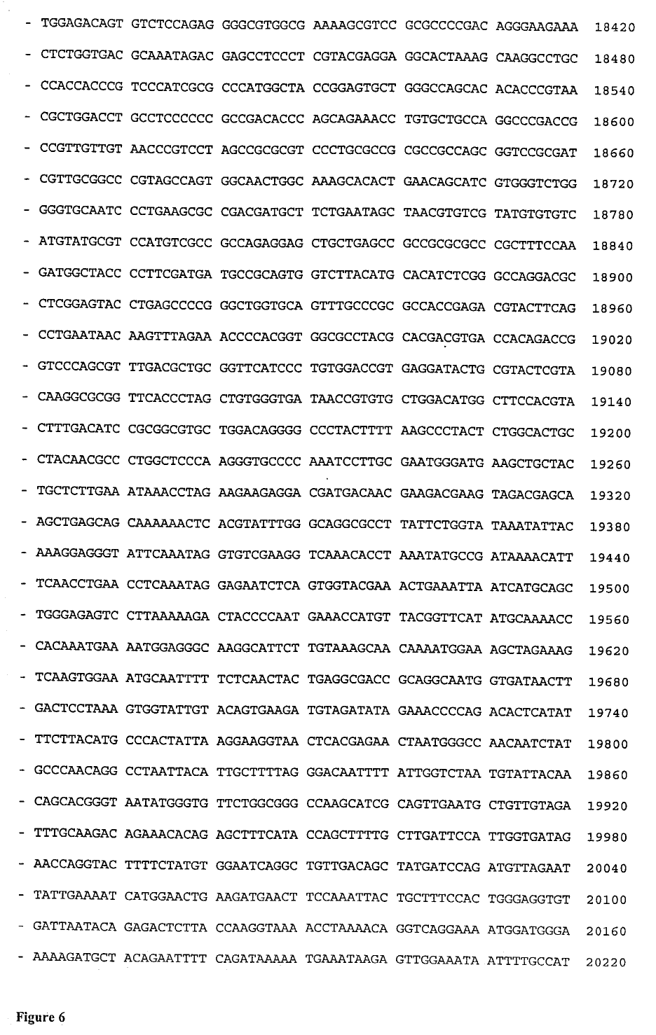

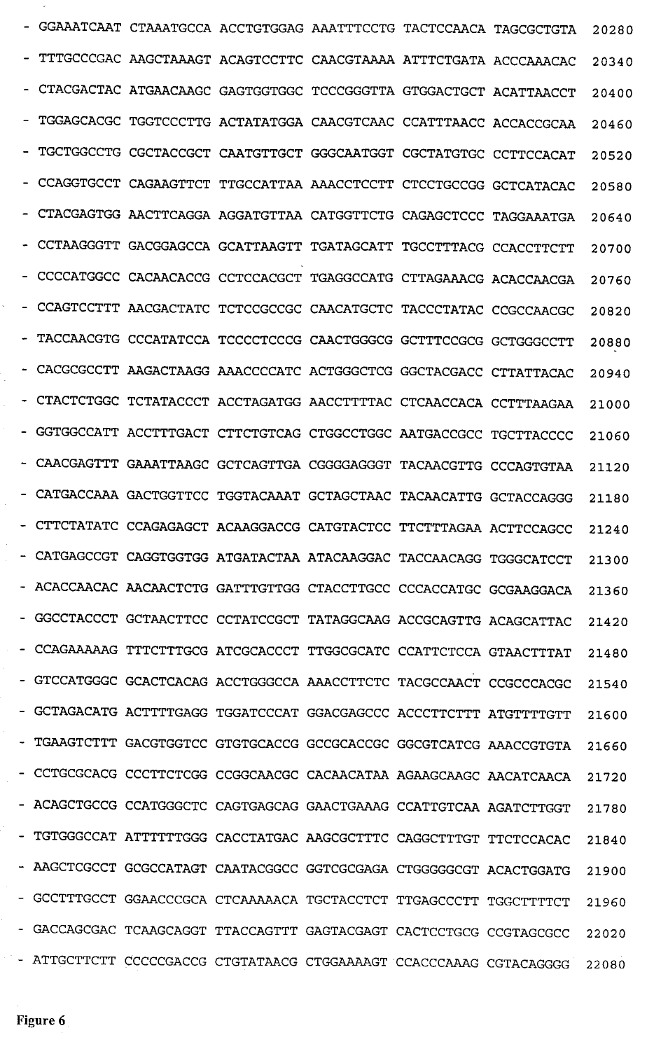

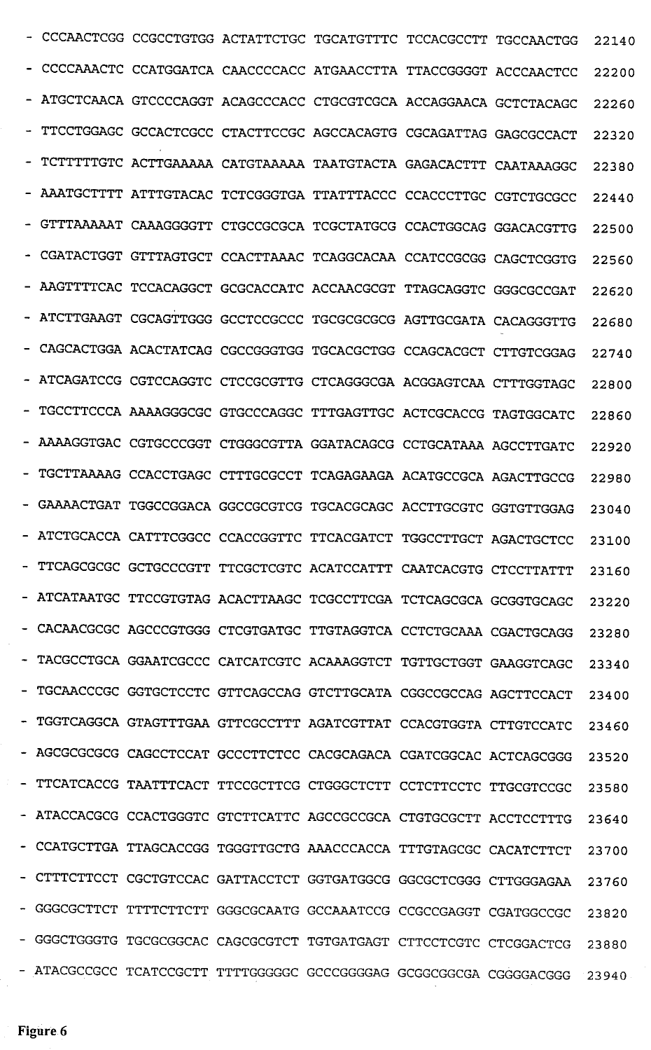

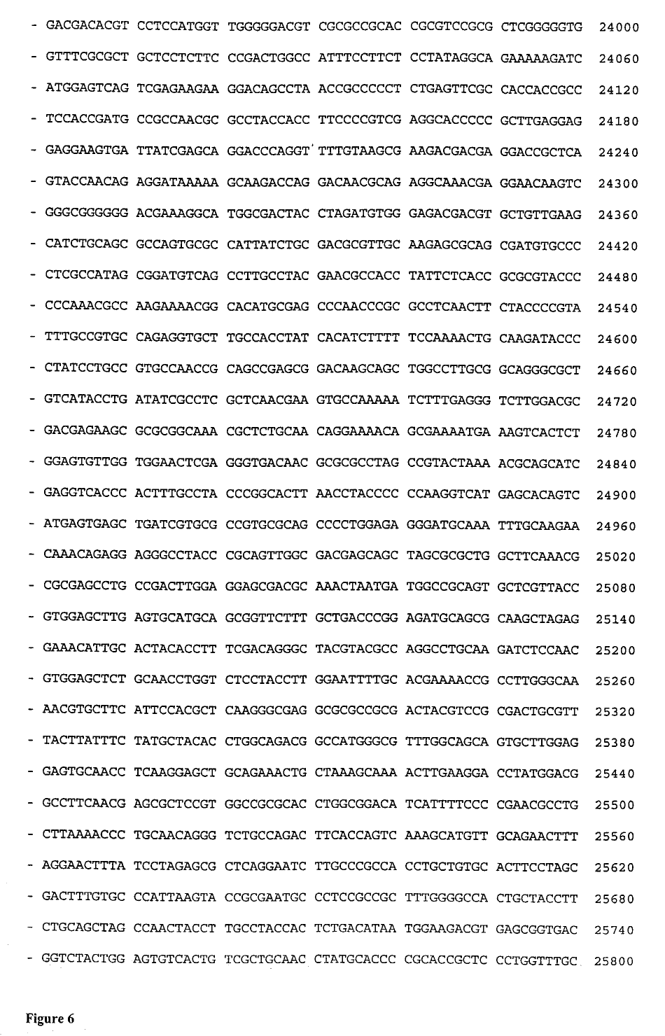

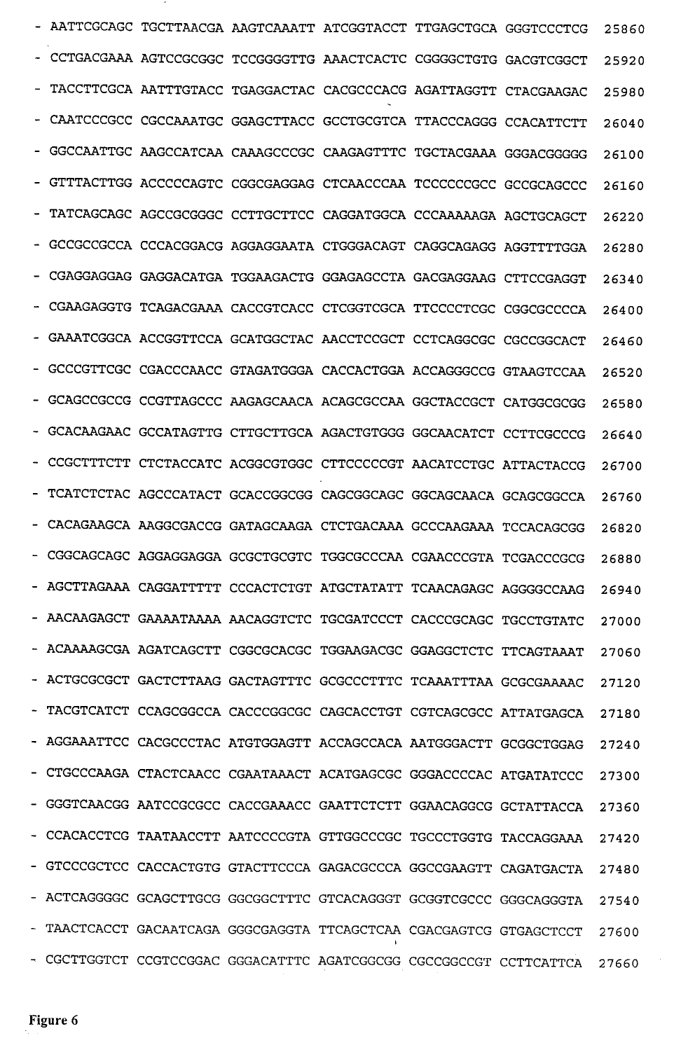

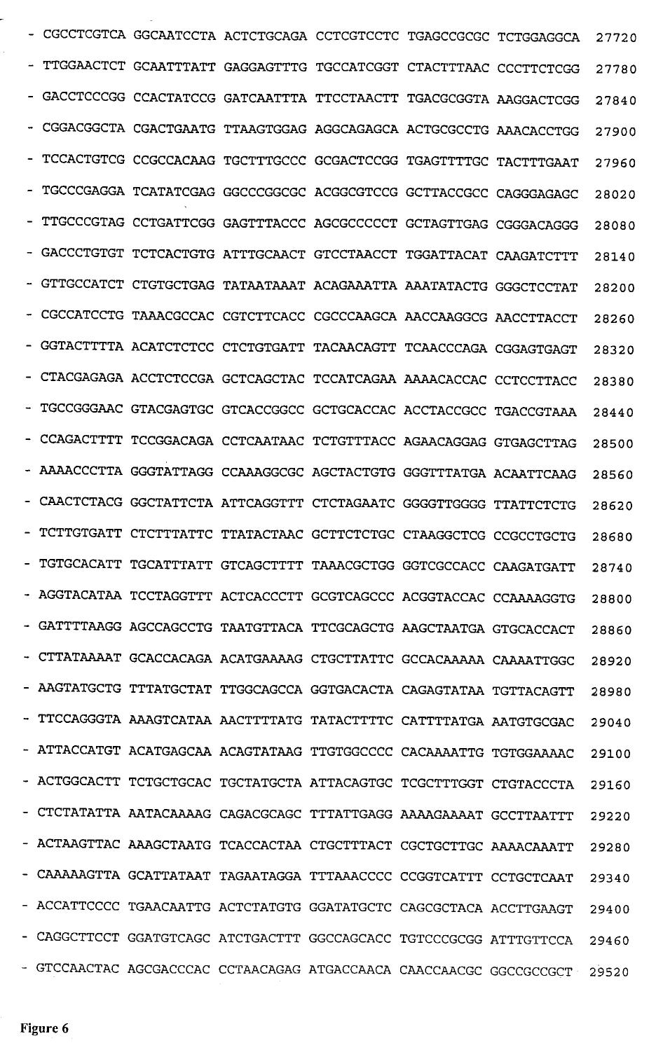

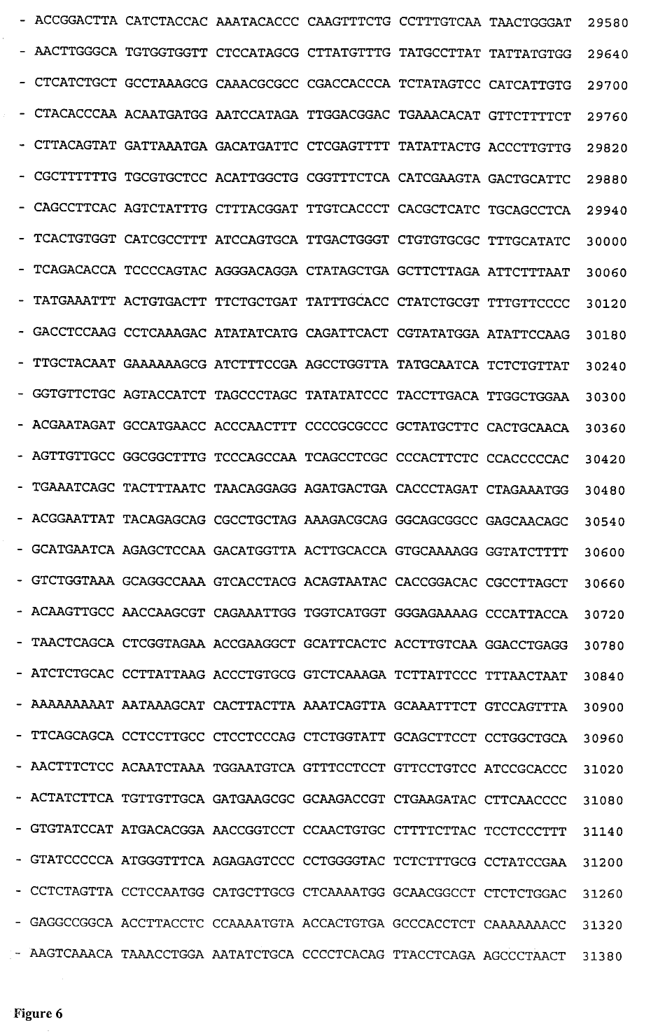

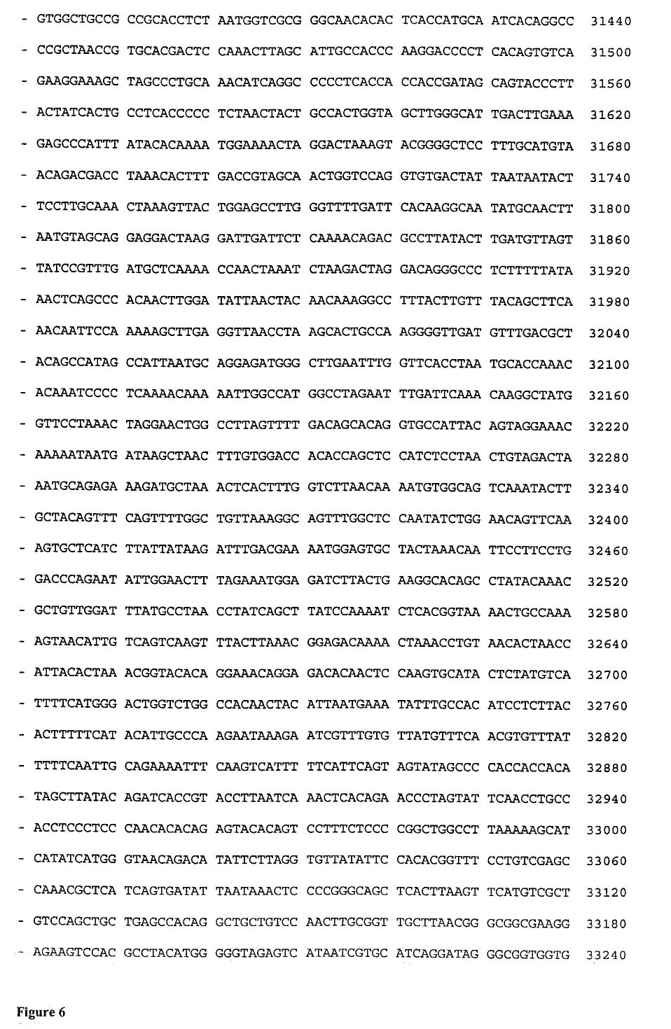

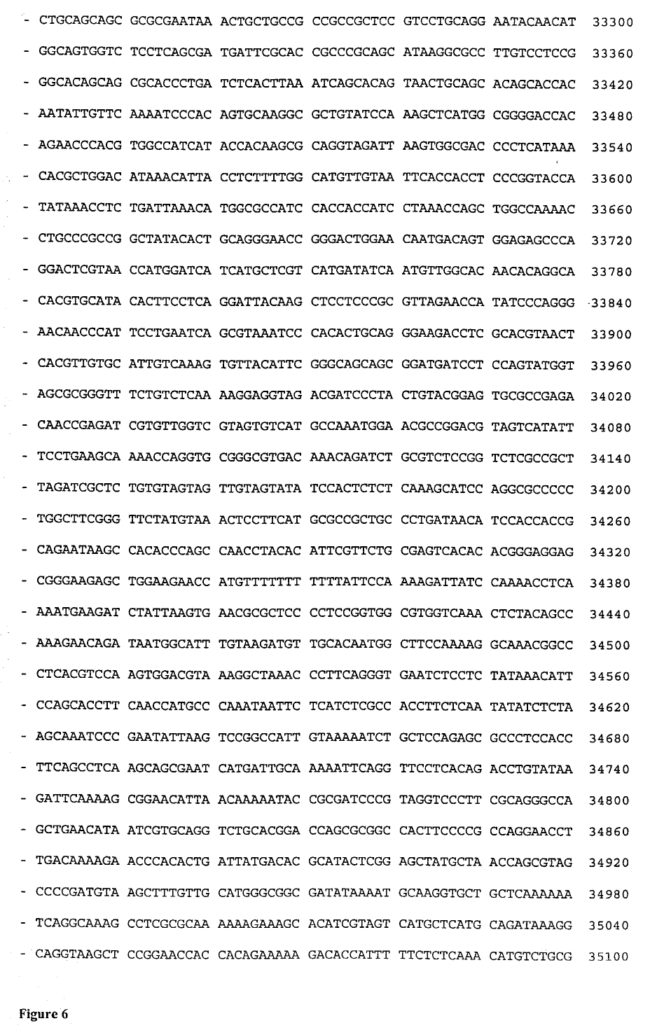

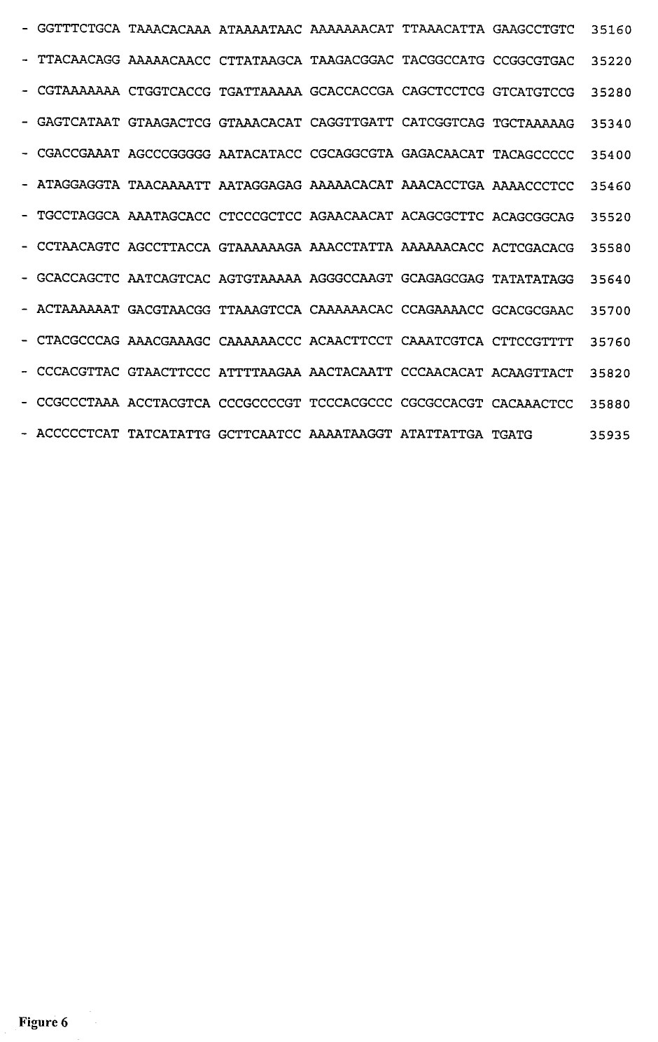

When these extracts were immunoprecipitated with the monoclonal anti-F antibody under mild-salt conditions and submitted to SDS-PAGE, a clear band of non-tagged wt pIX protein was revealed with the anti-pIX antibody as indicated in FIG. 5, in addition to the F-tagged protein variants. As a control, the non-tagged pIX was expressed in the absence of F-tagged pIX and could not be detected in the anti-F immunoprecipitate (not shown). The results strongly suggest therefore that the non-tagged wt pIX was co-precipitated together with the F epitope-tagged wt pIX (F/IXwt, FIG. 5, lane 1). That this co-precipitation involved the leucine-repeat domain, was demonstrated by the observation that single point-mutations (E113K, Q106K, V117D) or a double point mutation (L-V) disrupting this structure reduced or abolished the carrying effect (FIG. 5, compare lane 1 with lanes 5-8). Similarly, a mutant lacking the C-terminal mid-part of the leucine-repeat (delta 111-140) did not co-precipitate wt pIX (data not shown). As expected, mutations within either the N-terminal domain (delta 22-23 and delta 26-28, FIG. 5; or delta 13-15 and delta 31-39, not shown) had no effect on the interaction. By contrast, the central alanine-rich element (delta 63-70) of pIX was essential for co-precipitation (FIG. 5, compare lanes 1 and 4) and is therefore also involved in the oligomerisation process. Furthermore, the observation that the same mutations affect both transactivation and interaction properties of pIX suggests that the two activities may be linked.

### Example 5

### pIX Accumulates in Virus-induced Clear Amorphous Inclusions and Induces their Formation by Itself

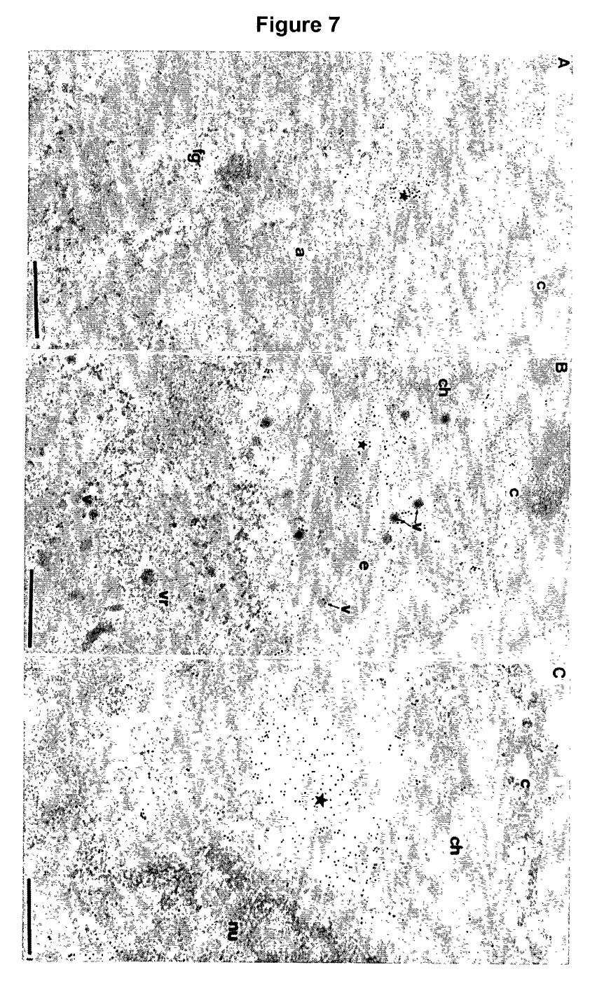

Previously, it has been shown by immunofluorescence staining with pIX-specific antibodies, that pIX was predominantly associated with infected cell nuclei, in accordance with the transcriptional properties of the protein. [Lutz et al., 1997, J. Virol. 71, 5102-5109] In addition, it could be observed that the nuclear staining of pIX was evolutive, showing a speckled distribution at later times of infection. To study the intranuclear distribution of pIX protein more precisely, Ad5-infected A549 cells were examined by immunoelectron microscopy at different times post-infection (pi), from 16 to 30 h pi, in order to observe the accumulation of pIX as a function of the successive steps of nuclear alteration during infection. These experiments demonstrated that protein pIX actively induces specific nuclear clear amorphous (c.a.) inclusions : FIG. 7A represents Ad2-infected A549 cells at intermediate stage of nuclear transformation (14-16 h pi) processed for immunogold labelling with anti-pIX polyclonal antibody on lowicryl sections of formaldehyde-fixed cells. Gold particles are scattered over the fibrillo-granular network (fg), one component of the viral region, and accumulate over an enclosed small irregularly-shaped clear amorphous inclusion (c.a. inclusion; star). The accumulation site of viral single-stranded DNA (a), the other compartment of the viral region, is entirely devoid of pIX protein. c: cytoplasm. Bar, 0.5 μm. As shown in FIG. 7B, later stages of Ad-mediated nuclear transformation (24-30 h pi) are characterised by the presence of progeny viruses. The roughly spherical c.a. inclusion (star) is intensely and homogeneously labelled It is located in the electron-translucent region (e) which separates the perinuclear layer of host chromatin (ch) from the large, centrally-located viral region (vr). Some viruses (v), both scattered in the electron-translucent region and clustered within the viral region are labelled. C : cytoplasm, Bar 0.5 μm. As shown in FIG. 7C, overexpression of recombinant pIX protein induces the accumulation of the protein within newly-formed c.a. inclusions: A549 cells were transfected with the vector expressing the untagged wild type pIX. Gold particles accumulate over the entire surface of an ovoid c.a. inclusion present in the nucleoplasm. Ti clearly appears that the labelled inclusion (star) is similar to those observed in (B) following adenovirus infection.

No significant labelling was observed with the anti-pIX antibody before 16 h pi (not shown). At this time, the infected cells were mainly at the intermediate stage of nuclear alteration. Among several virus-induced structures (including sites of viral DNA replication, transcription and sites of viral genome or single-stranded viral DNA accumulation), pIX was detected in small, irregularly shaped or spherical, clear amorphous inclusions. At a later stage (24-30 h pi), when a central viral compartment and a perinuclear electron-translucent area with protein crystals and isolated viruses were apparent, pIX remained concentrated within the c.a. inclusions which became more frequent and spherical. Sometimes two or three of these inclusions were juxtaposed . In addition, pIX was observed over the crystalline arrays of viruses and the isolated viruses particles .

Hence, since pIX protein is efficiently neosynthesized and belongs to the late phase of infection, the protein is predominantly associated to c.a. inclusions which are dynamic in their shape and location in the nucleus. The amount of pIX in the cell increases as the infection progresses, and the pIX protein accumulates in the nucleus. Irrespective of their shape, size and location within the nucleus, they were always intensely and homogeneously labelled with the anti-pIX antibody.

The ultrastructural data suggest that pIX is the main component of the virus-induced c.a. inclusions. To determine whether pIX was directly responsible for their occurrence, it was attempted to express the protein in cells by transfecting with a wild type pIX expression vector, i.e. in the absence of any other viral protein. The morphology of pIX-expressing cells was similar to that of non-transfected cells except for the additional presence of c.a. inclusions in the nucleoplasm, identical to those observed in lytically infected cells . Depending on the amount of expressed pIX (i.e. as a function of time post-transfection), c.a. inclusions were variable in size and frequency, but always showed the same amorphous aspect (data not shown). Immunogold detection of pIX protein resulted in an intense labelling of each c.a. inclusion and in a slight labelling of the surrounding nucleoplasm and cytoplasm. Therefore, in the absence of other viral proteins, pIX is able to induce the formation of c.a. inclusions similar to those induced by Ad infection.

Specific immunofluorescence staining of cells bearing wt Ad5 or transfected with the wt pIX-expressing vector revealed a speckled distribution of the protein in the nucleus , most likely corresponding to the accumulation of pIX within the c.a. inclusions observed by electron microscopy. To identify peptidic domains of pIx which may be responsible of the formation of c.a. inclusions, the effect of the above-mentioned set of mutations (see FIG. 2A) on the nuclear distribution of pIX in transfected cells was examined. Mutations affecting either the N-terminal (delta 13-15, delta 22-23, delta 26-28, delta 31-39) or central (delta 63-70) domains of pIX did not affect the formation and nuclear location of the c.a. inclusions, since they all yielded the same speckled distribution (data not shown), as the wild type protein upon transfection. By contrast, alteration of the leucine-repeat of pIX by modification of the net charge of specific residues (mutations E1 13K or Q106K) drastically changed the intracellular distribution of the corresponding pIX variants which were then confined to the cytoplasm, as revealed by immunofluorescence staining. Furthermore, although the overall level of mutant expression was similar to that of the wild type protein (as revealed by Western blotting, data not shown), this variant accumulated with a micro-speckled pattern.

Similarly, both point mutations (L114P, V 117D, L-V) and deletions (delta 111-140), affecting the integrity of the leucine-repeat, abolished formation of the c.a. inclusions and resulted in a diffuse distribution of the altered pIX throughout the nucleus and cytoplasm . Fusion of the F epitope-tag, at the C-terminal end of wild type pIX (IX/F), also prevented the nuclear accumulation of the protein and induced the same diffuse pattern, despite the integrity of the leucine-repeat. This effect, most likely related to the steric hindrance imposed by the tag, suggests that free access to the leucine-repeat was essential for nuclear retention of pIX. Interestingly, when cells were cotransfected with vectors expressing the untagged (wt pIX) and C-terminally F-tagged (IX/F) proteins, both types of proteins accumulated within the same inclusion bodies, as revealed by the merged immunofluorescence staining . It appears therefore that the unfocused IX/F protein distribution was redirected by the coexpressed wt pIX into its corresponding nuclear accumulation sites. By contrast, when the F-tagged partner of the cotransfection carried an alteration within the leucine-repeat (IX/F:L-V), this recruitment was nearly abolished, as shown by the persistent diffuse distribution pattern of the F-tagged mutant.

Together, the results presented herein clearly indicate that the leucine-repeat of pIX is chiefly involved in the formation and localisation of the c.a. inclusions. Furthermore, the data strongly suggest that the assembly of the pIX-specific inclusion bodies is an active process driven by pIX itself.

Discussion

Viruses, as obligatory cell parasites, usually evolved towards the highest possible degree of simplification of their structure and components to reach at minimal expense the most efficient rates of proliferation. Adenoviruses comply with this rule, not only in exploiting the coding capacity of their genome by using alternative reading frames, but also in producing proteins with multiple biological activities. The product of the Ad gene IX is an example of such multifunctional proteins: pIX (140 residues) is a structural component of the viral capsid, acts as a transcriptional activator and accumulates in infected cell nuclei as specific structures (c.a. inclusions), the function of which remains to be established. In the present study, an extensive series of site-directed mutagenesis of pIX was performed in order to define the corresponding functional domains of the protein.

Structural involvement of pIX in viral capsid assembly

The pIX protein has previously been described as a capsidic cement between the viral hexons, thereby optimising DNA packaging capacity and thermal stability of Ad virions. Based on immunochemical approaches, it had been suggested that the N-terminal portion of pIX sticks inside the capsid while its C-terminal part points outwards. The results shown herein indicate that residues 22-28 are essential for pIX incorporation into the capsid, virion thermostability and elevated viral production. The importance of these residues is further supported by their high degree of conservation among human and animal Ad serotypes. Additional residues within the N-terminal region of pIX likely contribute to this function, as suggested by the effect of the delta 13-15 deletion which did not impair pIX capsidic integration but affected virion stability.

Interestingly, the present results rule out any contribution of the putative coiled-coil element of pIX to these capsid properties. This clearly indicates that the corresponding capsidic interactions occur through other elements than the coiled-coil domain and suggests that the N-terminal element defined above fulfils this function. Since coiled-coil elements have been shown to be responsible for the trimerisation or oligomerisation of other proteins, like the well documented yeast GCN4 or HIV-1 gp41, it is likely that the coiled-coil element of pIX might also be involved in the trimerisation of this protein as it occurs in the GON complexes. This raises the question of whether the pIX molecules are still organised as trimers in the leucine-repeat mutants or whether the N-terminal domain of pIX, in close contact with the hexon molecules, is primarily responsible for the integration of pIX trimers into the virion capsid. Clearly, additional experiments, including co-immunoprecipitation assays, three-dimensional structure analyses will be required to solve these questions.

Transcriptional activity of pIX

It has previously been reported that recombinant pIX exhibits properties of a transcriptional activator when assayed in transfection experiments or in a reconstituted in vitro transcription system. By the present data, it was shown that the integrity of the leucine-repeat element of pIX is mandatory for this transactivating function, while the highly conserved residues within the N-terminal half of the molecule are not associated to this activity. Larger deletions in the N-terminal region (11-74) or just removing the alanine-rich element (63-70) also impaired pIX transcriptional activity, despite preserving the coiled-coil domain. As suggested by predictive structure analyses (data not shown), the poly-alanine stretch may serve a hinge function in the pIX molecule. It is therefore possible, that mutations to this element disrupt the global structure of pIX and thereby affect its essential functions.

Additional evidence (M.R.-C. et al., in preparation) revealed that, soon after cell entry and virion decapsidation, the released capsidic pIX accumulates in the cell nucleus, as soon as 45 min pi until 6 h pi. These pIX molecules may then activate the highly responsive E1a promoter, thus behaving as a ready-to-use transactivator, much like the VP16 factor in the case of HSV-1 infection. Furthermore, it is likely that the newly synthesised pIX, which starts accumulating at intermediate times pi, also contributes to the activation of the late phase of Ad infection by stimulating MLP activity. Thus, it will be of interest to decipher the molecular mechanism of pIX-mediated transactivation.

As expected from the absence of basic residues flanking its leucine-repeat (to make up a bona fide basic-leucine-zipper) and from the lack of any other DNA recognising motif, pIX has no DNA-binding activity (data not shown). By contrast, as revealed by co-immunoprecipitation experiments, the leucine-repeat is involved in the interaction of pIX with itself, allowing its homo-dimerisation or -oligomerisation via a coiled-coil structure. The fact that the same mutations affect both pIX self-interaction and transactivation properties suggests that the two activities are directly correlated. Alternatively, or in addition, pIx might interact through this leucine-repeat element with components of the transcription apparatus: preliminary results indeed suggest that pIX contacts specific RNA polymerase II subunits and general transcription factors, thus mimicking other viral transactivators like Ad E1a, HSV-1 pX, or VP16.

Interestingly, one feature that is shared by all pIX-responsive promoters assayed so far, whether from viral or cellular origin, is the presence of a canonical TATA box. It will be of interest to identify the mechanism of this specific promoter targeting. It is tempting to speculate that such a promoter preference for pIX is of some advantage for Ad propagation since all viral promoters, with the exception of the E2 promoter, contain a TATA box. This hypothesis is further supported by the fact that only episomal genes (i.e. either in plasmidic or viral form), but not chromosome-integrated reporters, have been found to be responsive to pIX.

Nuclear accumulation structures of pIX

Despite the absence of any detectable homology with consensus nuclear localisation signals (NLS) in its peptide sequence, pIX concentrates within the cell nucleus. hherein, it has been shown that the leucine-repeat of pIX is essential for this nuclear accumulation, as well as for the formation of the c.a. inclusions. Together, these observations indicate that pIX, by virtue of its low molecular weight (M.W.), freely diffuses from the cytoplasm to the nucleus where it is retained, most likely through interactions involving the leucine-repeat and specific nuclear components. Preliminary biochemical evidence suggest that pIX actually associates with fractions of the nuclear matrix . It is therefore likely that the targeting of the nuclear matrix by pIX constitutes the initial step in the assembly of the c.a. inclusions, providing a nucleation point for pIX oligomerisation. Since these nuclear structures are built up in the absence of any other viral protein except pIX, it is to be concluded that they reflect an intrinsic property of pIX.

The leucine-repeat plays a central role in both pIX transcriptional activity and ability to form c.a. inclusions. However several lines of evidence clearly indicate that these functions are two independent properties of pIX: (i) during the late phase of infection, c.a. inclusions were always found excluded from the viral transcription sites; (ii) neither RNA polymerase II nor primary transcripts could be detected within the c.a. inclusions from Ad-infected cells or cells transfected with wild type pIX expressing vectors (data not shown); (iii) a mutant lacking part of its alanine-rich element (delta 63-70) lost its capacity to transactivate the E1a promoter (FIG. 4) and to self-associate under immuno-precipitation conditions (FIG. 5), but retained its ability to accumulate into nuclear c.a. inclusions (data not shown); (iv) conversly, fusion of the F epitope to the C-terminal end of pIX (IX/F), completely abolished the ability of the protein to form c.a. inclusions in the nucleus, but did not affect its transcriptional properties (FIG. 4, lane 15), unless the structural integrity of the leucine-repeat was altered (FIG. 4, lane 16). The most simple interpretation to account for the apparent unrelatedness of these pIX functions, is that pIX might exert its transcriptional properties only at low concentrations (i.e. at initial times of infection and just after the onset of pIX synthesis) while it starts forming nuclear inclusions at higher concentrations by accumulating on nuclear matrix structures, as observed later in infection or after transfection. The finding that addition of the F-tag at the C-terminal end of pIX differentially affects pIX transcriptional activity and c.a. inclusion formation, may therefore merely reflect the preferential impairment of contacts implicated in the assembly of c.a. inclusions, compared to those required for promoter transactivation.

The physiological role of these c.a. inclusions remains puzzling. The possibility that they represent viral protein storage sites, serving as a source of readily mobilisable proteins for subsequent virus assembly, seems unlikely for the following reasons: (i) their formation is induced exclusively by PIX, whose synthesis is disconnected from that of all the other virion proteins, in Ad-infected cells; (ii) although low amounts of hexon can be observed in these structures at late times pi, no penton and fiber proteins can be detected.

Similar globular structures have been described in cells infected with HSV-1 where virus-induced “finely granular translucent patches” were observed late in infection. These structures have been shown to contain viral capsid proteins and, in addition, to concentrate specific cellular proteins like the promyelocytic leukaemia (PML) and SP100 proteins which are diverted from the so-called PML oncogenic domains (PODs) that are the normal PML and SP100 accumulation sites. The detection of PML and SP100 proteins in the c.a. inclusions of Ad-infected cells further stresses the similarity between the two virus-induced structures. This observation also raises the interesting possibility that pIX interferes with the function of PML and related proteins, thus relaying, during later times in infection, the effect of the Ad early E4orf3 product which has also been shown to delocalise PML from the PODs. We are currently exploring these hypotheses.

It appears therefore that pIX plays multiple functions during infection. Interestingly, it shares some of these finctions with the product of the other Ad intermediate gene, pIVa2, both proteins are transcriptional activators, take part in the viral-induced alterations of the host cell by accumulating as specific nuclear inclusions and are present in the mature virion particles. No doubt that these multifaceted viral entities have yet additional secrets to reveal and thus clearly deserve attention when designing Ad-based vectors for gene therapy protocols.

### Example 6

### Insertion of a Polylysine Binding Moiety in the pIX Protein

The human embryonic kidney 293 cell line (ATCC, Rockville, Md., USA) and the CHO cell line (ATCC; CCL-61) were grown at 37° C. in DMEM supplemented with 10% Fetal Calf Serum.

Construction ofplX-modified viral genomes

pIX coding sequence was mutated as described above using the QuickChange site-directed mutagenesis system (Stratagene), to introduce mutations in pIX coding sequence: L114P, V117D and the double mutation L114P-V117D, respectively.

Introduction of 7K and Gly-Ser-(Ser-Ala)₄-Gly-Ser-(K)₇peptides between Leul31 and Lys132 residues within C terminal part ofpIX

The method using the QuickChange site-directed mutagenesis system (Stratagene) allowed to introduce the restriction site BanHI between the base codons encoding Leu131 and Lys132 within the pIX coding sequence. In this case, the following sense and antisense oligonucleotides were used : 5′-cgc cag cag gtt tct gcc ctg gga tcc aag gct tcc tcc cct ccc aat gcg g3′ (SEQ ID NO: 22) and 5′-c cgc att ggg agg gga gga agc ctt gga tcc cag ggc aga aac ctg ctg gcg-3′ (SEQ ID NO:23).

The oligonucleotides 5′-ga tcc aag aag aag aag aag aag aag taa g-3′ (SEQ ID NO:24) and 5′-ga tcc tta ctt ctt ctt ctt ctt ctt ctt tga tcc cgc cga cgc gga ggc gga gcc gct g-3′ (SEQ ID NO: 25) z were hybridized to each other and cloned into the Bam-HI site to directly introduce the 7K peptide within the C-terminal part of pIX. Alternatively, the oligonucleotides 5′-ga tcc agc gcc tcc gcc tcc gcg tcg gcg gga tca aag aag aag aag aag aag aag taa g-3′ (SEQ ID NO: 26) and 5′-ga tcc tta ctt ctt ctt ctt ctt ctt ctt tga tcc cgc cga cgc gga ggc gga gcc gct gga tcc g-3′ (SEQ ID NO:27) were hybridized to each other and cloned into the BamHI site to directly introduce the 7K binding moiety connected to the pIX sequence via the spacer Gly-Ser-(Ser-Ala)₄-Gly-Ser.

Introduction of the 7K peptide between Q127 and Val128 residues within the C-terminal part of pIX

Alternatively, the QuickChange site-directed mutagenesis system (Stratagene) was also used to introduce in two steps the 7K peptide between the residues Q127 and V128 within the C-terminal part of pIX. Following sense and antisense oligonucleotides were used in the first step: 5′-g ctg ttg gat ctg cgc cag cag aag aag aag aga tct gcc ctg aag gct tcc tcc cct ccc aat gcg g-3′ (SEQ ID NO:28) and 5′-c cgc att ggg agg gga gga agc ctt cag ggc aga tct ctt ctt ctt ctg ctg geg cag atc caa cag c-3′ (SEQ ID NO: 29), and then in the second step, the following oligonucleotides : 5′-g ctg ttg gat ctg cgc cag cag aag aag aag aag aaa aaa aaa taa tct gcc ctg aag gct tcc tcc cct ccc aat gcg g-3′ (SEQ ID NO: 30) and 5′-c cgc att ggg agg gga gga agc ctt cag ggc aga tta ttt ttt ttt ctt ctt ctt ctt ctg ctg gcg cag atc caa cag c-3′ (SEQ ID NO: 31).

The amino acid sequence of the modified pIX protein can be read as follows starting from L in position 100 (see SEQ ID NO:32 and 33, respectively):

LTALLAQLDSLTREP(114)NVD(117)SQQLLDLRQQKKKKKKK (7K binding moiety)
or
LTALLAQLDSLTREP(114)NVD(117)SQQLLDLRQQVSALKGS(SA)₄SKKKKKKK(spacer / 7K mo-
iety).

All vectors contain, in addition to alterations of gene pIX, a deletion in E1 and in E3 and comprise in replacement of E1 the LacZ gene (encoding beta galactosidase) driven by the CMV promoter.

The infection efficiency of the 7K-containing pIX constructs for a number of tumoral cells was evaluated in comparison to the conventional targeted adenovirus comprising the 7K binding moiety comprised in the capsid fiber (Ad Fb 7K described by Leissner et al (2001, Gene therapy 8, 49-57).

Cell lines infected by 7K modified Ad

The human adenocarcinoma prostatic PC3 (ATCC CRL-1435) and LN-CAP (ATCC CRL-10955) cell lines, the human squamous carcinoa from head and neck SqCCYl (Dr. Reuben Lotan Univ Texas, Houston, USA) cell line, and the human breast cancer T47D (ATCC HTB-133), H3396 (BMS) and SKBR3 cell lines were grown at 37° C. in DMEM or RPMI supplemented with 10% Fetal Calf Serum and antibiotics.

Conditions of infection

Viruses in the range of concentrations 10, 100 and 1000 particles/cell were added to target cell monolayers for 1 hour at 4° C. The inoculum was removed and the cells were washed twice with cold medium before they were incubated for 48 h at 37° C. Cells were then fixed and stained for determining β-galactosidase activity, as previously described by P. Leissner et al. (2001, Gene Therapy 8, 49-57). Infected cells were then counted. Alternatively, the β-galactosidase activity of whole cell lysate was monitored using a chemiluminescent substrate (luminescent β-galactosidase detection kit, Clontech, Palo Alto), as also described by P. Leissner et al. (2001, Gene Therapy 8, 49-57).

For all tumor cell lines tested including human SKBR3, T47D, H3326, A549 cells and murine Renca, B16F0, the infectivity of Ad-pIX 7K was superior by 10 to 100 fold compared to the other vectors and especially the conventional targeting vector Ad-Fb-7K comprising the 7K binding moiety inserted at the C-terminus of the fiber.

In these studies, it was demonstrated that peptide-binding moieties can be inserted within the C-terminal part of the Ad pIX protein. The superior infection of Ad-pIX 7K into a variety of human and murine tumor cells is consistent with the fact that many tumor cells have an increased expression of heparan sulfate proteoglycans on their surface. Therefore, retargeting the vector with tumor-specific ligands using the pIX protein will expand the applicability of adenoviral vectors for cancer gene therapy.

Bibliography

1. Ace, C. I., T. A. McKee, J. M. Ryan, J. M. Cameron, and C. M. Preston. 1989. Construction and characterization of a herpes simplex virus type 1 mutant unable to transinduce immediate-early gene expression. Journal of Virology 63:2260-9.

2. Akalu, A., H. Liebermann, U. Bauer, H. Granzow, and W. Seidel. 1999. The subgenus-specific C-terminal region of protein IX is located on the surface of the adenovirus capsid. J Virol 73:6182-7.

3. Alestrom, P., G. Akusjarvi, M. Perricaudet, M. B. Mathews, D. F. Klessig, and U. Pettersson. 1980. The gene for polypeptide IX of adenovirus type 2 and its unspliced messenger RNA. Cell 19:671-81.

4. Ali, S., Y. Lutz, J. P. Bellocq, M. P. Chenard-Neu, N. Rouyer, and D. Metzger. 1993. Production and characterization of monoclonal antibodies recognising defined regions of the human oestrogen receptor. Hybridoma 12:391-405.

5. Bellon, G., L. Michel-Calemard, D. Thouvenot, V. Jagneaux, F. Poitevin, C. Malcus, N. Accart, M. P. Layani, M. Aymard, H. Bernon, J. Bienvenu, M. Courtney, G. Doring, B. Gilly, R. Gilly, D. Lamy, H. Levrey, Y. Morel, C. Paulin, F. Perraud, L. Rodillon, C. Sene, S. So, F. Touraine-Moulin, A. Pavirani, and et al. 1997. Aerosol administration of a recombinant adenovirus expressing CFTR to cystic fibrosis patients: a phase I clinical trial. Hum Gene Ther 8:15-25.

6. Bocco, J. L., B. Reimund, B. Chatton, and C. Kedinger. 1993. Rb may act as a transcriptional co-activator in undifferentiated F9 cells [published erratum appears in Oncogene 1994 Mar;9(3):999]. Oncogene 8:2977-86.

7. Boulanger, P., P. Lemay, G. E. Blair, and W. C. Russell. 1979. Characterization of adenovirus protein IX. Journal of General Virology 44:783-800.

8. Boulikas, T. 1994. Putative nuclear localization signals (NLS) in protein transcription factors. Journal of Cellular Biochemistry 55:32-58.

9. Burnett, R. M. 1985. The structure of the adenovirus capsid. II. The packing symmetry of hexon and its implications for viral architecture. Journal of Molecular Biology 185:125-43.

10. Burnett, R. M., M. G. Grutter, and J. L. White. 1985. The structure of the adenovirus capsid. I. An envelope model of hexon at 6 A resolution. Journal of Molecular Biology 185:105-23.

11. Carvalho, T., J. S. Seeler, K. Ohman, P. Jordan, U. Pettersson, G. Akusjarvi, M. Carmo-Fonseca, and A. Dejean. 1995. Targeting of adenovirus E1A and E4-ORF3 proteins to nuclear matrix-associated PML bodies. J Cell Biol 131:45-56.

12. Chartier, C., E. Degryse, M. Gantzer, A. Dieterle, A. Pavirani, and M. Mehtali. 1996. Efficient generation of recombinant adenovirus vectors by homologous recombination in Escherichia coli. J Virol 70:4805-10.

13. Chen, C., and H. Okayama. 1987. High-efficiency transformation of mammalian cells by plasmid DNA. Mol Cell Biol 7:2745-52.

14. Christ, M., B. Louis, F. Stoeckel, A. Dieterle, L. Grave, D. Dreyer, J. Kintz, D. Ali Hadji, M. Lusky, and M. Mehtali. 2000. Modulation of the inflammatory properties and hepatotoxicity of recombinant adenovirus vectors by the viral E4 gene products. Hum Gene Ther 11:415-27.

15. Chroboczek, J., F. Bieber, and B. Jacrot. 1992. The sequence of the genome of adenovirus type 5 and its comparison with the genome of adenovirus type 2. Virology 186:280-5.

16. Clayman, G. L., A. K. el-Naggar, S. M. Lippman, Y. C. Henderson, M. Frederick, J. A. Merritt, L. A. Zumstein, T. M. Timmons, T. J. Liu, L. Ginsberg, J. A. Roth, W. K. Hong, P. Bruso, and H. Goepfert. 1998. Adenovirus-mediated p53 gene transfer in patients with advanced recurrent head and neck squamous cell carcinoma. Journal of Clinical Oncology 16:2221-32.

17. Colby, W. W., and T. Shenk. 1981. Adenovirus type 5 virions can be assembled in vivo in the absence of detectable polypeptide IX. J Virol 39:977-80.

18. Connelly, S., T. A. Smith, G. Dhir, J. M. Gardner, M. G. Mehaffey, K. S. Zaret, A. McClelland, and M. Kaleko. 1995. In vivo gene delivery and expression of physiological levels of functional human factor VIII in mice. Hum Gene Ther 6:185-93.

19. Crystal, R. G., E. Hirschowitz, M. Lieberman, J. Daly, E. Kazam, C. Henschke, D. Yankelevitz, N. Kemeny, R. Silverstein, A. Ohwada, T. Russi, A. Mastrangeli, A. Sanders, J. Cooke, and B. G. Harvey. 1997. Phase I study of direct administration of a replication deficient adenovirus vector containing the E. coli cytosine deaminase gene to metastatic colon carcinoma of the liver in association with the oral administration of the pro-drug 5-fluorocytosine. Hum Gene Ther 8:985-1001.

20. Davis, A. R., N. A. Wivel, J. L. Palladino, L. Tao, and J. M. Wilson. 2000. Construction of adenoviral vectors. Methods Mol Biol 135:515-23.

21. Fallaux, F. J., A. Bout, I. van der Velde, D. J. van den Wollenberg, K. M. Hehir, J. Keegan, C. Auger, S. J. Cramer, H. van Ormondt, A. J. van der Eb, D. Valerio, and R. C. Hoeben. 1998. New helper cells and matched early region 1-deleted adenovirus vectors prevent generation of replication-competent adenoviruses. Hum Gene Ther 9:1909-17.

22. Furcinitti, P. S., J. van Oostrum, and R. M. Burnett. 1989. Adenovirus polypeptide IX revealed as capsid cement by difference images from electron microscopy and crystallography. Embo J 8:3563-70.

23. Gahery-Segard, H., V. Molinier-Frenkel, C. Le Boulaire, P. Saulnier, P. Opolon, R. Lengagne, E. Gautier, A. Le Cesne, L. Zitvogel, A. Venet, C. Schatz, M. Courtney, T. Le Chevalier, T. Tursz, J. G. Guillet, and F. Farace. 1997. Phase I trial of recombinant adenovirus gene transfer in lung cancer. Longitudinal study of the immune responses to transgene and viral products. Journal of Clinical Investigation 100:2218-26.

24. Ghosh-Choudhury, G., Y. Haj-Ahmad, and F. L. Graham. 1987. Protein IX, a minor component of the human adenovirus capsid, is essential for the packaging of full length genomes. EMBO Journal 6:1733-9.

25. Gonzalez, L., Jr., D. N. Woolfson, and T. Alber. 1996. Buried polar residues and structural specificity in the GCN4 leucine zipper. Nature Structural Biology 3:1011-8.

26. Graham, F. L., and L. Prevec. 1992. Adenovirus-based expression vectors and recombinant vaccines. Biotechnology 20:363-90.

27. Graham, F. L., J. Smiley, W. C. Russell, and R. Nairn. 1977. Characteristics of a human cell line transformed by DNA from human adenovirus type 5. Journal of General Virology 36:59-74.

28. Green, S., and P. Chambon. 1988. Nuclear receptors enhance our understanding of transcription regulation. Trends Genet 4:309-14.

29. Harbury, P. B., P. S. Kim, and T. Alber. 1994. Crystal structure of an isoleucine-zipper trimer. Nature 371:80-3.

30. Harbury, P. B., T. Zhang, P. S. Kim, and T. Alber. 1993. A switch between two-, three-, and four-stranded coiled coils in GCN4 leucine zipper mutants. Science 262:1401-7.

31. Hayashi, F., R. Ishima, D. Liu, K. I. Tong, S. Kim, D. Reinberg, S. Bagby, and M. Ikura. 1998. Human general transcription factor TFIIB: conformational variability and interaction with VP16 activation domain. Biochemistry 37:7941-51.

32. Horikoshi, N., K. Maguire, A. Kralli, E. Maldonado, D. Reinberg, and R. Weinmann. 1991. Direct interaction between adenovirus E1A protein and the TATA box binding transcription factor IID. Proceedings of the National Academy of Sciences of the United States of America 88:5124-8.

33. Hu, J. C., N. E. Newell, B. Tidor, and R. T. Sauer. 1993. Probing the roles of residues at the e and g positions of the GCN4 leucine zipper by combinatorial mutagenesis. Protein Sci 2:1072-84.

34. Hurst, H. C. 1995. Transcription factors 1: bZIP proteins. Protein Profile 2:101-68.

35. Hurst, H. C. 1994. Transcription factors. 1: bZIP proteins. Protein Profile 1:123-68.

36. Jeanmougin, F., J. D. Thompson, M. Gouy, D. G. Higgins, and T. J. Gibson. 1998. Multiple sequence alignment with Clustal X. Trends Biochem Sci 23:403-5. 37. Landschulz, W. H., P. F. Johnson, and S. L. McKnight. 1988. The leucine zipper: a hypothetical structure common to a new class of DNA binding proteins. Science 240:1759-64.

38. Legrand, V., D. Spehner, Y. Schlesinger, N. Settelen, A. Pavirani, and M.

Mehtali. 1999. Fiberless recombinant adenoviruses: virus maturation and infectivity in the absence of fiber. Journal of Virology 73:907-19.

39. Lupas, A. 1996. Coiled coils: new structures and new functions. Trends in Biochemical Sciences 21:375-82.

40. Lupas, A. 1996. Prediction and analysis of coiled-coil structures. Methods in Enzymology 266:513-25.

41. Lusky, M., L. Grave, A. Dieterle, D. Dreyer, M. Christ, C. Ziller, P. Furstenberger, J. Kintz, D. A. Hadji, A. Pavirani, and M. Mehtali. 1999. Regulation of adenovirus-mediated transgene expression by the viral E4 gene products: requirement for E4 ORF3. J Virol 73:8308-19.

42. Lutz, P., and C. Kedinger. 1996. Properties of the adenovirus IVa2 gene product, an effector of late-phase-dependent activation of the major late promoter. Journal of Virology 70:1396-405.

43. Lutz, P., F. Puvion-Dutilleul, Y. Lutz, and C. Kedinger. 1996. Nucleoplasmic and nucleolar distribution of the adenovirus IVa2 gene product. Journal of Virology 70:3449-60.

44. Lutz, P., M. Rosa-Calatrava, and C. Kedinger. 1997. The product of the adenovirus intermediate gene IX is a transcriptional activator. Journal of Virology 71:5102-9.

45. Puvion-Dutilleul, F., J. P. Bachellerie, N. Visa, and E. Puvion. 1994. Rearrangements of intranuclear structures involved in RNA processing in response to adenovirus infection. Journal of Cell Science 107:1457-68.

46. Puvion-Dutilleul, F., M. K. Chelbi-Alix, M. Koken, F. Quignon, E. Puvion, and H. de The. 1995. Adenovirus infection induces rearrangements in the intranuclear distribution of the nuclear body-associated PML protein. Experimental Cell Research 218:9-16.

47. Puvion-Dutilleul, F., and M. E. Christensen. 1993. Alterations of fibrillarin distribution and nucleolar ultrastructure induced by adenovirus infection. European Journal of Cell Biology 61:168-76.

48. Puvion-Dutilleul, F., V. Legrand, M. Mehtali, M. K. Chelbi-Alix, H. de The, and E. Puvion. 1999. Deletion of the fiber gene induces the storage of hexon and penton base proteins in PML/Sp 100-containing inclusions during adenovirus infection. Biology of the Cell 91:617-28.

49. Puvion-Dutilleul, F., L. Venturini, M. C. Guillemin, H. de The, and E. Puvion. 1995. Sequestration of PML and Sp100 proteins in an intranuclear viral structure during herpes simplex virus type 1 infection. Experimental Cell Research 221:448-61.

50. Qadri, I., H. F. Maguire, and A. Siddiqui. 1995. Hepatitis B virus transactivator protein X interacts with the TATA-binding protein. Proceedings of the National Academy of Sciences of the United States of America 92:1003-7.

51. Rosa-Calatrava, M., F. Puvion-Dutilleul, B. Chatton, and C. Kedinger. 2000. Multifunctional pIX protein plays an active role in Ad-induced alteration of nuclear ultrastructures by targeting PML and SP100.

52. Seeler, J. S., and A. Dejean. 1999. The PML nuclear bodies: actors or extras? Curr Opin Genet Dev 9:362-7.

53. Shu, W., H. Ji, and M. Lu. 1999. Trimerization specificity in HIV-1 gp41: analysis with a GCN4 leucine zipper model. Biochemistry 38:5378-85.

54. Smith, B. T. 1977. Cell line A549: a model system for the study of alveolar type II cell function. American Review of Respiratory Disease 115:285-93.

55. Stewart, P. L., R. M. Burnett, M. Cyrklaff, and S. D. Fuller. 1991. Image reconstruction reveals the complex molecular organization of adenovirus. Cell 67:145-54.

56. Trapnell, B. C., and M. Gorziglia. 1994. Gene therapy using adenoviral vectors. Curr Opin Biotechnol 5:617-25.

57. van Oostrum, J., and R. M. Burnett. 1985. Molecular composition of the adenovirus type 2 virion. Journal of Virology 56:439-48.

58. Xiao, J. H., I. Davidson, H. Matthes, J. M. Gamier, and P. Chambon. 1991. Cloning, expression, and transcriptional properties of the human enhancer factor TEF-1. Cell 65:551-68.

59. Zhang, W., and M. J. Imperiale. 2000. Interaction of the adenovirus IVa2 protein with viral packaging sequences. Journal of Virology 74:2687-93.

1. An adenovirus pIX protein modified by mutation of one or more amino acids of said pIX protein as compared to the corresponding wild-type pIX protein and/or so as to comprise a binding moiety, wherein the presence of said modified pIX protein in a virus or virus-like particle results in an improved gene delivery efficiency in a target cell of said virus or virus-like particle as compared to the gene delivery efficiency of a corresponding virus or virus-like particle containing said corresponding wild-type pIX protein.

2. The adenovirus pIX protein of claim 1, wherein said mutation results in the presence of a binding moiety in the pIX protein.

3. The adenovirus pIX protein of claim 1 or 2, wherein said binding moiety is capable to bind to a target cell.

4. The adenovirus pIX protein of any one of claims 1 to 3, wherein said amino acids to be mutated are selected in the N-terminal part of the protein.

5. The adenovirus pIX protein of any one of claims 1 to 3, wherein said amino acids to be mutated are selected in the C-terminal part of the protein.

6. The adenovirus pIX protein of claim 5, wherein said amino acids to be mutated are selected in the C-terminal leucine-repeat of the protein.

7. The adenovirus pIX protein of claim 6, which shows a mutation selected from the group consisting of
(a) a substitution of the leucine residue at a position corresponding to position 114 of SEQ ID NO:1 by a proline residue;
(b) a substitutions of the valine residue at a position corresponding to position 117
(c) substitution of the leucine residue and the valine residue at positions corresponding to position 114 and 117, respectively, of SEQ ID NO:1 by a proline and an aspartic acid residue, respectively.

8. The adenovirus pIX protein of any one of claims 1 to 7 having a binding moiety which is polylysine.

9. The adenovirus pIX protein of claim 8 comprising the amino acid sequence of SEQ ID NO:32 or 33.

10. A nucleic acid molecule comprising a nucleotide sequence encoding the adenovirus pIX protein of any one of claims 1 to 9.

11. An adenoviral vector comprising the nucleic acid molecule of claim 10.

12. A method for producing a virus or virus-like particle comprising the steps of
a. transforming a suitable host cell with an adenoviral vector according to claim 11;
b. culturing the transformed cell line under conditions suitable to allow formation of a virus or virus-like particle from said adenoviral vector; and
c. recovering the virus or virus-like particle formed in step (b) from the culture.

13. A method for producing a virus or virus-like particle comprising the steps of
a. transforming a suitable host cell with an adenoviral vector which encodes a wild-type pIX protein;
b. modifying the coding sequence for the pIX protein in the adenoviral vector so that the encoded pIX protein is one according to any one of claims 1 to 9;
c. culturing the host cell under conditions suitable to allow formation of a virus or virus-like particle from the adenoviral vector of step (b); and
d. recovering the virus or virus-like particle formed in step (c) from the culture.

14. A virus or virus-like particle comprising the adenovirus pIX protein of any one of claims 1 to 9, the nucleic acid molecule of claim 10 or the adenoviral vector of claim 11 or obtainable by the method of claim 12 or 13.

15. The virus or virus-like particle of claim 14 which is substantially incapable of binding its host cell.

16. A eukaryotic host cell comprising the adenovirus pIX protein of any one of claims 1 to 9, the nucleic acid molecule of claim 10 or the adenoviral vector of claim 11 or being infected with or comprising the virus or virus-like particle of claim 14 or 15.

17. A complementation cell line suitable for producing the virus or virus-like particle of claim 14 or 15 or for applying the method of claim 12 or 13.

18. A pharmaceutical composition comprising the adenoviral vector of claim 11, the virus or virus-like particle of claim 14 or 15, the host cell of claim 16 or the complementation cell line of claim 17, wherein said adenoviral vector, virus or virus-like particle host cell or complementation cell line is capable of expressing a therapeutically useful gene, and optionally a pharmaceutically acceptable carrier.

19. Use of the adenoviral vector of claim 11, the virus or virus-like particle of claim 14 or 15, the host cell of claim 16 or the complementation cell line of claim 17, wherein said adenoviral vector, virus or virus-like particle, host cell or complementation cell line is capable of expressing a therapeutically useful gene, for the preparation of a pharmaceutical composition for gene therapy in a patient in need for such therapy.


SEQUENCE LISTING (33 sequences)

SEQ ID NO: 1 - protein, 140 aa, Adenovirus type 5
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAV

SEQ ID NO: 2 - DNA, 37 bp, Artificial Sequence
  mutagenesis oligonucleotide
gacaacgcgcatgcccccaggggtgcgtcagaatgtg

SEQ ID NO: 3 - DNA, 37 bp, Artificial Sequence
  mutagenesis oligonucleotide
cacattctgacgcacccctgggggcatgcgcgttgtc

SEQ ID NO: 4 - DNA, 46 bp, Artificial Sequence
  mutagenesis oligonucleotide
gatggaagcattgtgagcacaacgcgcatgcccccatgggccgggg

SEQ ID NO: 5 - DNA, 46 bp, Artificial Sequence
  mutagenesis oligonucleotide
ccccggcccatgggggcatgcgcgttgtgctcacaatgcttccatc

SEQ ID NO: 6 - DNA, 39 bp, Artificial Sequence
  mutagenesis oligonucleotide
cccccatgggccggggtgctcgacgtgatgggctccagc

SEQ ID NO: 7 - DNA, 39 bp, Artificial Sequence
  mutagenesis oligonucleotide
cgacctcgggtagtggtcgagcaccccggcccatggggg

SEQ ID NO: 8 - DNA, 46 bp, Artificial Sequence
  mutagenesis oligonucleotide
gggccggggtgcgtcagaatgtgatgcatatgcccgtcctgcccgc

SEQ ID NO: 9 - DNA, 46 bp, Artificial Sequence
  mutagenesis oligonucleotide
gcgggcaggacgggcatatgcatcacattctgacgcaccccggccc

SEQ ID NO: 10 - DNA, 38 bp, Artificial Sequence
  mutagenesis oligonucleotide
ggattctttgacccgggagcccaatgtcgtttctcagc

SEQ ID NO: 11 - DNA, 37 bp, Artificial Sequence
  mutagenesis oligonucleotide
gctgagaaacgacattgggctccgggtcaaagaatcc

SEQ ID NO: 12 - DNA, 33 bp, Artificial Sequence
  mutagenesis oligonucleotide
gggaacttaatgtcgactctcagcagctgttgg

SEQ ID NO: 13 - DNA, 33 bp, Artificial Sequence
  mutagenesis oligonucleotide
ccaacagctgctgagagtcgacattaagttccc

SEQ ID NO: 14 - DNA, 47 bp, Artificial Sequence
  mutagenesis oligonucleotide
ggattctttgacccgggagcccaatgtcgactctcagcagctgttgg

SEQ ID NO: 15 - DNA, 46 bp, Artificial Sequence
  mutagenesis oligonucleotide
ccaacagctgctgagagtcgacattgggctccgggtcaaagaatcc

SEQ ID NO: 16 - DNA, 32 bp, Artificial Sequence
  mutagenesis oligonucleotide
cgccgttggagactccatggaccgcccgcggg

SEQ ID NO: 17 - DNA, 32 bp, Artificial Sequence
  mutagenesis oligonucleotide
cccgcgggcggtccatggagtctccaacggcg

SEQ ID NO: 18 - DNA, 38 bp, Artificial Sequence
  mutagenesis oligonucleotide
ggattctttgacccggaaacttaatgtcgtttctcagc

SEQ ID NO: 19 - DNA, 38 bp, Artificial Sequence
  mutagenesis oligonucleotide
gctgagaaacgacattaagtttccgggtcaaagaatcc

SEQ ID NO: 20 - DNA, 38 bp, Artificial Sequence
  mutagenesis oligonucleotide
gacggctcttttggcaaagcttgattctttgacccggg

SEQ ID NO: 21 - DNA, 38 bp, Artificial Sequence
  mutagenesis oligonucleotide
cccgggtcaaagaatcttgctttgccaaaagagccgtc

SEQ ID NO: 22 - DNA, 52 bp, Artificial Sequence
  mutagenesis oligonucleotide
cgccagcaggtttctgccctgggatccaaggcttcctcccctcccaatgcgg

SEQ ID NO: 23 - DNA, 52 bp, Artificial Sequence
  mutagenesis oligonucleotide
ccgcattgggaggggaggaagccttggatcccagggcagaaacctgctggcg

SEQ ID NO: 24 - DNA, 30 bp, Artificial Sequence
  mutagenesis oligonucleotide
gatccaagaagaagaagaagaagaagtaag

SEQ ID NO: 25 - DNA, 60 bp, Artificial Sequence
  mutagenesis oligonucleotide
gatccttacttcttcttcttcttcttctttgatcccgccgacgcggaggcggagccgctg

SEQ ID NO: 26 - DNA, 60 bp, Artificial Sequence
  mutagenesis oligonucleotide
gatccagcgcctccgcctccgcgtcggcgggatcaaagaagaagaagaagaagaagtaag

SEQ ID NO: 27 - DNA, 66 bp, Artificial Sequence
  mutagenesis oligonucleotide
gatccttacttcttcttcttcttcttctttgatcccgccgacgcggaggcggagccgctggatccg

SEQ ID NO: 28 - DNA, 68 bp, Artificial Sequence
  mutagenesis oligonucleotide
gctgttggatctgcgccagcagaagaagaagagatctgccctgaaggcttcctcccctcccaatgcgg

SEQ ID NO: 29 - DNA, 68 bp, Artificial Sequence
  mutagenesis oligonucleotide
ccgcattgggaggggaggaagccttcagggcagatctcttcttcttctgctggcgcagatccaacagc

SEQ ID NO: 30 - DNA, 80 bp, Artificial Sequence
  mutagenesis oligonucleotide
gctgttggatctgcgccagcagaagaagaagaagaaaaaaaaataatctgccctgaaggcttcctcccctcccaatgcgg

SEQ ID NO: 31 - DNA, 80 bp, Artificial Sequence
  mutagenesis oligonucleotide
ccgcattgggaggggaggaagccttcagggcagattatttttttttcttcttcttcttctgctggcgcagatccaacagc

SEQ ID NO: 32 - protein, 134 aa, Artificial Sequence
  mutant pIX connected with a 7K binding moiety
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTREPNVDSQQLLDLRQQKKKKKKK

SEQ ID NO: 33 - protein, 150 aa, Artificial Sequence
  mutant of Ad5 pIX connected to the 7K binding moiety through the use of a spacer
MSTNSFDGSIVSSYLTTRMPPWAGVRQNVMGSSIDGRPVLPANSTTLTYETVSGTPLETAASAAASAAAATATGIVTDFAFLSPLASSAASRSSARDDKLTALLAQLDSLTREPNVDSQQLLDLRQQVSALKGSSASASASASKKKKKKK